(19) United States
(12) Patent Application Publication
(10) Pub. No.: US 2006/0173022 A1
(43) Pub. Date: Aug. 3, 2006

(54) Heterocyclic amides, method for the production thereof, substances containing said heterocyclic amides, and use thereof as pesticides

(75) Inventor: Wolfgang Schaper, Diedorf (DE)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: Bayer CropScience GmbH, Frankfurt (DE)

(21) Appl. No.: 10/547,004

(22) PCT Filed: Feb. 19, 2004
(86) PCT No.: PCT/EP04/01577

§ 371 Date: Aug. 25, 2005

(30) Foreign Application Priority Data
Feb. 25, 2003 (DE) 103 07 845.2

(51) Int. Cl. A61K 31/506; A61K 31/455; C07D 239/42; C07D 213/46

(52) U.S. Cl. 514/256; 514/355; 544/334; 546/315

(57) ABSTRACT

Amides of the formula (I) and salts thereof 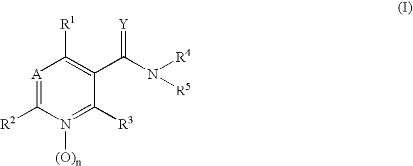
wherein: A is CH or N; Y is O or S; n is 0 or 1; R¹ is (C₁-C₄)-haloalkyl; R² and R³ are identical or different and are hydrogen, (C₁-C₄)-alkyl, (C₁-C₄)-haloalkyl or halogen; R⁴ is hydrogen, (C₁-C₁₀)-alkyl, (C₃-C₁₀)-cycloalkyl, (C₃-C₁₀)-alkenyl or (C₃-C₁₀)-alkynyl, where in the alkyl, cycloalkyl, alkenyl or alkynyl groups mentioned up to three hydrogen atoms may be replaced by halogen, preferably fluorine or chlorine, in the case of fluorine also up to the maximum number; R⁵ is G¹SR⁶, G¹S(O)R⁷, G¹S(O)₂R⁸, G¹OR⁹, G¹NR¹⁰R¹¹, G²CR¹²═NOR¹³, G¹ON═CR¹⁴R¹⁵, G²CR¹²═N—NR¹⁶R¹⁷, G¹NR¹³N═CR¹⁴R¹⁵, G¹NR¹⁸NR¹⁹R²⁰, G¹ONR²¹R²², G¹NR²³OR²⁴, G²CR¹⁰═N⁽⁺⁾(O⁽⁻⁾)R¹², R²⁵ or G¹R²⁶; G¹ is a straight-chain or branched (C₂-C₆)-alkylene moiety, the distance between the amide nitrogen and the radical R⁵ being at least C₂; G² is a straight-chain or branched (C₁-C₆)-alkylene moiety and R⁶ to R²⁶ are as defined in the description; are suitable for controlling pests.

The invention relates to heterocyclic amides, to processes for their preparation, to compositions comprising them and to their use for controlling animal pests, and in particular arthropods, such as insects and arachnids, and helminths.

Owing to the enormous damage caused by insects, for example by feeding on useful plants, stored food, wood and textiles, or else by transferring diseases to man, domestic animals and useful plants, the use of insectides or repellents remains indispensable. Insecticides are an important component of integrated pest control, and their contribution is decisive with respect to harvest yields and yield continuity all over the world.

EP-A 0 580 374, EP-A 1 256 569, JP-A 07025853, JP-A 07010841, JP-A 2003/113179 and JP-A 10101648 disclose N-alkyl-trifluoromethylnicotinamides as pesticides; WOA 01/46152 describes N-alkyl-trifluoromethylnicotinamides as herbicides.

However, since the ecological and economic demands made on modern insecticides are increasing continually, for example with respect to toxicity, selectivity, application rates, formation of residues and favorable manufacture, and there can furthermore be problems, for example with resistance, there is a constant need to develop novel insecticides which, at least in some areas, have advantages over those of the prior art.

It has been found that compounds of the formula (I), if appropriate also as salts, have a good activity spectrum against animal pests and at the same time good compatibility with plants and favorable toxicological properties with respect to mammals and aquatic animals.

Accordingly, the invention provides amides of the formula (I), and salts thereof 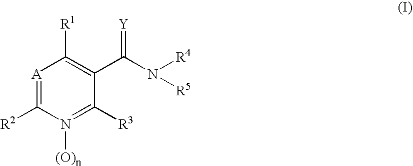
where the symbols and indices are as defined below:
A is CH or N;
Y is O or S;
n is 0 or 1;
R¹ is (C₁-C₄)-haloalkyl;
R², R³ are identical or different and are hydrogen, (C₁-C₄)-alkyl, (C₁-C₄)-haloalkyl or halogen;
R⁴ is hydrogen, (C₁-C₁₀)-alkyl, (C₃-C₁₀)-cycloalkyl, (C₃-C₁₀)-alkenyl or (C₃-C₁₀)-alkynyl, where in the alkyl, cycloalkyl, alkenyl or alkynyl groups mentioned up to three hydrogen atoms may be replaced by halogen, preferably fluorine or chlorine, in the case of fluorine also up to the maximum number;
R⁵ is G¹SR⁶, G¹S(O)R⁷, G¹S(O)₂R⁸, G¹OR⁹, G¹NR¹⁰R¹¹, G²CR¹²—NOR¹³, G¹ON═CR¹⁴R¹⁵ G²CR¹²═N—NR¹⁶R¹⁷, G¹NR¹³N═CR¹⁴R¹⁵, G¹NR¹⁸NR¹⁹R²⁰, G¹ONR²¹R²², G¹NR²³OR²⁴, G²CR¹⁰═N^{(+)(O}⁽⁻⁾)R¹², R²⁵ or G²R²⁶;
G¹ is a straight-chain or branched (C₂-C₆)-alkylene moiety, the distance between the amide nitrogen and the second radical on G¹ being at least C₂, or a (C₃-C₁₀)-cycloalkanediyl group;
G² is a straight-chain or branched (C₁-C₆)-alkylene moiety or a (C₃-C₁₀)-cycloalkanediyl group;
R⁶ is substituted (C₁-C₁₀)-alkyl, unsubstituted or substituted (C₃-C₁₀)-alkenyl, unsubstituted or substituted (C₃-C₁₀)-alkynyl, unsubstituted or substituted (C₃-C₁₀)-cycloalkyl, unsubstituted or substituted (C₄-C₁₀)-cycloalkenyl, substituted aryl or unsubstituted or substituted heterocyclyl or a radical C(NR²⁷)(NR^{27′}R^{27″}), in which R²⁷, R^{27′} and R^{27″} are identical or different and are in each case hydrogen, (C₁-C₁₀)-alkyl or unsubstituted or substituted aryl;
R⁷ is unsubstituted or substituted (C₁-C₁₀)-alkyl, unsubstituted or substituted (C₃-C₁₀)-alkenyl, unsubstituted or substituted (C₃-C₁₀)-alkynyl, unsubstituted or substituted (C₃-C₁₀)-cycloalkyl, unsubstituted or substituted (C₄-C₁₀)-cycloalkenyl, unsubstituted or substituted aryl or unsubstituted or substituted heterocyclyl;
R⁸ is substituted (C₁-C₁₀)-alkyl, unsubstituted or substituted (C₃-C₁₀)-alkenyl, unsubstituted or substituted (C₃-C₁₀)-alkynyl, unsubstituted or substituted (C₃-C₁₀)-cycloalkyl, unsubstituted or substituted (C₄-C₁₀)-cycloalkenyl, unsubstituted or substituted aryl, unsubstituted or substituted heterocyclyl or a group NR²⁸R^{28′}, in which R²⁸ and R^{28′} are identical or different and are in each case hydrogen, unsubstituted or substituted (C₁-C₁₀)-alkyl, unsubstituted or substituted (C₃-C₁₀)-alkenyl, unsubstituted or substituted (C₃-C₁₀)-alkynyl, unsubstituted or substituted (C₃-C₁₀)-cycloalkyl, unsubstituted or substituted aryl or unsubstituted or substituted heterocyclyl and where the different radicals R²⁸ and R^{28′} may be linked to form a 3- to 8-membered ring which may contain a further heteroatom moiety, such as oxygen, sulfur, S(O), S(O)₂ or NR²⁹, where R²⁹ is hydrogen, (C₁-C₁₀)-alkyl unsubstituted or substituted aryl, heterocyclyl, (C₁-C₁₀)-alkylsulfonyl, unsubstituted or substituted aroyl or (C₁-C₁₀)-alkanoyl;
R⁹ is substituted (C₁-C₁₀)-alkyl, unsubstituted or substituted (C₃-C₁₀)-alkenyl, unsubstituted or substituted (C₃-C₁₀)-alkynyl, unsubstituted or substituted (C₃-C₁₀)-cycloalkyl, unsubstituted or substituted (C₄-C₁₀)-cycloalkenyl, substituted aryl, unsubstituted or substituted heterocyclyl, carbamoyl, unsubstituted or substituted mono- or di-(C₁-C₁₀)-alkyl-carbamoyl, where the last-mentioned radical may be cyclically attached and may contain a heteroatom moiety, such as oxygen, sulfur, S(O), S(O)₂ or NR²⁹, unsubstituted or substituted mono- or di-(C₃-C₁₀)-cycloalkylcarbamoyl, unsubstituted or substituted aryl- or unsubstituted or substituted N-aryl-N-(C₁-C₁₀)-alkylcarbamoyl, unsubstituted or substituted (C₁-C₁₀)-alkoxycarbonyl, unsubstituted or substituted (C₃-C₈)-cycloalkyl-(C₁-C₄)-alkoxycarbonyl, unsubstituted or substituted (C₃-C₁₀)-cycloalkoxycarbonyl, unsubstituted or substituted (C₃-C₁₀)-alkenyloxycarbonyl, unsubstituted or substituted (C₃-C₁₀)-alkynyloxycarbonyl, unsubstituted or substituted aryloxycarbonyl or unsubstituted or substituted heterocyclyloxycarbonyl;
R¹⁰ is hydrogen, unsubstituted or substituted (C₁-C₁₀)-alkyl, unsubstituted or substituted (C₃-C₁₀)-alkenyl, unsubstituted or substituted (C₃-C₁₀)-alkynyl, unsubstituted or substituted (C₃-C₁₀)-cycloalkyl, unsubstituted or substituted (C₄-C₁₀)-cycloalkenyl, unsubstituted or substituted aryl or unsubstituted or substituted heterocyclyl;
R¹¹ is substituted (C₁-C₁₀)-alkyl, unsubstituted or substituted (C₃-C₁₀)-alkenyl, unsubstituted or substituted (C₃-C₁₀)-alkynyl, substituted (C₃-C₁₀)-cycloalkyl, unsubstituted or substituted (C₄-C₁₀)-cycloalkenyl, unsubstituted or substituted aryl, unsubstituted or substituted heterocyclyl, unsubstituted or substituted (C₁-C₁₀)-alkanoyl, unsubstituted or substituted (C₃-C₁₀)-alkenoyl, unsubstituted or substituted (C₃-C₁₀)-alkynoyl, unsubstituted or substituted (C₄-C₁₀)-cycloalkanoyl, carbamoyl, unsubstituted or substituted mono- or di-(C₁-C₁₀)-alkylcarbamoyl, where the last-mentioned radical may be cyclically attached and may contain a heteroatom moiety, such as oxygen, sulfur, S(O), S(O)₂ or NR²⁹, unsubstituted or substituted mono- or di-(C₃-C₁₀)-cycloalkylcarbamoyl, unsubstituted or substituted aryl- or unsubstituted or substituted N-aryl-N-(C₁-C₁₀)-alkylcarbamoyl, unsubstituted or substituted (C₁-C₁₀)-alkoxycarbonyl, unsubstituted or substituted (C₃-C₁₀)-alkenyloxycarbonyl, unsubstituted or substituted (C₃-C₁₀)-alkynyloxycarbonyl, unsubstituted or substituted (C₃-C₁₀)-cycloalkoxycarbonyl, unsubstituted or substituted aryloxycarbonyl, unsubstituted or substituted heterocyclyloxycarbonyl, unsubstituted or substituted (C₁-C₁₀)-alkylsulfonyl, unsubstituted or substituted arylsulfonyl, unsubstituted or substituted heterocyclylsulfonyl, unsubstituted benzoyl or benzoyl which is mono- or polysubstituted in the 2-, 4- and/or 6-positions or unsubstituted or substituted naphthoyl;
R¹² has the meanings given above for R¹⁰ or the meaning C₂-alkenyl or C₂-alkynyl; where, if R⁴ and R¹² have the meaning (C₁-C₁₀)-alkyl, these two radicals may be linked to form a five- to eight-membered ring system; and where furthermore, if R¹² has the meaning (C₁-C₁₀)-alkyl, the alkylene moiety G₂ and R¹² may be linked to form a four- to eight-membered ring system which, if chemically possible, may contain, in addition to carbon atoms, a heteroatom moiety, such as oxygen, sulfur, S(O), S(O)₂ or NR²⁹, in the case of oxygen also two not directly adjacent oxygen atoms, and which may also be benzo-fused;
R¹³ is hydrogen, unsubstituted or substituted (C₁-C₁₀)-alkyl, unsubstituted or substituted (C₃-C₁₀)-alkenyl, unsubstituted or substituted (C₃-C₁₀)-alkynyl, unsubstituted or substituted (C₃-C₁₀)-cycloalkyl, unsubstituted or substituted (C₄-C₁₀)-cycloalkenyl, unsubstituted or substituted aryl, unsubstituted or substituted heterocyclyl, unsubstituted or substituted (C₁-C₁₀)-alkanoyl, unsubstituted or substituted (C₃-C₁₀)-alkenoyl, unsubstituted or substituted (C₃-C₁₀)-alkynoyl, unsubstituted or substituted (C₄-C₁₀)-cycloalkanoyl, unsubstituted or substituted aroyl, unsubstituted or substituted heterocyclylcarbonyl, carbamoyl, unsubstituted or substituted mono- or di-(C₁-C₁₀)-alkylcarbamoyl, where the last-mentioned radical may be cyclically attached and may contain a heteroatom moiety, such as oxygen, sulfur, S(O), S(O)₂ or NR²⁹, unsubstituted or substituted mono- or di-(C₃-C₁₀)-cycloalkylcarbamoyl, unsubstituted or substituted aryl- or unsubstituted or substituted N-aryl-N-(C₁-C₁₀)-alkylcarbamoyl, unsubstituted or substituted (C₁-C₁₀)-alkoxycarbonyl, unsubstituted or substituted (C₃-C₁₀)-alkenyloxycarbonyl, unsubstituted or substituted (C₃-C₁₀)-alkynyloxycarbonyl, unsubstituted or substituted (C₃-C₁₀)-cycloalkoxycarbonyl, unsubstituted or substituted aryloxycarbonyl, unsubstituted or substituted heterocyclyloxycarbonyl, unsubstituted or substituted (C₁-C₁₀)-alkylsulfonyl, unsubstituted or substituted arylsulfonyl, unsubstituted or substituted heterocyclylsulfonyl, unsubstituted or substituted aroyl or unsubstituted or substituted heterocyclylcarbonyl;
R¹⁴ and R¹⁵ are identical or different, each having the meaning of R¹⁰, or R¹⁴ and R¹⁵ are linked to form a 3- to 8-membered ring which, in addition to carbon atoms, may contain a heteroatom moiety, such as oxygen, sulfur, S(O), S(O)₂ or NR²⁹, if appropriate incorporating an unsubstituted or substituted benzene ring;
R¹⁶ and R¹⁷ are identical or different, each having the meaning of R¹⁰, or they are linked to form a 3- to 8-membered ring which, in addition to carbon atoms, may contain a heteroatom unit, such as oxygen, sulfur, S(O), S(O)₂ or NR²⁹, or one of the radicals R¹⁶ and R¹⁷ is unsubstituted or substituted (C₁-C₁₀)-alkanoyl, unsubstituted or substituted (C₃-C₁₀)-alkenoyl, unsubstituted or substituted (C₃-C₁₀)-alkynoyl, unsubstituted or substituted (C₄-C₁₀)-cycloalkanoyl, unsubstituted or substituted aroyl, unsubstituted or substituted heterocyclylcarbonyl, carbamoyl, unsubstituted or substituted mono- or di-(C₁-C₁₀)-alkylcarbamoyl, where the last-mentioned radical may be cyclically attached and may contain a heteroatom moiety, such as oxygen, sulfur, S(O), S(O)₂ or NR²⁹, unsubstituted or substituted mono- or di(C₃-C₁₀)-cycloalkylcarbamoyl, unsubstituted or substituted aryl- or unsubstituted or substituted N-aryl-N-(C₁-C₁₀)-alkylcarbamoyl, unsubstituted or substituted (C₁-C₁₀)-alkoxycarbonyl, unsubstituted or substituted (C₃-C₁₀)-alkenyloxycarbonyl, unsubstituted or substituted (C₃-C₁₀)-alkynyloxycarbonyl, unsubstituted or substituted (C₃-C₁₀)-cycloalkoxycarbonyl, unsubstituted or substituted aryloxycarbonyl, unsubstituted or substituted heterocyclyloxycarbonyl, the thiocarbonyl analogs of the abovementioned carbonyl derivatives, unsubstituted or substituted (C₁-C₁₀)-alkylsulfonyl, unsubstituted or substituted arylsulfonyl or unsubstituted or substituted heterocyclylsulfonyl;
R¹⁸, R¹⁹, R²⁰ are identical or different, each having the meaning of R¹⁰, or are linked together to form a 3- to 8-membered ring, which may contain a heteroatom unit, such as oxygen, sulfur, S(O), S(O)₂ or NR²⁹, or R¹⁸ and/or R¹⁹ are/is unsubstituted or substituted (C₁-C₁₀)-alkanoyl, unsubstituted or substituted (C₃-C₁₀)-alkenoyl, unsubstituted or substituted (C₃-C₁₀)-alkynoyl, unsubstituted or substituted (C₄-C₁₀)-cycloalkanoyl, unsubstituted or substituted aroyl, unsubstituted or substituted heterocyclylcarbonyl, carbamoyl, unsubstituted or substituted mono- or di-(C₁-C₁₀)-alkylcarbamoyl, where the last-mentioned radical may also be cyclically attached and may contain a heteroatom unit, such as oxygen, sulfur, S(O) or S(O)₂, unsubstituted or substituted mono- or di-(C₃-C₁₀)-cycloalkylcarbamoyl, unsubstituted or substituted aryl- or unsubstituted or substituted N-aryl-N-(C₁-C₁₀)-alkylcarbamoyl, unsubstituted or substituted (C₁-C₁₀)-alkoxycarbonyl, unsubstituted or substituted (C₃-C₁₀)-alkenyloxycarbonyl, unsubstituted or substituted (C₃-C₁₀)-alkynyloxycarbonyl, unsubstituted or substituted (C₃-C₁₀)-cycloalkoxycarbonyl, unsubstituted or substituted aryloxycarbonyl, unsubstituted or substituted heterocyclyloxycarbonyl, the thiocarbonyl analogs of the abovementioned carbonyl derivatives, unsubstituted or substituted (C₁-C₁₀)-alkylsulfonyl, unsubstituted or substituted arylsulfonyl, unsubstituted or substituted heterocyclylsulfonyl;
R²¹ and R²² are identical or different, each having the meaning of R¹⁰, or they are linked together to form a 3- to 8-membered ring, which may contain a heteroatom moiety, such as oxygen, sulfur, S(O), S(O)₂ or NR²⁹, or one of the radicals R²¹ or R²² is unsubstituted or substituted (C₁-C₁₀)-alkanoyl, unsubstituted or substituted (C₃-C₁₀)-alkenoyl, unsubstituted or substituted (C₃-C₁₀)-alkynoyl, unsubstituted or substituted (C₄-C₁₀)-cycloalkanoyl, unsubstituted or substituted aroyl, unsubstituted or substituted heterocyclylcarbonyl, carbamoyl, unsubstituted or substituted mono- or di-(C₁-C₁₀)-alkylcarbamoyl, where the last-mentioned radical may be cyclically attached and may contain a heteroatom moiety, such as oxygen, sulfur, S(O) or S(O)₂, unsubstituted or substituted mono- or di-(C₃-C₁₀)-cycloalkylcarbamoyl, unsubstituted or substituted aryl- or unsubstituted or substituted N-aryl-N-(C₁-C₁₀)-alkylcarbamoyl, unsubstituted or substituted (C₁-C₁₀)-alkoxycarbonyl, unsubstituted or substituted (C₃-C₁₀)-alkenyloxycarbonyl, unsubstituted or substituted (C₃-C₁₀)-alkynyloxycarbonyl, unsubstituted or substituted (C₃-C₁₀)-cycloalkoxycarbonyl, unsubstituted or substituted aryloxycarbonyl, unsubstituted or substituted heterocyclyloxycarbonyl, unsubstituted or substituted (C₁-C₁₀)-alkylsulfonyl, unsubstituted or substituted arylsulfonyl, unsubstituted or substituted heterocyclylsulfonyl, unsubstituted or substituted aroyl or unsubstituted or substituted heterocyclylcarbonyl;
R²³ and R²⁴ are identical or different, each having the meaning of R¹³, or they are linked together to form a 3- to 8-membered ring;
R²⁵ is unsubstituted or substituted (C₄-C₁₀)-cycloalkenyl, where in this cycloalkenyl group a CH₂ unit may be replaced by a group C═NOR¹³ or a group C═NNR¹⁶R¹⁷;
R²⁶ is unsubstituted or substituted (C₄-C₁₀)-cycloalkenyl or substituted (C₃-C₁₀)-cycloalkyl.

Preferred substituents which may be present, preferably one to three times, in the case of fluorine also up to the maximum number, on the groups mentioned under radicals R⁶ to R²⁶ are:

halogen, cyano, nitro, hydroxyl, thio, amino, (C₁-C₁₀)-alkanoyl, (C₃-C₁₀)-alkenoyl, (C₃-C₁₀)-alkynoyl, (C₄-C₁₀)-cycloalkanoyl, (C₁-C₁₀)-alkoxy, (C₃-C₁₀)-alkenyloxy, (C₃-C₁₀)-alkynyloxy, (C₃-C₁₀)-cycloalkoxy, (C₄-C₁₀)-cycloalkenyloxy, (C₃-C₁₀)-cycloalkyl-(C₁-C₄)-alkoxy, (C₄-C₁₀)-cycloalkenyl-(C₁-C₄)-alkoxy, (C₃-C₁₀)-cycloalkyl-(C₃-C₄)-alkenyloxy, (C₄-C₁₀)-cycloalkenyl-(C₃-C₄)-alkenyloxy, (C₁-C₄)-alkyl-(C₃-C₁₀)-cycloalkoxy, (C₂-C₄)-alkenyl-(C₃-C₁₀)-cycloalkoxy, (C₂-C₄)-alkynyl-(C₃-C₁₀)-cycloalkoxy, (C₁-C₄)-alkyl-(C₄-C₁₀)-cycloalkenyloxy, (C₂-C₄)-alkenyl-(C₄-C₁₀)-cycloalkenyloxy, (C₁-C₄)-alkoxy-(C₁-C₄)-alkoxy, (C₁-C₄)-alkoxy-(C₃-C₄)-alkenyloxy, carbamoyl, mono- or di-(C₁-C₁₀)-alkylcarbamoyl, where the last-mentioned radical may be cyclically attached and may contain a heteroatom moiety, such as oxygen, sulfur, S(O) or S(O)₂, mono- or di-(C₃-C₁₀)-cycloalkylcarbamoyl, (C₁-C₁₀)-alkoxycarbonyl, (C₃-C₁₀)-cycloalkoxycarbonyl, (C₁-C₁₀)-alkanoyloxy, (C₄-C₁₀)-cycloalkanoyloxy, (C₁-C₁₀)-alkanoylamino, (C₃-C₁₀)-alkenoylamino, (C₄-C₁₀)-cycloalkanoylamino, (C₃-C₁₀)-cycloalkyl-(C₁-C₄)-alkanoylamino, the N-(C₁-C₄)-alkylamino analogs of the four last-mentioned radicals, (C₁-C₁₀)-alkylthio, (C₃-C₁₀)-alkenylthio, (C₃-C₁₀)-alkynylthio, (C₃-C₁₀)-cycloalkylthio, (C₄-C₁₀)-cycloalkenylthio, (C₃-C₁₀)-cycloalkyl-(C₁-C₄)-alkylthio, (C₄-C₁₀)-cycloalkenyl-(C₁-C₄)-alkylthio, (C₃-C₁₀)-cycloalkyl-(C₃-C₄)-alkenylthio, (C₄-C₁₀)-cycloalkenyl-(C₃-C₄)-alkenylthio, (C₁-C₄)-alkyl-(C₃-C₁₀)-cycloalkylthio, (C₂-C₄)-alkenyl-(C₃-C₁₀)-cycloalkylthio, (C₂-C₄)-alkynyl-(C₃-C₁₀)-cycloalkylthio, (C₁-C₄)-alkyl-(C₄-C₁₀)-cycloalkenylthio, (C₂-C₄)-alkenyl-(C₄-C₁₀)-cycloalkenylthio, (C₁-C₁₀)-alkylsulfinyl, (C₃-C₁₀)-alkenylsulfinyl, (C₃-C₁₀)-alkynylsulfinyl, (C₃-C₁₀)-cycloalkylsulfinyl, (C₄-C₁₀)-cycloalkenylsulfinyl, (C₃-C₁₀)-cycloalkyl-(C₁-C₄)-alkylsulfinyl, (C₄-C₁₀)-cycloalkenyl-(C₁-C₄)-alkylsulfinyl, (C₃-C₁₀)-cycloalkyl-(C₃-C₄)-alkenylsulfinyl, (C₄-C₁₀)-cycloalkenyl-(C₃-C₄)-alkenylsulfinyl, (C₁-C₄)-alkyl-(C₃-C₁₀)-cycloalkylsulfinyl, (C₂-C₄)-alkenyl-(C₃-C₁₀)-cycloalkylsulfinyl, (C₂-C₄)-alkynyl-(C₃-C₁₀)-cycloalkylsulfinyl, (C₁-C₄)-alkyl-(C₄-C₁₀)-cycloalkenylsulfinyl, (C₂-C₄)-alkenyl-(C₄-C₁₀)-cycloalkenylsulfinyl, (C₂-C₄)-alkynyl-(C₄-C₁₀)-cycloalkenylsulfinyl, (C₁-C₁₀)-alkylsulfonyl, (C₃-C₁₀)-alkenylsulfonyl, (C₃-C₁₀)-alkynylsulfonyl, (C₃-C₁₀)-cycloalkylsulfonyl, (C₄-C₁₀)-cycloalkenylsulfonyl, (C₃-C₁₀)-cycloalkyl-(C₁-C₄)-alkylsulfonyl, (C₄-C₁₀)-cycloalkenyl-(C₁-C₄)-alkylsulfonyl, (C₃-C₁₀)-cycloalkyl-(C₃-C₄)-alkenylsulfonyl, (C₄-C₁₀)-cycloalkenyl-(C₃-C₄)-alkenylsulfonyl, (C₁-C₄)-alkyl-(C₃-C₁₀)-cycloalkylsulfonyl, (C₂-C₄)-alkenyl-(C₃-C₁₀)-cycloalkylsulfonyl, (C₃-C₄)-alkynyl-(C₃-C₁₀)-cycloalkylsulfonyl, (C₁-C₄)-alkyl-(C₄-C₁₀)-cycloalkenylsulfonyl, (C₃-C₄)-alkenyl-(C₄-C₁₀)-cycloalkenylsulfonyl, (C₁-C₁₀)-alkylamino, (C₃-C₁₀)-alkenylamino, (C₃-C₁₀)-alkynylamino, (C₃-C₁₀)-cycloalkylamino, (C₄-C₁₀)-cycloalkenylamino, (C₃-C₁₀)-cycloalkyl-(C₁-C₄)-alkylamino, (C₄-C₁₀)-cycloalkenyl-(C₁-C₄)-alkylamino, (C₃-C₁₀)-cycloalkyl-(C₃-C₄)-alkenylamino, (C₄-C₁₀)-cycloalkenyl-(C₃-C₄)-alkenylamino, (C₁-C₄)-alkyl-(C₃-C₁₀)-cycloalkylamino, (C₂-C₄)-alkenyl-(C₃-C₁₀)-cycloalkylamino, (C₂-C₄)-alkynyl-(C₃-C₁₀)-cycloalkylamino, (C₁-C₄)-alkyl-(C₄-C₁₀)-cycloalkenylamino, (C₂-C₄)-alkenyl-(C₄-C₁₀)-cycloalkenylamino, the N-(C₁-C₄)-alkylamino analogs of the fourteen last-mentioned radicals, (C₁-C₁₀)-trialkylsilyl, (C₃-C₁₀)-cycloalkyl, (C₄-C₁₀)-cycloalkenyl, aryl, aroyl, heterocyclylcarbonyl, aryloxy, arylthio, arylamino, N-(C₁-C₄)-alkyl-N-arylamino, aryl-(C₁-C₄)-alkoxy, aryl-(C₃-C₄)-alkenyloxy, aryl-(C₁-C₄)-alkylthio, aryl-(C₃-C₄)-alkenylthio, aryl-(C₁-C₄)-alkylamino, N-(C₁-C₄)-alkyl-N-aryl-(C₁-C₄)-alkylamino, aryl-(C₃-C₄)-alkenylamino, N-(C₁-C₄)-alkyl-N-aryl-(C₃-C₄)-alkenylamino, arylcarbamoyl, N-aryl-N-(C₁-C₄)-alkylcarbamoyl, aryl-(C₁-C₈)-dialkylsilyl, diaryl-(C₁-C₈)-alkylsilyl, triarylsilyl and 5- or 6-membered heterocyclyl, where the cyclic moiety of the 21 last-mentioned radicals is unsubstituted or substituted by one or more radicals from the group consisting of halogen, cyano, nitro, amino, hydroxyl, thio, (C₁-C₄)-alkyl, (C₁-C₄)-haloalkyl, (C₃-C₈)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, halo-(C₁-C₄)-alkylsulfonyl, (C₁-C₄)-alkylamino, trimethylsilyl and (C₁-C₄)-alkanoyl; where in the substituents mentioned above which may be present on the radicals R⁶ to R²⁶, hydrogen atoms attached to carbon may be replaced by up to three halogen atoms, in the case of fluorine also up to the maximum number.

The symbols and indices in formula (I) are preferably as defined below:
A is preferably CH.
Y is preferably O.
n is preferably 0.
R¹ is preferably (C₁-C₄)-alkyl, which is mono- or polysubstituted by F and/or Cl, in particular CF₃, CHF₂ or CF₂Cl.
R², R³ are preferably hydrogen.
R⁴ is preferably hydrogen or (C₁-C₄)-alkyl.
G¹ is preferably a straight-chain or branched (C₂-C₄)-alkylene unit, the distance between the amide nitrogen and the second radical on G¹ being at least C₂.
G² is preferably a straight-chain or branched (C₁-C₄)-alkylene unit.
R⁵ is preferably G¹SR⁶, G¹S(O)R⁷, G¹S(O)₂R⁸, G¹OR⁹, G¹NR¹⁰R¹¹, G²CR¹²═NOR¹³, G¹ON═CR¹⁴ R¹⁵, G²CR¹²═NR¹⁶ R¹⁷ or G²CR¹⁰═N⁽⁺⁾(O⁽⁻⁾)R¹².

The symbols and indices in formula (I) are particularly preferably as defined below:
A is CH.
Y is O.
n is 0.
R¹ is CF₃.
R² and R³ are hydrogen.
R⁴ is hydrogen or methyl.
G¹ is CH₂—CH₂, CH₂—CH₂—CH₂, CH(CH₃)—CH₂ or CH(C₂H₅)—CH₂.
G² is one of the groups mentioned for G¹ and CH₂ or CH(CH₃).

If R⁵ is G¹SR⁶ or G¹S(O)₂R⁸, R⁶ or R⁸ is

preferably (C₁-C₄)-fluoroalkyl, (C₁-C₄)-alkyl which is substituted by unsubstituted or substituted phenyl, furyl, thienyl or by (C₃-C₆)-cycloalkyl, (C₃-C₆)-alkenyl, (C₃-C₆)-haloalkenyl, (C₃-C₆)-alkynyl, (C₃-C₆)-cycloalkyl or unsubstituted or substituted phenyl or heteroaryl, preferred substituents on the phenyl or heteroaryl group being fluorine, chlorine, methyl, trifluoromethyl and methoxy and heteroaryl preferably being thienyl, pyridyl, pyrimidyl, thiazolyl, benzothiazolyl, imidazolyl, benzimidazolyl, thiadiazolyl or tetrazolyl.

If R⁵ is G¹S(O)R⁷, R⁷ is preferably (C₁-C₈)-alkyl, (C₁-C₄)-fluoroalkyl, (C₁-C₄)-alkyl which is substituted by unsubstituted or substituted phenyl, furyl, thienyl or by (C₃-C₆)-cycloalkyl, (C₃-C₆)-alkenyl, (C₃-C₆)-haloalkenyl, (C₃-C₆)-alkynyl, (C₃-C₆)-cycloalkyl or unsubstituted or substituted phenyl or heteroaryl, preferred substituents on the phenyl or heteroaryl group being, in particular, fluorine, chlorine, methyl, trifluoromethyl and methoxy and heteroaryl preferably being thienyl, pyridyl, pyrimidyl, thiazolyl, benzothiazolyl, imidazolyl, benzimidazolyl, thiadiazolyl or tetrazolyl.

If R⁵ is G¹OR⁹, R⁹ is preferably (C₁-C₄)-fluoroalkyl, (C₃-C₆)-alkenyl, (C₃-C₆)-alkynyl or (C₃-C₆)-cycloalkyl.

If R⁵ is G¹NR¹⁰R¹¹, R¹⁰ is preferably hydrogen or (C₁-C₄)-alkyl, in particular methyl, and R¹¹ is preferably (C₁-C₈)-alkoxycarbonyl, (C₄-C₆)-cycloalkoxycarbonyl or unsubstituted or halogen-, (C₁-C₄)-alkyl- or trifluoromethyl-substituted heteroaryl, in particular pyridyl or pyrimidyl. With very particular preference, R¹¹ is (C₁-C₄)-alkoxycarbonyl.

If R⁵ is G²CR¹²═NOR¹³, R¹² is preferably hydrogen or (C₁-C₄)-alkyl and R¹³ is preferably unsubstituted or substituted (C₁-C₈)-alkyl, (C₃-C₆)-alkenyl, (C₃-C₆)-alkynyl, (C₃-C₆)-cycloalkyl, unsubstituted or fluorine-, chlorine-, methyl-, methoxy- or trifluormethyl-substituted benzyl, thienylmethyl or furfuryl; with very particular preference, R¹³ is (C₁-C₄)-alkyl, (C₃-C₆)-alkenyl, unsubstituted or fluorine- or chlorine-substituted benzyl or thienylmethyl.

If R⁵ is G¹ON═CR¹⁴R¹⁵, R¹⁴ and R¹⁵ are preferably hydrogen, (C₁-C₈)-alkyl, (C₃-C₆)— cycloalkyl, unsubstituted or fluorine-, chlorine-, methyl-, methoxy- or trifluoromethyl-substituted phenyl or pyridyl, R¹⁴ and R¹⁵ are linked to form a 4- to 6-membered carbocyclic ring.

If R⁵ is G²CR¹²═N—NR¹⁶R¹⁷, R¹² is preferably hydrogen or (C₁-C₄)-alkyl, preferably methyl, R¹⁶ is preferably hydrogen or (C₁-C₄)-alkyl, preferably methyl, and R¹⁷ is preferably unsubstituted or chlorine- and/or trifluoromethyl-substituted phenyl or pyridyl, (C₁-C₈)-alkanoyl, (C₄-C₇)-cycloalkanoyl, unsubstituted or fluorine-, chlorine-methyl-, methoxy- or trifluoromethyl-substituted aroyl, pyridylcarbonyl, furoyl, carbamoyl, mono- or di-(C₁-C₄)-alkylcarbamoyl, (C₁-C₄)-alkoxycarbonyl, or the thiocarbonyl analog of one of the carbonyl derivatives mentioned above.

If R⁵ is G²CR¹⁰═N⁽⁺⁾(O⁽⁻⁾)R¹², R¹⁰ is preferably hydrogen or (C₁-C₄)-alkyl and R¹² is preferably (C₁-C₈)-alkyl or (C₅-C₆)-cycloalkyl.

If R⁵ is G¹R²⁶, R²⁶ is preferably fluoro-(C₃-C₆)-cycloalkyl or (C₁-C₄)-alkyl-(C₃-C₆)-cycloalkyl.

Particular preference is given to the following groups of compounds of the formulae (Ia) to (Io): 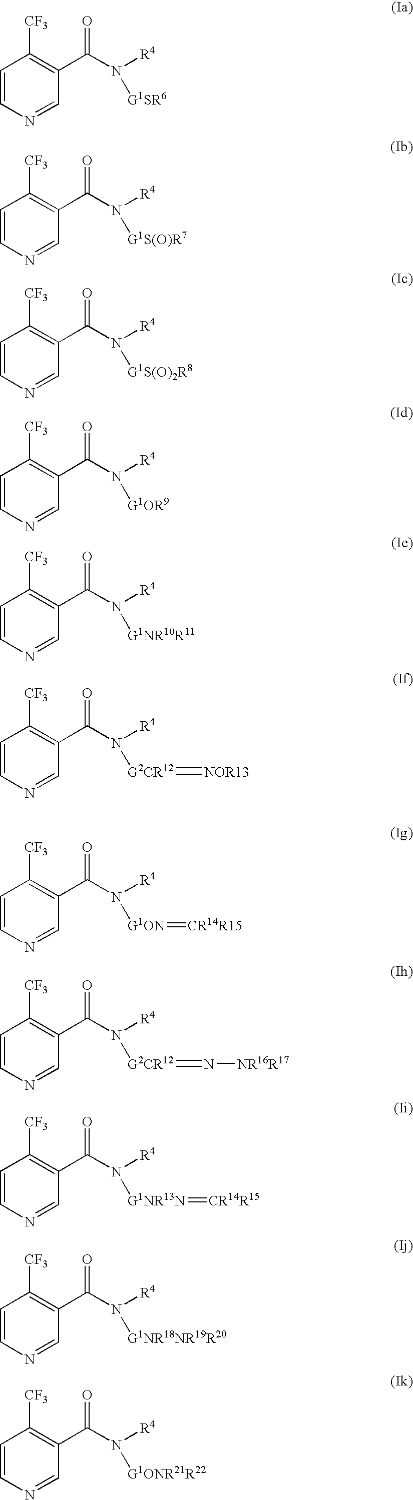 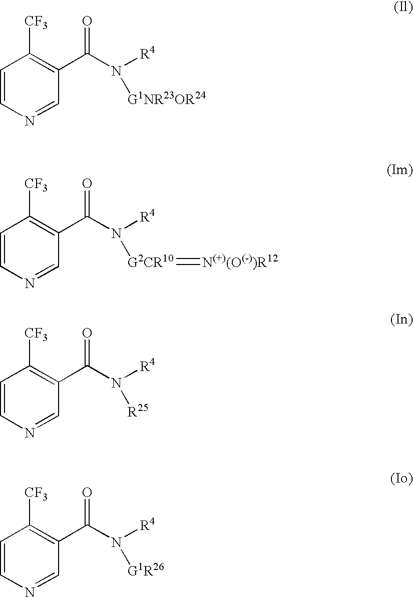
where the symbols and indices have the meanings and preferred meanings given above.

The term “(C₂-C₆)-alkylene unit” embraces, for example, the groups (CH₂)₂, CH(CH₃), (CH₂)₃, (CH₂)₄, CH(CH₃)CH₂, CH(C₂H₅), CH₂CH(CH₃), CH₂CH(CH₃)CH₂, CH(CH₃)CH₂CH₂ and CH₂CH₂CH(CH₃), (CH₂)₅, CH₂C(CH₃)₂CH₂ or (CH₂)₆. The term “(C₁-C₆)-alkylene unit” embraces the groups listed above and the methylene group.

The term “halogen” embraces fluorine, chlorine, bromine and iodine.

“(C₁-C₄)-Alkyl” is a straight-chain or branched hydrocarbon radical having 1, 2, 3 or 4 carbon atoms, for example the methyl, ethyl, propyl, isopropyl, 1-butyl, 2-butyl, isobutyl or tert-butyl radical.

Correspondingly, an alkyl radical having a larger range of carbon atoms is to be understood as meaning a straight-chain or branched saturated hydrocarbon radical which comprises a number of carbon atoms corresponding to the stated range. Accordingly, the term “(C₁-C₁₀)-alkyl” embraces the alkyl radicals mentioned above and also, for example, the pentyl, 2-methylbutyl, 1,1-dimethylpropyl, hexyl, heptyl, octyl, tert-octyl, nonyl or decyl radical.

“(C₁-C₄)-Haloalkyl” is an alkyl group mentioned under “(C₁-C₄)-alkyl” in which one or more hydrogen atoms are replaced by the same number of identical or different halogen atoms, preferably by chlorine or fluorine, for example, the mono-, di- or trifluoromethyl group, the 1- or 2-fluoroethyl, the 2,2,2-trifluoroethyl, the chloromethyl, the trichloromethyl or the 1,1,2,2-tetrafluoroethyl group.

“Alkenyl” and “alkynyl” with a prefix in which the number of carbon atoms is stated are a straight-chain or branched hydrocarbon radical having a number of carbons corresponding to the stated range and comprising at least one multiple bond, which may be in any position of the unsaturated radical in question. Accordingly, “(C₃-C₁₀)-alkenyl” is the allyl, 2-methylpropenyl, 1- or 2-butenyl, pentenyl, 2-methylpentenyl, hexenyl, heptenyl, octenyl, nonenyl or decenyl group.

“(C₂-C₁₀)-Alkenyl” is the abovementioned groups and also the vinyl group.

“(C₃-C₁₀)-Alkynyl” is, for example, the propargyl, 2-methylpropynyl, 2-butynyl, pentynyl, 2-methylpentynyl, hexynyl, heptynyl, octynyl, nonynyl or the decynyl group.

“(C₂-C₁₀)-Alkynyl” is to be understood as meaning the abovementioned radicals and also the ethynyl group.

“(C₃-C₁₀)-Cycloalkyl” is monocyclic alkyl radicals, such as the cyclopropyl, cyclobutyl, cyclopentyl, cyclohexyl, cycloheptyl, cyclooctyl or cyclodecyl radical, is bicyclic alkyl radicals, such as the norbornyl or bicyclo[2.2.2]octyl radical, or is fused systems, such as the decahydronaphthyl radical.

“(C₄-C₁₀)-Cycloalkenyl” is monocyclic cycloalkenyl radicals, such as the cyclobutenyl, cyclopentenyl, cyclohexenyl, cycloheptenyl, cyclooctenyl or cyclodecenyl radical, is bicyclic alkenyl radicals, such as the norbornenyl or bicyclo[2.2.2]octenyl radical, or is fused systems, such as the tetra-, hexa- or octahydronaphthyl radical.

“(C₁-C₁₀)-Alkanoyl” is, for example, the formyl, acetyl, propionyl, butyryl, 2-methylbutyryl, pivaloyl, octanoyl or decanoyl group.

“(C₃-C₁₀)-Alkenoyl” is, for example, the acryl, methacryl, crotonoyl, dimethylacryl or octenoyl group.

“(C₃-C₁₀)-Alkynoyl” is, for example, the propynoyl, butynoyl, hexynoyl or octynoyl group.

“(C₄-C₁₀)-Cycloalkanoyl” is, for example, the cyclopropylcarbonyl, cyclobutylcarbonyl, cyclopentylcarbonyl, cyclohexylcarbonyl or cyclooctylcarbonyl.

“(C₁-C₄)-Alkoxy” and “(C₁-C₁₀)-alkoxy” are alkoxy groups whose hydrocarbon radicals have the meanings given under the terms “(C₁-C₄)-alkyl” and “(C₁-C₁₀)-alkyl”.

“(C₃-C₁₀)-Alkenyloxy”, “(C₃-C₁₀)-alkynyloxy”, “(C₃-C₁₀)-cycloalkoxy” and “(C₄-C₁₀)-cycloalkenyloxy” are ether groups whose hydrocarbon radicals have the meanings given under the term “(C₃-C₁₀)-alkenyl”, “(C₃-C₁₀)-alkynyl”, “(C₃-C₁₀)-cycloalkyl” and “(C₄-C₁₀)-cycloalkenyl”.

“(C₃-C₁₀)-Cycloalkyl-(C₁-C₄)-alkoxy” is, for example the cyclopropylmethoxy, cyclopropylethoxy, cyclobutylmethoxy, cyclopentylmethoxy, cyclohexylmethoxy or the cyclohexylethoxy group.

“(C₄-C₁₀)-Cycloalkenyl-(C₁-C₄)-alkoxy” is, for example, the cyclobutenylmethoxy, cyclopentenylmethoxy, cyclohexenylmethoxy or the cyclohexenylethoxy group.

“(C₃-C₁₀)-Cycloalkyl-(C₃-C₄)-alkenyloxy” is, for example, the cyclopropylallyloxy, cyclobutylallyloxy or the cyclopentylallyloxy group.

“(C₄-C₁₀)-Cycloalkenyl-(C₃-C₄)-alkenyloxy” is, for example, the cyclobutenylallyloxy or the cyclopentenylallyloxy group.

“(C₁-C₄)-Alkyl-(C₃-C₁₀)-cycloalkoxy” is, for example, the methylcyclopentyloxy, ethylcyclopentyloxy, methylcyclohexyloxy or the ethylcyclohexyloxy group.

“(C₂-C₄)-Alkenyl-(C₃-C₁₀)-cycloalkoxy” is, for example, the vinylcyclopentyloxy, allylcyclopentyloxy, vinylcyclohexyloxy or the allylcyclohexyloxy group.

“(C₂-C₄)-Alkynyl-(C₃-C₁₀)-cycloalkoxy” is, for example, the ethynylcyclopentyloxy, propynylcyclopentyloxy, ethynylcyclohexyloxy or the propynylcyclohexyloxy group.

“(C₁-C₄)-Alkyl-(C₄-C₁₀)-cycloalkenyloxy” is, for example, the methylcyclopentenyloxy, ethylcyclopentenyloxy, methylcyclohexenyloxy or the ethylcyclohexenyloxy group.

“(C₂-C₄)-Alkenyl-(C₃-C₁₀)-cycloalkenyloxy” is, for example, the vinylcyclopentenyloxy, allylcyclopentenyloxy, vinylcyclohexenyloxy or the allylcyclohexenyloxy group.

“(C₁-C₄)-Alkoxy-(C₁-C₄)-alkoxy” is an alkoxy group as defined above which is substituted by a further alkoxy group, such as the ethoxymethoxy, 1-methoxyethoxy, 1-ethoxyethoxy or the 1-methoxypropoxy group.

“(C₁-C₄)-Alkoxy-(C₃-C₄)-alkenyloxy” is, for example, the methoxyallyloxy or the ethoxyallyloxy group.

“Mono- or di-(C₁-C₁₀)-alkylcarbamoyl, where the last-mentioned radical may be cyclically attached and may contain a heteroatom moiety, such as oxygen, sulfur, S(O) or S(O)₂” is, for example, the methyl-, ethyl-, propyl-, isopropyl-, butyl- or tert-butylcarbamoyl group or the dimethyl-, diethyl-, methylethyl- or diisopropylcarbamoyl group, but also cyclic derivatives, such as the pyrrolidino-, morpholino-, thiomorpholino- or piperidinocarbamoyl group.

“Mono- or di-(C₃-C₁₀)-cycloalkylcarbamoyl” is, for example, the cyclopropyl-, cyclobutyl-, cyclopentyl- or cyclohexylcarbamoyl group or the dicyclopropyl-, dicyclobutyl-, dicyclopentyl- or dicyclohexylcarbamoyl group.

“(C₁-C₁₀)-Alkoxycarbonyl” is, for example, the methoxycarbonyl, ethoxycarbonyl, propoxycarbonyl, isopropoxycarbonyl, butoxycarbonyl, isobutoxycarbonyl, sec-butoxycarbonyl or the tert-butoxycarbonyl group.

“(C₃-C₁₀)-Cycloalkoxycarbonyl” is, for example, the cyclopropoxycarbonyl, cyclobutoxycarbonyl, cyclopentyloxycarbonyl or the cyclohexyloxycarbonyl group.

“(C₁-C₁₀)-Alkanoyloxy” is, for example, the acetoxy, propionyloxy, butanoyloxy or the pivaloyloxy group.

“(C₄-C₁₀)-Cycloalkanoyloxy” is, for example, the cyclopropylcarbonyloxy, cyclobutylcarbonyloxy, cyclopentylcarbonyloxy or the cyclohexylcarbonyloxy group.

“(C₁-C₁₀)-Alkanoylamino” is, for example the formylamino, acetylamino, propionylamino, isopropionylamino, butanoylamino or the pivaloylamino group.

“(C₃-C₁₀)-Alkenoylamino” is, for example, the acrylamino, methacrylamino, dimethylacrylamino or the crotonylamino group.

“(C₄-C₁₀)-Cycloalkanoylamino” is, for example, the cyclopropanoylamino, cyclobutanoylamino, cyclopentanoylamino or the cyclohexanoylamino group.

“(C₃-C₁₀)-Cycloalkyl-(C₁-C₄)-alkanoylamino” is, for example, the cyclopropylacetylamino or the cyclopentylacetylamino group.

“(C₁-C₁₀)-Alkylthio” is an alkylthio group whose hydrocarbon radical has the meaning given under the term “(C₁-C₁₀)-alkyl”.

“(C₃-C₁₀)-Alkenylthio” is an alkenylthio group whose hydrocarbon radical has the meaning given under the term “(C₃-C₁₀)-alkenyl”.

“(C₃-C₁₀)-Alkynylthio” is an alkynylthio group whose hydrocarbon radical has the meaning given under the term “(C₃-C₁₀)-alkynyl”.

“(C₃-C₁₀)-Cycloalkylthio” is a cycloalkylthio group whose hydrocarbon radical has the meaning given under the term “(C₃-C₁₀)-cycloalkyl”.

“(C₄-C₁₀)-Cycloalkenylthio” is a cycloalkenylthio group whose hydrocarbon radical has the meaning given under the term “(C₄-C₁₀)-cycloalkenyl”.

“(C₃-C₁₀)-Cycloalkyl-(C₁-C₄)-alkylthio” is, for example, the cyclopropylmethylthio, cyclopropylethylthio, cyclopentylmethylthio or the cyclohexylmethylthio group.

“(C₄-C₁₀)-Cycloalkenyl-(C₁-C₄)-alkylthio” is, for example, the cyclopentenylmethylthio or the cyclohexenylmethylthio group.

“(C₃-C₁₀)-Cycloalkyl-(C₃-C₄)-alkenylthio” is, for example, the cyclopropylallylthio, cyclopentylallylthio or the cyclohexylallylthio group.

“(C₄-C₁₀)-Cycloalkenyl-(C₃-C₄)-alkenylthio” is, for example, the cyclopentenylallylthio or the cyclohexenylallylthio group.

“(C₁-C₄)-Alkyl-(C₃-C₁₀)-cycloalkylthio” is, for example, the methylcyclopentylthio or the methylcyclohexylthio group.

“(C₁-C₄)-Alkyl-(C₄-C₁₀)-cycloalkenylthio” is, for example, the methylcyclopentenylthio or the methylcyclohexenylthio group.

“(C₂-C₄)-Alkenyl-(C₃-C₁₀)-cycloalkylthio” is, for example, the vinylcyclopentylthio, allylcyclopentylthio, vinylcyclohexylthio or the allylcyclohexylthio group.

“(C₂-C₄)-Alkynyl-(C₃-C₁₀)-cycloalkylthio” is, for example, the ethynylcyclopentylthio, propargylcyclopentylthio, ethynylcyclohexylthio or the propargylcyclohexylthio group.

“(C₁-C₄)-Alkyl-(C₄-C₁₀)-cycloalkenylthio” is, for example, the methylcyclopentenylthio or the methylcyclohexenylthio group.

“(C₂-C₄)-Alkenyl-(C₄-C₈)-cycloalkenylthio” is, for example, the allylcyclopentenylthio or the allylcyclohexenylthio group.

“(C₁-C₁₀)-Alkylsulfinyl” is, for example, the methyl, ethyl, propyl, isopropyl, butyl, isobutyl, sec-butyl, tert-butyl or octylsulfinyl group.

“(C₃-C₁₀)-Alkenylsulfinyl” is, for example, the allyl, methylallyl, butenyl or octenylsulfinyl group.

“(C₃-C₁₀)-Alkynylsulfinyl” is, for example, the propargyl, butynyl or octynylsulfinyl group.

“(C₃-C₁₀)-Cycloalkylsulfinyl” is a cycloalkylsulfinyl group whose hydrocarbon radical has the meaning given under the term “(C₃-C₁₀)-cycloalkyl”.

“(C₄-C₁₀)-Cycloalkenylsulfinyl” is a cycloalkenylsulfinyl group whose hydrocarbon radical has the meaning given under the term “(C₄-C₁₀)-cycloalkenyl”.

“(C₃-C₁₀)-Cycloalkyl-(C₁-C₄)-alkylsulfinyl” is, for example, the cyclopropylmethylsulfinyl, cyclopropylethylsulfinyl, cyclopentylmethylsulfinyl or the cyclohexylmethylsulfinyl group.

“(C₄-C₁₀)-Cycloalkenyl-(C₁-C₄)-alkylsulfinyl” is, for example, the cyclopentenylmethylsulfinyl or the cyclohexenylmethylsulfinyl group.

“(C₃-C₁₀)-Cycloalkyl-(C₃-C₄)-alkenylsulfinyl” is, for example, the cyclopropylallylsulfinyl, cyclopentylallylsulfinyl or the cyclohexylallylsulfinyl group.

“(C₄-C₁₀)-Cycloalkenyl-(C₃-C₄)-alkenylsulfinyl” is, for example, the cyclopentenylallylsulfinyl or the cyclohexenylallylsulfinyl group.

“(C₁-C₄)-Alkyl-(C₃-C₁₀)-cycloalkylsulfinyl” is, for example, the methylcyclopentylsulfinyl or the methylcyclohexylsulfinyl group.

“(C₁-C₈)-Alkyl-(C₄-C₁₀)-cycloalkenylsulfinyl” is, for example, the methylcyclopentenylsulfinyl or the methylcyclohexenylsulfinyl group.

“(C₂-C₄)-Alkenyl-(C₃-C₁₀)-cycloalkylsulfinyl” is, for example, the vinylcyclopentylsulfinyl, allylcyclopentylsulfinyl, vinylcyclohexylsulfinyl or the allylcyclohexylsulfinyl group.

“(C₂-C₄)-Alkynyl-(C₃-C₁₀)-cycloalkylsulfinyl” is, for example, the ethynylcyclopentylsulfinyl, propargylcyclopentylsulfinyl, ethynylcyclohexylsulfinyl or the propargylcyclohexylsulfinyl group.

“(C₂-C₄)-Alkenyl-(C₄-C₁₀)-cycloalkenylsulfinyl” is, for example, the vinylcyclopentenylsulfinyl, allylcyclopentenylsulfinyl, vinylcyclohexenylsulfinyl or the allylcyclohexenylsulfinyl group.

“(C₂-C₄)-Alkynyl-(C₄-C₁₀)-cycloalkenylsulfinyl” is, for example, the ethynylcyclopentenylsulfinyl, propargylcyclopentenylsulfinyl, ethynylcyclohexenylsulfinyl or the propargylcyclohexenylsulfinyl group.

“(C₁-C₁₀)-Alkylsulfonyl” is, for example, the methyl, ethyl, propyl, isopropyl, butyl, isobutyl, sec-butyl, tert-butyl or octylsulfonyl group.

“(C₃-C₁₀)-Alkenylsulfonyl” is, for example, the allyl, methylallyl, butenyl or octenylsulfonyl group.

“(C₃-C₁₀)-Alkynylsulfonyl” is, for example, the propargyl, butynyl or octynylsulfonyl group.

“(C₃-C₁₀)-Cycloalkylsulfonyl” is a cycloalkylsulfonyl group whose hydrocarbon radical has the meaning given under the term “(C₃-C₁₀)-cycloalkyl”.

“(C₄-C₁₀)-Cycloalkenylsulfonyl” is a cycloalkenylsulfonyl group whose hydrocarbon radical has the meaning given under the term “(C₄-C₁₀)-cycloalkenyl”.

“(C₃-C₁₀)-Cycloalkyl-(C₁-C₄)-alkylsulfonyl” is, for example, the cyclopropylmethylsulfonyl, cyclopropylethylsulfonyl, cyclopentylmethylsulfonyl or the cyclohexylmethylsulfonyl group.

“(C₄-C₁₀)-Cycloalkenyl-(C₁-C₄)-alkylsulfonyl” is, for example, the cyclopentenylmethylsulfonyl or the cyclohexenylmethylsulfonyl group.

“(C₃-C₁₀)-Cycloalkyl-(C₃-C₄)-alkenylsulfonyl” is, for example, the cyclopropylallylsulfonyl, cyclopentylallylsulfonyl or the cyclohexylallylsulfonyl group.

“(C₄-C₁₀)-Cycloalkenyl-(C₃-C₄)-alkenylsulfonyl” is, for example, the cyclopentenylallylsulfonyl or the cyclohexenylallylsulfonyl group.

“(C₁-C₄)-Alkyl-(C₃-C₁₀)-cycloalkylsulfonyl” is, for example, the methylcyclopentylsulfonyl or the methylcyclohexylsulfonyl group.

“(C₁-C₄)-Alkyl-(C₄-C₁₀)-cycloalkenylsulfonyl” is, for example, the methycyclopentenylsulfonyl or the methylcyclohexenylsulfonyl group.

“(C₂-C₄)-Alkenyl-(C₃-C₁₀)-cycloalkylsulfonyl” is, for example, the vinylcyclopentylsulfonyl, allylcyclopentylsulfonyl, vinylcyclohexylsulfonyl or the allylcyclohexylsulfonyl group.

“(C₂-C₄)-Alkynyl-(C₃-C₁₀)-cycloalkylsulfonyl” is, for example, the ethynylcyclopentylsulfonyl, propargylcyclopentylsulfonyl, ethynylcyclohexylsulfonyl or the propargylcyclohexylsulfonyl group.

“(C₂-C₄)-Alkenyl-(C₄-C₁₀)-cycloalkenylsulfonyl” is, for example, the vinylcyclopentenylsulfonyl, allylcyclopentenylsulfonyl, vinylcyclohexenylsulfonyl or the allylcyclohexenylsulfonyl group.

“(C₁-C₁₀)-Alkylamino” is an amino group whose hydrocarbon radical has the meaning given under the term “(C₁-C₁₀)-alkyl”.

“(C₃-C₁₀)-Alkenylamino” is an amino group whose hydrocarbon radical has the meaning given under the term “(C₁-C₁₀)-alkenyl”.

“(C₃-C₁₀)-Alkynylamino” is an amino group whose hydrocarbon radical has the meaning given under the term “(C₃-C₁₀)-alkynyl”.

“(C₃-C₁₀)-Cycloalkylamino” is an amino group whose hydrocarbon radical has the meaning given under the term “(C₃-C₁₀)-cycloalkyl”.

“(C₃-C₁₀)-Cycloalkenylamino” is an amino group whose hydrocarbon radical has the meaning given under the term “(C₃-C₁₀)-cycloalkenyl”.

“(C₃-C₁₀)-Cycloalkyl-(C₁-C₄)-alkylamino” is, for example, the cyclopropylmethylamino, cyclopropylethylamino, cyclopentylmethylamino or the cyclohexylmethylamino group.

“(C₄-C₁₀)-Cycloalkenyl-(C₁-C₄)-alkylamino” is, for example, the cyclopentenylmethylamino or the cyclohexenylmethylamino group.

“(C₄-C₁₀)-Cycloalkyl-(C₃-C₄)-alkenylamino” is, for example, the cyclopropylallylamino, cyclopentylallylamino or the cyclohexylallylamino group.

“(C₄-C₁₀)-Cycloalkenyl-(C₃-C₄)-alkenylamino” is, for example, the cyclopentenylallylamino or the cyclohexenylallylamino group.

“(C₁-C₄)-Alkyl-(C₃-C₁₀)-cycloalkylamino” is, for example, the methylcyclopentylamino or the methylcyclohexylamino group.

“(C₁-C₄)-Alkyl-(C₄-C₁₀)-cycloalkenylamino” is, for example, the methylcyclopentenylamino or the methylcyclohexenylaminc group.

“(C₂-C₄)-Alkenyl-(C₃-C₁₀)-cycloalkylamino”¹ is, for example, the vinylcyclopentylamino, allylcyclopentylamino, vinylcyclohexylamino or the allylcyclohexylamino group.

“(C₂-C₄)-Alkynyl-(C₃-C₁₀)-cycloalkylamino” is, for example, the ethynylcyclopentylamino, propargylcyclopentylamino, ethynylcyclohexylamino or the propargylcyclohexylamino group.

“(C₂-C₄)-Alkenyl-(C₄-C₁₀)-cycloalkenylamino” is, for example, the vinylcyclopentenylamino, allylcyclopentenylamino, vinylcyclohexenylamino or the allylcyclohexenylamino group.

“(C₁-C₁₀)-Trialkylsilyl” is a silicon atom which carries three identical or different alkyl radicals according to the above definition.

“Aryl” is a carbocyclic, i.e. constructed of carbon atoms, aromatic radical having preferably 6 to 14, in particular 6 to 12, carbon atoms, such as phenyl, naphthyl or biphenylyl, preferably phenyl.

“Aroyl” is an aryl radical as defined above which is attached via a carbonyl group, for example the benzoyl group.

“Heterocyclyl” preferably is a cyclic radical which may be fully saturated, partially unsaturated or fully unsaturated and which may be interrupted by at least one or more identical or different atoms from the group consisting of nitrogen, sulfur and oxygen, where, however, two oxygen atoms may not be directly adjacent and at least one carbon atom has to be present in the ring, for example a thiophene, furan, pyrrole, thiazole, oxazole, imidazole, isothiazole, isoxazole, pyrazole, 1,3,4-oxadiazole, 1,3,4-thiadiazole, 1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benzo[c]thiophene, benzo[c]furan, isoindole, benzoxazole, benzothiazole, benzimidazole, benzisoxazole, benzisothiazole, benzopyrazole, benzothiadiazole, benzotriazole, dibenzofuran, dibenzothiophene, carbazole, pyridine, pyrazine, pyrimidine, pyridazine, 1,3,5-triazine, 1,2,4-triazine, 1,2,4,5-tetrazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine, 4H-quinolizine, piperidine, morpholine, piperazine, oxetane, oxirane, pyrrolidine, oxazoline, tetrahydrofuran, tetrahydropyran, isoxazolidine or thiazolidine.

Among the groups mentioned under “heterocyclyl”, “heteroaryl” is in each case the fully unsaturated aromatic heterocyclic compounds, for example pyridine, pyrimidine, furan, thiophene, oxazole, thiazole, imidazole, pyrazole, isoxazole, 1,2,4-triazole, tetrazole, pyrazine or pyridazine.

Particularly preferably, heterocyclyl is a saturated, partially saturated or aromatic ring system having 3 to 6 ring members and 1 to 4 heteroatoms from the group consisting of O, S and N, where at least one carbon atom has to be present in the ring.

Very particularly preferably, heterocyclyl is a pyridine, pyrimidine, (1,2,4)-oxadiazole, (1,3,4)-oxadiazole, pyrrole, furan, thiophene, oxazole, thiazole, imidazole, pyrazole, isoxazole, 1,2,4-triazole, tetrazole, pyrazine, pyridazine, oxazoline, thiazoline, tetrahydrofuran, tetrahydropyran, morpholine, piperidine, piperazine, pyrroline, pyrrolidine, oxazolidine, thiazolidine, oxirane or oxetane radical.

“Aryloxy” is an aryl radical as defined above which is attached via an oxygen atom, for example the phenoxy or naphthyloxy group.

“Arylthio” is an aryl radical attached via a sulfur atom, for example, the phenylthio- or the 1- or 2-naphthylthio radical.

“Arylamino” is an aryl radical which is attached via a nitrogen atom, for example the anilino or 1- or 2-naphthylamino radical.

“N-(C₁-C₄)-Alkylarylamino is, for example, the N-methyl- or N-ethylanilino radical.

“Aryl-(C₁-C₄)-alkoxy” is an aryl radical which is attached via a (C₁-C₄)-alkoxy group, for example, the benzyloxy, phenylethoxy, phenylbutoxy or naphthylmethoxy radical.

“Aryl-(C₃-C₄)-alkenyloxy” is an aryl radical which is attached via a (C₃-C₄)-alkenyloxy group, for example the 1-, 2- or 3-phenylallyloxy radical.

“Aryl-(C₁-C₄)-alkylthio” is an aryl radical which is attached via an alkylthio radical, for example the benzylthio, naphthylmethylthio or the 1- or 2-phenylethylthio radical.

“Aryl-(C₃-C₄)-alkenylthio” is an aryl radical attached via a (C₃-C₄)-alkenylthio group, for example the 1-, 2- or 3-phenylallylthio radical.

“Aryl-(C₁-C₄)-alkylamino” is an aryl radical attached via a (C₁-C₄)-alkylamino group, for example the benzylamino, naphthylamino, the 1- or 2-phenylethylamino or the 3-phenylpropylamino radical.

“N-(C₁-C₄)-Alkyl-N-aryl-(C₁-C₄)-alkylamino” is, for example, the N-methyl-N-benzylamino, N-methyl-N-naphthylamino, the N-methyl-N-1- or -2-phenylethylamino or the N-methyl-N-3-phenylpropylamino radical.

“Aryl-(C₃-C₄)-Alkenylamino” is an aryl radical attached via a (C₃-C₄)-alkenylamino group, for example the 1-, 2- or 3-phenylallylamino radical.

“N-(C₁-C₄)-Alkyl-N-aryl-(C₃-C₄)-alkenylamino” is, for example, the N-methyl-N-1-, -2- or -3-phenylallylamino radical.

“Arylcarbamoyl” is, for example, phenyl- or 1- or 2-naphthylcarbamoyl.

“N-Aryl-N-(C₁-C₄)-alkylcarbamoyl” is, for example, N-methyl-N-phenylcarbamoyl or N-methyl-N-1- or -2-naphthylcarbamoyl.

“Aryl-(C₁-C₈)-dialkylsilyl” is, for example, a phenyl- or naphthyidimethylsilyl group.

“Diaryl-(C₃-C₄)-alkylsilyl” is, for example, a diphenyl, phenylnaphthyl or dinaphthylmethylsilyl group.

“Triarylsilyl” is, for example, a triphenyl, diphenylnaphthyl or trinaphthylsilyl group.

The term that “in the substituents mentioned above which may be present on the radicals R⁶ to R²⁶, hydrogen atoms attached to carbon may be substituted by up to three halogen atoms, in the case of fluorine also up to the maximum number” is to be understood as meaning, for example, the following radicals:

haloalkyl, such as the 1- or 2-fluoroethyl, the trifluoromethyl, the 2,2,2-trifluoroethyl, the chloromethyl, the fluoromethyl, the difluoromethyl or the 1,1,2,2-tetrafluoroethyl group;

haloalkenyl, such as the 1-, 2- or 3-fluoroallyl group or the 1,1-difluoropropen-3-yl group;

haloalkoxy, such as trifluoromethoxy or 2,2,2-trifluoroethoxy;

haloalkylthio, such as trifluoromethylthio;

haloalkylsulfinyl, such as trifluoromethylsulfinyl;

haloalkylsulfonyl, such as trifluoromethylsulfonyl;

halocyclopropyl, such as 1,1-difluorocyclopropyl;

haloalkanoyl, such as trifluoroacetyl;

haloalkylamino, such as 2,2,2-trifluorethylamino.

Depending on the nature of the substituents defined above, the compounds of the formula (I) have acidic or basic properties and are capable of forming salts. If, for example, the compounds of the formula (I) carry groups such as hydroxyl, carboxyl or other groups which induce acidic properties, these compounds can be reacted with bases to form salts. Suitable bases are, for example, hydroxides, carbonates and bicarbonates of the alkali metals and alkaline earth metals, in particular those of sodium, potassium, magnesium and calcium, furthermore ammonia, primary, secondary and tertiary amines with (C₁-C₄) alkyl radicals and mono-, di- and trialkanolamines of (C₁-C₄)-alkanols. If the compounds of the formula (I) carry amino, alkylamino or other groups which induce basic properties, these compounds can be reacted with acids to form salts. Suitable acids are, for example, mineral acids, such as hydrochloric, sulfuric and phosphoric acid, organic acids, such as acetic acid or oxalic acid, and acidic salts, such as NaHSO₄ and KHSO₄. The salts obtainable in this manner likewise have insecticidal, acaricidal and miticidal properties.

The compounds of the formula (I) can have one or more asymmetrically substituted carbon atoms or stereoisomers on double bonds. Therefore, it is possible for enantiomers or diastereomers to be present. The invention embraces both the pure isomers and their mixtures. The mixtures of diastereomers can be separated into the isomers by customary methods, for example by selective crystallization from suitable solvents or by chromatography. Racemates can be separated into the enantiomers by customary methods.

The preparation of the compounds according to the invention is carried out by methods known per se from the literature, as described in standard works on organic synthesis, for example Houben-Weyl, Methoden der Organischen Chemie [Methods of Organic Chemistry], Georg-Thieme-Verlag, Stuttgart.

The preparation is carried out under reaction conditions which are known and suitable for the reactions mentioned. It is also possible to use variants which are known per se but not mentioned here in detail.

If desired, the starting materials can also be formed in situ, i.e. they are not isolated from the reaction mixture but immediately reacted further to give the compounds of the formula (I).

The present invention also relates to processes for preparing compounds of the formula (I).

For preparing compounds of the formula (I) in which R¹, R², R³, R⁴, R⁵, A and n have the meanings given for formula (I) and Y is oxygen, for example, a carboxylic acid of the formula (II) 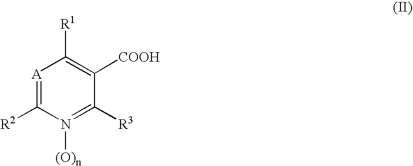
in which A, R¹, R², R³ and n have the meanings given above for formula (I) is, in the form of an activated derivative of this acid, in the presence of a base, reacted with a compound of the formula (III), in which R⁴ and R⁵ have the meanings given for formula (I)
HNR⁴R⁵ (III)
and the radicals R⁴ and R⁵ are then, if required, derivatized further.

Collections of compounds of the formula (I) which can be synthesized by the abovementioned scheme may also be prepared in a parallel manner, and this may be effected manually or in a semiautomated or fully automated manner. In this case, it is possible, for example, to automate the procedure of the reaction, work-up or purification of the products or of the intermediates. In total, this is to be understood as meaning a procedure as is described, for example, by S. H. DeWitt in “Annual Reports in Combinatorial Chemistry and Molecular Diversity: Automated Synthesis”, Volume 1, Verlag Escom 1997, pages 69 to 77.

A series of commercially available apparatuses as are offered by, for example, Stem Corporation, Woodrolfe Road, Tollesbury, Essex, CM9 8SE, England or H+ P Labortechnik GmbH, Bruckmannring 28, 85764 Oberschleiβheim, Germany or by Radleys, Shirehill, Saffron Walden, Essex, England may be used for the parallel procedure of the reaction and work-up. For the parallel purification of compounds of the formula (I), or of intermediates obtained during the preparation, use may be made, inter alia, of chromatography apparatuses, for example those from ISCO, Inc., 4700 Superior Street, Lincoln, Nebr. 68504, USA.

The apparatuses mentioned lead to a modular procedure in which the individual process steps are automated, but manual operations must be performed between the process steps. This can be prevented by employing semi-integrated or fully integrated automation systems where the automation modules in question are operated by, for example, robots. Such automation systems can be obtained, for example, from Zymark Corporation, Zymark Center, Hopkinton, Mass. 01748, USA.

In addition to the methods described here, compounds of the formula (I) may be prepared in part or fully by solid-phase-supported methods. For this purpose, individual intermediate steps or all intermediate steps of the synthesis or of a synthesis adapted to suit the procedure in question are bound to a synthetic resin. Solid-phase-supported synthesis methods are described extensively in the specialist literature, for example Barry A. Bunin in “The Combinatorial Index”, Academic Press, 1998.

The use of solid-phase-supported synthesis methods permits a series of protocols which are known from the literature and which, in turn, can be performed manually or in an automated manner. For example, the “tea-bag method” (Houghten, U.S. Pat. No. 4,631,211; Houghten et al., Proc. Natl. Acad. Sci, 1985, 82, 5131-5135), in which products from IRORI, 11149 North Torrey Pines Road, La Jolla, Calif. 92037, USA are employed, may be semiautomated. The automation of solid-phase-supported parallel syntheses is performed successfully, for example, by apparatuses from Argonaut Technologies, Inc., 887 Industrial Road, San Carlos, Calif. 94070, USA or MultiSynTech GmbH, Wullener Feld 4, 58454 Witten, Germany.

The preparation by the processes described herein yields compounds of the formula (I) in the form of substance collections which are termed libraries.

The present invention also relates to libraries which comprise at least two compounds of the formula (I).

The compounds of the formula (I) are suitable for controlling animal pests, in particular insects, arachnids, helminths and molluscs, preferably for controlling insects and arachnids which are encountered in agriculture, in livestock breeding, in forests, in the protection of stored goods and materials, and in the hygiene sector, and have good plant tolerance and favorable toxicity to warm-blooded species. They are active against normally sensitive and resistant species and against all or individual developmental stages. The abovementioned pests include:

From the order of the Acarina, for example, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Eotetranychus spp., Oligonychus spp., Eutetranychus spp.

From the order of the Isopoda, for example, Oniscus aselus, Armadium vulgare, Porcellio scaber.

From the order of the Diplopoda, for example, Blaniulus guttulatus.

From the order of the Chilopoda, for example, Geophilus carpophagus, Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Blatta orientalis, Periplaneta americana, Leucophaea maderae, Blattella germanica, Acheta domesticus, Gryllotalpa spp., Locusta migratoria migratorioides, Melanoplus differentialis, Schistocerca gregaria.

From the order of the Isoptera, for example, Reticulitermes spp.

From the order of the Anoplura, for example, Phylloera vastatrix, Pemphigus spp., Pediculus humanus corporis, Haematopinus spp., Linognathus spp.

From the order of the Mallophaga, for example, Trichodectes pp., Damalinea spp.

From the order of the Thysanoptera, for example, Hercinothrips femoralis, Thrips tabaci.

From the order of the Heteroptera, for example, Eurygaster spp., Dysdercus intermedius, Piesma quadrata, Cimex lectularius, Rhodnius prolixus, Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Doralis fabae, Doralis pomi, Eriosoma lanigerum, Hyalopterus arundinis, Macrosiphum avenae, Myzus spp., Phorodon humuli, Rhopalosiphum padi, Empoasca spp., Euscelus bilobatus, Nephotettix cincticeps, Lecanium corni, Saissetia oleae, Laodelphax striatellus, Nilaparvata lugens, Aonidiella aurantii, Aspidiotus hederae, Pseudococcus spp., Psylla spp.

From the order of the Lepidoptera, for example, Pectinophora gossypiella, Bupalus piniarius, Cheimatobia brumata, Lithocolletis blancardella, Hyponomeuta padella, Plutella maculipennis, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Laphygma exigua, Mamestra brassicae, Panolis flammea, Prodenia litura, Spodoptera spp., Trichoplusia ni, Carpocapsa pomonella, Pieris spp., Chilo spp., Pyrausta nubilalis, Ephestia kuehniella, Galleria mellonella, Cacoecia podana, Capua reticulana, Choristoneura fumiferana, Clysia ambiguella, Homona magnanima, Tortrix viridana.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylloides chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrynchus assimilis, Hypera postica, Dermestes spp., Trogoderma, Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica.

From the order of the Hymenoptera, for example, Diprion spp., Hoplocampa spp., Lasius spp., Monomorium pharaonis, Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hypobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa.

From the order of the Siphonaptera, for example, Xenopsylla cheopsis, Ceratophyllus spp.

From the order of the Arachnida, for example, Scorpio maurus, Latrodectus mactans.

From the class of the helminths, for example, Haemonchus, Trichostrongulus, Ostertagia, Cooperia, Chabertia, Strongyloides, Oesophagostomum, Hyostrongulus, Ancylostoma, Ascaris and Heterakis and also Fasciola.

From the class of the Gastropoda, for example, Deroceras spp., Arion spp., Lymnaea spp., Galba spp., Succinea spp., Biomphalaria spp., Bulinus spp., Oncomelania spp.

From the class of the Bivalva, for example, Dreissena spp.

It is furthermore possible to control Protozoa, such as Eimeria.

The plant-parasitic nematodes which can be controlled in accordance with the invention include, for example, the root-parasitic soil-dwelling nematodes such as, for example, those of the genera Meloidogyne (root knot nematodes, such as Meloidogyne incognita, Meloidogyne hapla and Meloidogyne javanica), Heterodera and Globodera (cyst-forming nematodes, such as Globodera rostochiensis, Globodera pallida, Heterodera trifolii) and of the genera Radopholus, such as Radopholus similis, Pratylenchus such as Pratylenchus neglectus, Pratylenchus penetrans and Pratylenchus curvitatus; Tylenchulus such as Tylenchulus semipenetrans, Tylenchorhynchus, such as Tylenchorhynchus dubius and Tylenchorhynchus claytoni, Rotylenchus such as Rotylenchus robustus, Heliocotylenchus such as Haliocotylenchus multicinctus, Belonoaimus such as Belonoaimus longicaudatus, Longidorus such as Longidorus elongatus, Trichodorus such as Trichodorus primitivus and Xiphinema such as Xiphinema index.

Nematode genera which can furthermore be controlled using the compounds according to the invention are Ditylenchus (stem parasites, such as Ditylenchus dipsaci and Ditylenchus destructor), Aphelenchoides (foliar nematodes, such as Aphelenchoides ritzemabosi) and Anguina (flower and leaf-gall nematodes, such as Anguina tritici).

The compounds according to the invention are preferably suitable for controlling sucking insects, such as aphids (for example Aphis fabae, Aphis pomi, Aphis spiraecola, Aphis gossypii, Aphis nasturtii, Dysaphis plantaginea, Eriosoma spp., Rhopalosiphum padi, Acyrthosiphon pisum, Pemphigus bursarius, Myzus persicae, Myzus nicotianae, Myzus euphorbiae, Phylloxera spp., Toxoptera spp, Brevicoryne brassicae, Macrosiphum avenae, Macrosiphum euphorbiae, Nasonovia ribisnigri, Sitobion avenae, Brachycaudus helychrysii or Phorodon humuli), cicadas (Idioscopus clypealis, Scaphoides titanus, Empoasca onuki, Empoasca vitis, Empoasca devastans, Empoasca libyca, Empoasca biguttula, Empoasca facialis, or Erythroneura spp), Thrips (Hercinothrips femoralis, Scirtothrips aurantii, Scirtothrips dorsalis, Frankliniella schultzei, Frankliniella fusca, Frankliniella occidentalis, Frankliniella tritici, Kakothrips spp., Thrips oryzae, Thrips palmi, Thrips tabaci) or white flies (Aleurodes brassicae, Bemisia tabaci, Trialeurodes vaporariorum, Aleurodes proletella).

The invention also relates to compositions, for example crop protection compositions, preferably insecticidal, acaricidal, ixodicidal, nematicidal, molluscicidal or fungicidal, especially preferably insecticidal and acaricidal, compositions which comprise one or more compounds of the formula (I) in addition to suitable formulation auxiliaries.

To prepare the compositions according to the invention, the active compound and the other additives are combined and brought into a suitable use form.

In general, the compositions according to the invention comprise 1 to 95% by weight of the active compounds of the formula (I). They can be formulated in various ways, depending on the biological and/or chemical-physical parameters which prevail. The following are examples of possible formulations:

Wettable powders (WP), emulsifiable concentrates (EC), aqueous solutions (SL), emulsions, sprayable solutions, oil- or water-based dispersions (SC), suspo-emulsions (SE), dusts (DP), seed-dressing products, granules in the form of microgranules, spray granules, coated granules and adsorption granules, water-dispersible granules (WG), ULV formulations, microcapsules, waxes or baits.

These individual types of formulations are known in principle and are described, for example, in: Winnacker-Kuchler, “Chemische Technologie” [Chemical Technology], Volume 7, C. HanserVerlag Munich, 4th Edition 1986; van Falkenberg, “Pesticides Formulations”, Marcel Dekker N.Y., 2nd Ed. 1972-73; K. Martens, “Spray Drying Handbook”, 3rd Ed. 1979, G. Goodwin Ltd. London.

The necessary formulation auxiliaries, i.e. carrier materials and/or surface-active compounds such as inert materials, surfactants, solvents and other additives, are also known and described, for example, in: Watkins, “Handbook of Insecticide Dust Diluents and Carriers”, 2nd Ed., Darland Books, Caldwell N.J.; H. v. Olphen, “Introduction to Clay Colloid Chemistry”, 2nd Ed., J. Wiley & Sons, N.Y.; Marsden, “Solvents Guide”, 2nd Ed., Interscience, N.Y. 1950; McCutcheon's, “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schonfeldt, “Grenzflatchenaktive Athylenoxidaddukte” [Surface-active ethylene oxide adducts], Wiss. Verlagsgesell., Stuttgart 1967; Winnacker-Kuchler, “Chemische Technologie”, Volume 7, C. HanserVerlag Munich, 4th Edition 1986.

Based on these formulations, it is also possible to prepare combinations with other pesticidally active materials, fertilizers and/or growth regulators, for example in the form of a ready-mix formulation or a tank mix. Wettable powders are preparations which are uniformly dispersible in water which, besides the active compound, also comprise wetters, for example polyoxyethylated alkylphenols, polyoxyethylated fatty alcohols, alkylsulfonates or alkylphenolsulfonates and dispersants, for example sodium lignosulfonate or sodium 2,2′-dinaphthylmethane-6,6′-disulfonate, in addition to a diluent or inert material.

Emulsifiable concentrates are prepared by dissolving the active compound in an organic solvent, for example butanol, cyclohexanone, dimethylformamide, xylene or else higher-boiling aromatics or hydrocarbons, with addition of one or more emulsifiers. As emulsifiers, the following can be used, for example: calcium alkylarylsulfonates such as calcium dodecylbenzenesulfonate, or nonionic emulsifiers such as fatty acid polyglycol esters, alkylaryl polyglycol ethers, fatty alcohol polyglycol ethers, propylene oxide/ethylene oxide condensates, alkyl polyethers, sorbitan fatty acid esters, polyoxyethylene sorbitan fatty acid esters or polyoxyethylene sorbitol esters.

Dusts are obtained by grinding the active compound with finely divided solid materials, for example talc or natural clays, such as kaolin, bentonite, pyrophyllite or diatomaceous earth. Granules can be prepared either by atomizing the active compound onto adsorptive, granulated inert material or by applying active compound concentrates onto the surface of carrier materials such as sand or kaolinites, or of granulated inert material, by means of adhesives, for example polyvinyl alcohol or sodium polyacrylate, or else mineral oils. Suitable active compounds can also be granulated in the manner which is customary for the preparation of fertilizer granules, if desired as a mixture with fertilizers.

The active compound concentration in wettable powders is usually approximately 10 to 90% by weight, the remainder to 100% by weight is composed of customary formulation constituents. In the case of emulsifiable concentrates, the active compound concentration may be approximately 5 to 80% by weight. Formulations in the form of dusts usually comprise 5 to 20% by weight of active compound, sprayable solutions approximately 2 to 20% by weight. In the case of granules, the active compound content depends partly on whether the active compound is in liquid or solid form and on which granulation auxiliaries, fillers and the like are being used.

Besides this, the abovementioned active compound formulations comprise, if appropriate, the tackifiers, wetters, dispersants, emulsifiers, penetrants, solvents, fillers or carriers which are conventional in each case.

For use, the concentrates, which are present in commercially available form, are, if desired, diluted in the customary manner, for example in the case of wettable powders, emulsifiable concentrates, dispersions and in some cases also microgranules, using water. Preparations in the form of dusts and granules and sprayable solutions are usually not diluted any further with other inert substances prior to use.

The application rate required varies with the external conditions such as, inter alia, temperature and humidity. It may vary within wide limits, for example between 0.0005 and 10.0 kg/ha or more of active compound, but it is preferably between 0.001 and 5 kg/ha of active compound.

The active compounds according to the invention, in their commercially available formulations and in the use forms prepared from these formulations, may be present in mixtures with other active compounds such as insecticides, attractants, sterilants, acaricides, nematicides, fungicides, growth regulatory substances or herbicides.

The pesticides include, for example, phosphoric esters, carbamates, carboxylic esters, formamidines, tin compounds and materials produced by microorganisms.

Preferred components in mixtures are:

1. from the group of the phosphorus compounds acephate, azamethiphos, azinphos-ethyl, azinphos-methyl, bromophos, bromophos-ethyl, cadusafos, chlorethoxyphos, chlorfenvinphos, chlormephos, chlorpyrifos, chlorpyrifos-methyl, demeton, demeton-S-methyl, demeton-S-methyl sulfone, dialifos, diazinon, dichlorvos, dicrotophos, dimethoate, disulfoton, EPN, ethion, ethoprophos, etrimfos, famphur, fenamiphos, fenitriothion, fensulfothion, fenthion, flupyrazofos, fonofos, formothion, fosthiazate, heptenophos, isazophos, isothioate, isoxathion, malathion, methacrifos, methamidophos, methidathion, salithion, mevinphos, monocrotophos, naled, omethoate, oxydemeton-methyl, parathion, parathion-methyl, phenthoate, phorate, phosalone, phosfolan, phosphocarb (BAS-301), phosmet, phosphamidon, phoxim, pirimiphos, pirimiphos-ethyl, pirimiphosmethyl, profenofos, propaphos, proetamphos, prothiofos, pyraclofos, pyridapenthion, quinalphos, sulprofos, temephos, terbufos, tebupirimfos, tetrachlorvinphos, thiometon, triazophos, trichlorphon, vamidothion;

2. from the group of the carbamates alanycarb (OK-135), aldicarb, 2-sec-butylphenyl methylcarbamate (BPMC), carbaryl, carbofuran, carbosulfan, cloethocarb, benfuracarb, ethiofencarb, furathiocarb, HCN-801, isoprocarb, methomyl, 5-methyl-m-cumenylbutyryl (methyl)carbamate, oxamyl, pirimicarb, propoxur, thiodicarb, thiofanox, 1-methylthio(ethylideneamino)-N-methyl-N-(morpholinothio)carbamate (UC 51717), triazamate;

3. from the group of the carboxylic esters acrinathrin, allethrin, alphametrin, 5-benzyl-3-furylmethyl (E)-(1R)-cis-2,2-dimethyl-3-(2-oxothiolan-3-ylidenemethyl)cyclopropanecarboxylate, beta-cyfluthrin, beta-cypermethrin, bioallethrin, bioallethrin ((S)-cyclopentylisomer), bioresmethrin, bifenthrin, (RS)-1-cyano-1-(6-phenoxy-2-pyridyl)methyl (1RS)-trans-3-(4-tert-butylphenyl)-2,2-dimethylcyclopropanecarboxylate (NCI 85193), cycloprothrin, cyfluthrin, cyhalothrin, cythithrin, cypermethrin, cyphenothrin, deltamethrin, empenthrin, esfenvalerate, fenfluthrin, fenpropathrin, fenvalerate, flucythrinate, flumethrin, fluvalinate (D isomer), gamma-cyhalothrin, imiprothrin (S41311), lambda-cyhalothrin, permethrin, phenothrin ((R) isomer), prallethrin, pyrethrins (natural products), resmethrin, tebupirimfos, tefluthrin, tetramethrin, theta-cypermethrin (TD-2344), tralomethrin, transfluthrin, zeta-cypermethrin (F-56701);

4. from the group of the amidines amitraz, chlordimeform;

5. from the group of the tin compounds cyhexatin, fenbutatin oxide;

6. others Abamectin, ABG-9008, acequinocyl, azadirachtin, acetamiprid, Anagrapha falcitera, AKD-1022, AKD-3088, AL-9811, ANS-118, Bacillus thuringiensis, Beauveria bassianea, bensultap, bifenazate, binapacryl, bistrifluron, BJL-932, bromopropylate, BTG-504, BTG-505, BTG-514, BTG-522, buprofezin, camphechlor, cartap, chlorobenzilate, chlorfenapyr, chlorfluazuron, 2-(4-chlorophenyl)-4,5-diphenylthiophene (UBI-T 930), chlorfentezine, chloproxyfen, clothianidine, chromafenozide, A-184699, 2-naphthylmethyl cyclopropancarboxylate (Rol 2-0470), CM-002×, DBI-3204, cyromazin, diacloden (thiamethoxam), diafenthiuron, ethyl N-(3,5-dichloro-4-(1,1,2,3,3,3-hexafluoro-1-propyloxy)phenyl)carbamoyl)-2-chlorobenzocarboximidate, DDT, dicofol, diflubenzuron, N-(2,3-dihydro-3-methyl-1,3-thiazol-2-ylidene)-2,4-xylidine, dinobuton, dinocap, dinotefuran, diofenolan, emamectin-benzoate, endosulfan, ethiprole (sulfethiprole), ethofenprox, etoxazole, fenazaquin, fenoxycarb, fipronil, fluazuron, flonicamid, flumite (flufenzine, SZI-121), 2-fluoro-5-(4-(4-ethoxyphenyl)-4-methyl-1-pentyl)diphenylether (MTI 800), granulosis and nuclear polyhedrosis viruses, fenpyroximate, fenthiocarb, fluacrypyrim, flubenzimine, flucycloxuron, flufenerim, flufenoxuron, flufenprox, fluproxyfen, flufenzine, FMC-F6028, gamma-HCH, halofenozide, halofenprox, hexaflumuron (DE_{—}473), hexythiazox, HO1-9004, hydramethylnon (AC 217300), lufenuron, imidacloprid, indoxacarb, kanemite (AKD-2023), M-020, ivermectin, methoxyfenozide, milbemectin, MKI-245, NC-196, NC-510, neemgard, nidinotefuran, nitenpyram, 2-nitromethyl4,5-dihydro-6H-thiazine (DS 52618), 2-nitromethyl-3,4-dihydrothiazole (SD 35651), 2-nitromethylene-1,2-thiazinan-3-ylcarbamaldehyde (WL 108477), novaluron, noviflumuron, pyriproxyfen (S-71639), NC-196, NC-1111, NNI-9768, OK-9701, OK-9601, OK-9602, propargite, pymethrozine, pyridaben, pyridaryl, pyrimidifen, pyriproxifen, RYI-210, S-1283, S-1833, SB7242, SI-8601, silafluofen, silomadine (CG-177), spinosad, spirodiclofen, spiromesifen, SU-9118, tebufenozide, tebufenpyrad, teflubenzuron, tefluthrin, tetradifon, tetrasul, thiacloprid, thiametoxam, thiocyclam, tolfenpyrad, triazamate, triflumuron, verbutin, vertalec (mykotal), YI-5301.

The abovementioned components for combinations are known active compounds, many of which are described in C. D. S. Tomlin (Ed.), The Pesticide Manual, 12th Edition, British Crop Protection Council, Farnham 2000.

The active compound content of the use forms prepared from the commercially available formulations may range from 0.00000001 up to 95% by weight of active compound, preferably between 0.00001 and 1% by weight.

Application is effected in a customary manner adapted to suit the use forms.

Accordingly, the invention also provides the use of compounds of the formula (I) and salts thereof for controlling animal pests, preferably harmful arthropods, such as insects and arachnids, helminths and/or nematodes.

The invention furthermore provides a method for controlling harmful insects, arachnids and/or helminths which comprises applying an effective amount of a compound of the formula (I) or a salt thereof onto the pests or the site of the desired action.

The active compounds according to the invention are also suitable for controlling endoparasites and ectoparasites in the veterinary medicine sector and/or in the field of animal keeping. The active compounds according to the invention are applied here in a known manner, such as by oral administration in the form of, for example, tablets, capsules, drinks or granules, by dermal application in the form of, for example, dipping, spraying, pouring on and spotting on, and dusting, and by parenteral administration in the form of, for example, an injection.

Accordingly, the invention also provides the use of compounds of the formula (I) or of a salt thereof for preparing a medicament for human and/or veterinary medicine, preferably a medicament for veterinary medicine, in particular for the control of ecto- and/or endoparasites.

Accordingly, the compounds of the formula (I) can also be employed advantageously in livestock keeping (for example cattle, sheep, pigs and poultry such as chickens, geese and the like). In a preferred embodiment of the invention, the compounds, if appropriate in suitable formulations, are administered orally to the animals, if appropriate together with the drinking water or feed. Since excretion in the feces is efficient, the development of insects in the animals' feces can be prevented very easily in this manner. The dosages and formulations which are suitable in each case depend, in particular, on the species and the developmental stage of the productive livestock and also on the risk of infestation and can be determined readily and established by customary methods. For example, the compounds can be employed in cattle at dosages of 0.01 to 1 mg/kg of bodyweight.

In addition to the abovementioned application methods, the active compounds of the formula (I) according to the invention have excellent systemic action. Accordingly, the active compounds can also be introduced into the plants via parts of the plant, both below ground and above ground (root, stem, leaf), if the active compounds are applied, in liquid or solid form in the direct vicinity of the plant (for example granules in soil application, application in flooded rice paddies).

Furthermore, the active compounds according to the invention are particularly useful for the treatment of vegetative and generative plant propagation material, such as, for example, of seeds, for example of cereals, vegetables, cotton, rice, sugar beet and other crops and ornamental plants, of bulbs, seedlings and tubers of other crops and ornamental plants which are propagated vegetatively. The treatment can be carried out before sowing or before planting (for example by special seed coating techniques, by dressing in liquid or solid form or as a seed box treatment), during sowing or planting or after sowing or planting by special application techniques (for example furrow treatment). The amount of active compound used can vary within a relatively large range, depending on the application. In general, the application rates are between 1 g and 10 kg of active compound per hectare of soil surface.

The compounds of the formula (I) can also be employed for controlling animal pests in crops of known genetically engineered plants or genetically engineered plants yet to be developed. As a rule, the transgenic plants are distinguished by especially advantageous properties, for example by resistances to particular crop protection agents, resistances to plant diseases or pathogens of plant diseases, such as particular insects or microorganisms such as fungi, bacteria or viruses. Other particular properties concern, for example, the harvested material with regard to quantity, quality, storage properties, composition and specific constituents. Thus, transgenic plants are known where the starch content is increased, or the starch quality is altered, or where the harvested material has a different fatty acid composition.

The use in economically important transgenic crops of useful plants and ornamentals is preferred, for example of cereals such as wheat, barley, rye, oats, millet, rice, cassaya and corn or else crops of sugar beet, cotton, soybean, oilseed rape, potatoes, tomatoes, peas and other types of vegetables.

When used in transgenic crops, in particular those which have resistances to insects, effects are frequently observed, in addition to the effects against harmful organisms to be observed in other crops, which are specific for application in the transgenic crop in question, for example an altered or specifically widened spectrum of pests which can be controlled, or altered application rates which may be employed for application.

The invention therefore also relates to the use of compounds of the formula (I) for controlling harmful organisms in transgenic crop plants.

The use of the compounds according to the invention embraces, in addition to direct application onto the pests, any other application in which compounds of the formula (I) act on the pests. Such indirect applications can, for example, be the use of compounds which, for example in the soil, the plant or the pest, decompose into compounds of the formula (I) and/or are degraded into compounds of the formula (I).

In addition to their lethal effect on pests, the compounds of the formula (I) or their salts also have a pronounced repellent effect.

A repellent for the purpose of the description is a substance or substance mixture which has a warding-off or fending-off effect on other living beings, in particular harmful pests and nuisance pests. The term also encompasses effects such as the antifeeding effect, where the intake of feed is disturbed or prevented (antifeedant effect), suppression of oviposition, or an effect on the development of the population.

The invention therefore also provides the use of compounds of the formula (I) or their salts for achieving the abovementioned effects, in particular in the case of the pests stated in the biological examples.

The invention also provides a method for fending off, or warding off, harmful organisms, where one or more compounds of the formula (I) or their salts are applied to the site from which the harmful organisms are to be fended off or warded off.

In the case of a plant, application may mean, for example, a treatment of the plant, but also of the seed.

As regards the effect on populations, it is interesting to note that effects can also be observed in succession during the development of a population, where summation may take place. In such a case, the individual effect itself may only have an efficacy of markedly less than 100% but in total an efficacy of 100% is still achieved in the end.

Moreover, the compounds of the formula (I) or their salts are distinguished by the fact that the composition is usually applied earlier than in the case of a direct control, if the abovementioned effects are to be exploited. The effect frequently lasts over a long period, so that a duration of action of over 2 months is achieved.

The effects are observed in insects, arachnids and the other abovementioned pests.

The examples below serve to illustrate the invention.

### A. CHEMICAL EXAMPLES

### Example A

### N-[2-(benzylthio)ethyl]-4-trifluoromethylnicotinamide

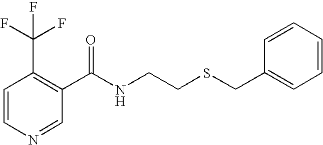

At 0° C., a solution of 1.05 g (5.0 mmol) of 4-trifluoromethylnicotinoyl chloride in a little dichloromethane was added dropwise to a solution of 1.02 g (5.0 mmol) of 2-(benzylthio)ethylamine hydrochloride and 1.01 g (10.00 mmol) of triethylamine in 50 ml of dichloromethane. The mixture was stirred at room temperature for two more hours, saturated sodium chloride solution was added and the phases were separated. The aqueous phase was extracted two more times with dichloromethane. The combined organic phases were dried and concentrated. This gave 1.26 g (74.4% of theory) of product as a colorless solid of melting point 86-88° C.

### Example B

### N-[2-(benzylsulfinyl)ethyl]-4-trifluoromethylnicotinamide

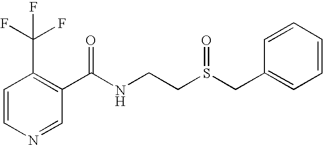

At 0° C., a solution of 1.03 g (4.2 mmol) of 70% strength 3-chloroperoxybenzoic acid in 25 ml of dichloromethane was added dropwise to a solution of 1.43 g (4.2 mmol) of N-[2-(benzylthio)ethyl]-4-trifluoromethylnicotinamide (Example A) in 50 ml of dichloromethane. The mixture was stirred at room temperature for 6 hours and then extracted with sodium carbonate solution, and the organic phase was dried. Concentration gave a colorless oil which, for purification, was chromatographed on silica gel (ethyl acetate). This gave 1.22 g (81.5% of theory) of product in the form of colorless crystals.

M.p.: 130-132° C.

### Example C

### N-[2-(benzylsulfonyl)ethyl]4-trifluoromethylnicotinamide

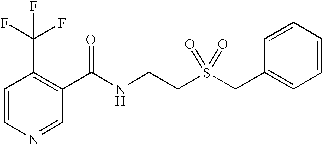

At 0° C., a solution of 2.17 g (8.8 mmol) of 70% strength 3-chloroperoxybenzoic acid in 25 ml of dichloromethane was added dropwise to a solution of 1.43 g (4.2 mmol) of N-[2-(benzylthio)ethyl]-4-trifluormethylnicotinamide (Example A) in 50 ml of dichloromethane. The mixture was stirred at room temperature for 6 hours and then extracted with sodium carbonate solution, and the organic phase was dried. Concentration gave a colorless oil which, for purification, was chromatographed on silica gel (heptane/ethyl acetate 9:1). This gave 0.94 g (60.4% of theory) of product in the form of colorless crystals.

M.p.: 144-146° C.

### Example D

### N-[3-(2,2,2-trifluoroethylthio)propyl]-4-trifluoromethylnicotamide

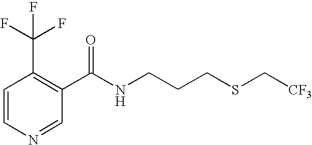

At room temperature, a solution of 4.66 g (15 mmol) of N-(3-bromopropyl)-4-trifluoromethylnicotinamide in 20 ml of methanol was added dropwise to a mixture of 2.97 g (16.5 mmol) of a 30% strength solution of sodium methoxide in methanol and 1.74 g (15 mmol) of 2,2,2-trifluoroethylmercaptan in 40 ml of methanol. The mixture was stirred at room temperature for 4 hours and at 50° C. for 2 hours. The solvent was removed under reduced pressure and the residue was then taken up in water/dichloromethane. For purification, the mixture was chromatographed on silica gel (ethyl acetate/heptane 7:3). This gave 4.7 g (90.1% of theory) of a colorless oil which crystallized on standing.

M.p. 85-86° C.

Preparation of the starting material N-(3-bromopropyl)-4-trifluoromethylnicotinamide

At 0° C., a solution of 24.39 g (240 mmol) of triethylamine in 50 ml of dichloromethane was added dropwise to a mixture of 26.05 g (119 mmol) of 3-bromo-1-propylamine hydrobromide and 24.94 g (119 mmol) of 4-trifluoromethylnicotinoyl chloride. The mixture was stirred at room temperature for 4 hours and then concentrated, and the residue was taken up in water/dichloromethane. After phase separation, the aqueous phase was extracted two more times with dichloromethane, and the combined organic phases were dried and concentrated. This gave 32.71 g of product (88.4% of theory) as a colorless solid which was reacted further without further purification.

M.p. 61-63° C.

### Example E

### N-[2-(3-methylbut-2-enylthio)ethyl]-4-trifluoromethylnicotinamide

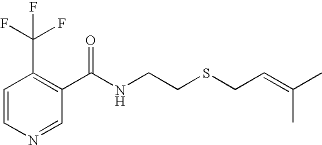

0.89 g (6 mmol) of 1-bromo-3-methylbut-2-ene was added to a suspension of 1.49 g (4 mmol) of N-(4-trifluoromethylnicotinoyl)-2-ethylaminoisothiuronium hydrobromide (preparation: Example F) in 15 ml of isopropanol/ethanol (4:1), and, at room temperature, a solution of 0.68 g (17 mmol) of caustic soda in 2 ml of water was added dropwise. This gave a clear solution which was stirred at room temperature for 4 hours and at 50° C. for 2 hours. After cooling to room temperature, the mixture was neutralized by addition of dry ice and concentrated. The residue was taken up in water/dichloromethane and the organic phase was dried and concentrated. For purification, the mixture was chromatographed on silica gel (ethyl acetate/heptane 9:1). This gave 1.05 g (82.5% of theory) of product as a colorless solid.

M.p.: 76-77° C.

### Example F

### N-(4-Trifluoromethylnicotinoyl)-2-ethylaminoisothiuronium hydrobromide

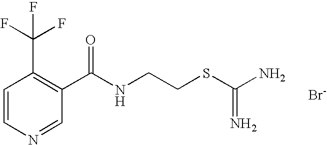

39.49 g (133 mmol) of N-(4-trifluoromethylnicotinoyl)-2-bromoethylamide (prepared analogously to Example D from 4-trifluoromethylnicotinoyl chloride and 2-bromoethylamine hydrochloride) and 10.12 g (133 mmol) of thiourea were heated under reflux in 130 ml of ethanol for 6 hours. After cooling, the crystalline product was filtered off with suction.

Yield 37.66 g (76.1% of theory, m.p.: 227° C.

### Example G

### N-[3-(benzylthio)propyl]4-trifluoromethylnicotinamide

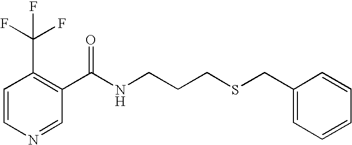

Prepared analogously to Example D from N-(3-bromopropyl)-4-trifluormethyl-nicotinamide and benzylmercaptan sodium salt.

Yield: 82.5% of theory

M.p. 88-89° C.

### Example H

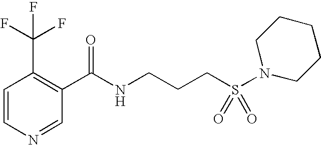

### N-(4-Trifluoromethylnicotinoyl)-3-aminopropiosulfonic acid piperidide

5.32 g (15 mmol) of N-[3-(benzylthio)propyl]4-trifluoromethylnicotinamide (Example G) were initially charged in a mixture of 3.5 ml of water and 25 ml of glacial acetic acid, and chlorine gas was introduced with ice cooling. After 20 minutes, the reaction had ended. The mixture was diluted with water and extracted repeatedly with dichloromethane, and the combined organic phases were extracted twice with aqueous bicarbonate solution, dried and concentrated. The crude acid chloride was purified by chromatography on silica gel (ethyl acetate). This gave 1.2 g (24.2% of theory) of acid chloride as a resin which slowly crystallized.

To prepare the sulfonamide, a mixture of 0.09 g (1 mmol) of piperidine and 0.10 g (1 mmol) of triethylamine was initially charged in 25 ml of dichloromethane. At room temperature, a solution of 0.33 g (1 mmol) of the sulfonyl chloride described above in a little dichloromethane was added dropwise. The mixture was allowed to stand overnight, concentrated and then worked up with water/dichloromethane. This gave 0.14 g (36.9% of theory) of product as a colorless solid.

M.p.: 130° C.

### Example I

### N-(4-Trifluoromethylnicotinoyl)-3-aminopropionaldehyde O-methyl oxime

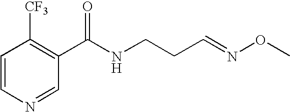 1.23 g (5 mmol) of N-(4-trifluoromethylnicotinoyl)-3-aminopropionaldehyde, 0.42 g (5 mmol) of O-methyl hydroxylamine hydrochloride and 0.41 g (5 mmol) of sodium acetate in 30 ml of methanol were heated under reflux for 6 hours. After cooling, the mixture was concentrated, the residue was taken up in dichloromethane/sodium carbonate solution and the organic phase was dried and concentrated. For purification, the residue was chromatographed on silica gel (ethyl acetate/methanol 9:1). This gave 1.12 g (79.9% of theory) of product (isomer mixture syn/anti about 1:1) as a colorless oil which slowly solidified.

M.p. 80-82° C.

### Preparation of the starting material N-(4-trifluoromethylnicotinoyl)-3-aminopropionaldehyde

Analogously to Example A, initially N-(4-trifluoromethylnicotinoyl)-3-aminopropionaldehyde diethyl acetal was prepared from 1-aminopropionaldehyde diethyl acetal and 4-trifluormethylnicotinoyl chloride in the presence of triethylamine (colorless solid, m.p. 59-60° C., yield 91.8% of theory).

The acetal was hydrolyzed by stirring with formic acid to which some water had been added (1 hour, 30-40° C.). For work-up, the formic acid was removed under reduced pressure, the residue was taken up in dichloromethane/sodium bicarbonate solution and the organic phase was dried and concentrated. This gave the product as a colorless oil (73.1% of theory) which was reacted further without further purification.

### Example J

### N-tert-Butoxycarbonyl-N′-(4-trifluoromethylnicotinoyl)ethylenediamine

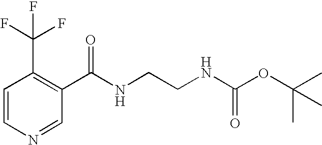

At room temperature, 1.3 g (6.2 mmol) of 4-trifluoromethylnicotinoyl chloride were added dropwise to a solution of 1.0 g (6.2 mmol) of BOC-ethylenediamine and 1.0 ml (7.4 mmol) of triethylamine in 25 ml of dichloromethane. The mixture was allowed to stand overnight and then concentrated, and the residue was triturated with diethyl ether and filtered off with suction. The filtrate was concentrated, and the oily residue was triturated with heptane. Filtration with suction gave 1.95 g (94.4% of theory) of product in the form of colorless crystals.

M.p.: 114-115° C.

### Example K

### N-Methylthiocarbonyl-N′-(4-trifluoromethylnicotinoyl)ethylenediamine

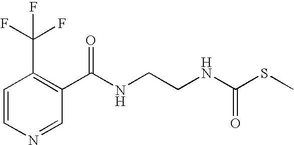

15.45 g (0.05 mol) of N-tert-butoxycarbonyl-N′-(4-trifluoromethylnicotinoyl)-ethylenediamine (Example J) were dissolved in 100 ml of dioxane, and 50 ml of a solution of gaseous hydrogen chloride in dioxane were added. The mixture was stirred at room temperature for 4 hours and under reflux for one hour. The solid that remained was triturated with toluene and again concentrated to dryness. This gave, in quantitative yield, N-(4-trifluoromethylnicotinoyl)ethylenediamine hydrochloride as a colorless solid which was reacted further without further purification.

0.75 g (2.8 mmol) of N-(4-trifluoromethylnicotinoyl)ethylenediamine and 0.97 ml (7.0 mmol) of triethylamine were initially charged in 50 ml of dichloromethane. At room temperature, 0.31 g (2.8 mmol) of methyl chlorothioformate was added dropwise. The mixture was stirred at room temperature for 4 hours and then extracted with saturated sodium chloride solution, and the organic phase was dried and concentrated. The crude product was chromatographed on silica gel (ethyl acetate). This gave 0.56 g (65.5% of theory) of product in the form of colorless crystals.

M.p.: 156° C.

The compounds of the formula (I) listed in the tables below are prepared in an analogous manner.

TABLE 1

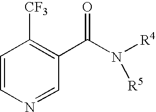

| Ex. No. | NR⁴R⁵ | m.p.[° C.] |
|---|---|---|
| 1 | 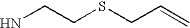 | |
| 2 | 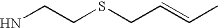 | 45-48 |
| 3 | 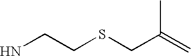 | 56-57 |
| 4 | 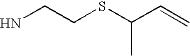 | |
| 5 | 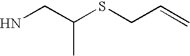 | |
| 6 | 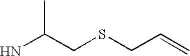 | |
| 7 | 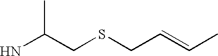 | |
| 8 | 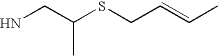 | |
| 9 | 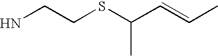 | |
| 10 | 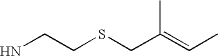 | |
| 11 | 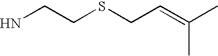 | 76 |
| 12 | 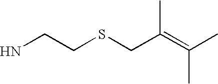 | |
| 13 | 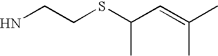 | |
| 14 | 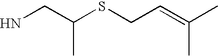 | |
| 15 | 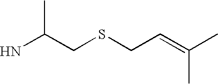 | |
| 16 | 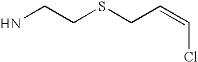 | 45-48 |
| 17 | 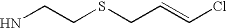 | |
| 18 | 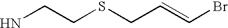 | 47-50 |
| 19 | 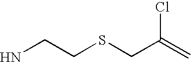 | 59 |
| 20 | 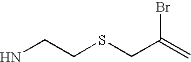 62-64 | |
| 21 | 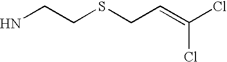 | 65-68 |
| 22 | 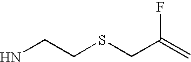 | |
| 23 | 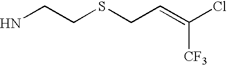 | |
| 24 | 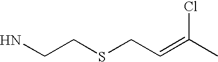 | 46-49 |
| 25 | 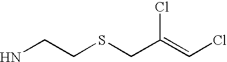 | |
| 26 | 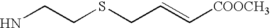 | |
| 27 | 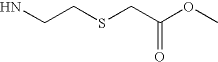 | |
| 28 | 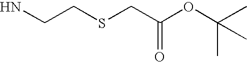 | |
| 28a | 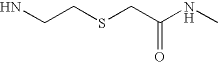 | 130-131 |
| 29 | 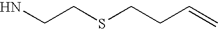 | oil, NMR (CDCl3): 5.92(m, 1H, olef. H); 5.10 (m, 2H, olef. H) |
| 30 | 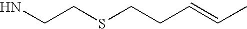 | oil, NMR (CDCl3): 5.62(m, 1H, olef. H); 5.44 (m, 1H, olef. H); 1.72(d, 3H, CH₃) |
| 31 | 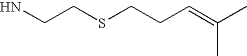 | oil, NMR (CDCl3): 5.22(m, 1H, olef. H); 1.74 (s, 3H, CH₃); 1.67(s, 3H, CH₃) |
| 32 | 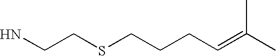 | |
| 33 | 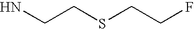 | oil, NMR (CDCl3): 4.68 tr, 4.54 tr, 2H, CH₂F) |
| 34 | 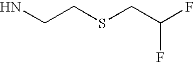 | 74-75 |
| 35 | 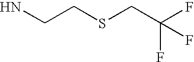 | 83 |
| 36 | 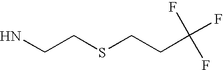 | 92-94 |
| 37 | 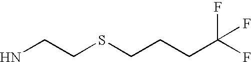 | |
| 38 | 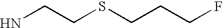 | |
| 39 | 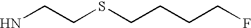 | |
| 40 | 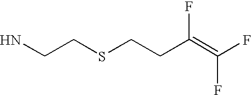 | |
| 41 | 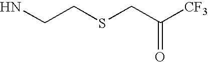 | |
| 42 | 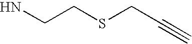 | |
| 43 | 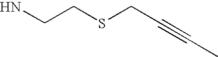 | |
| 44 | 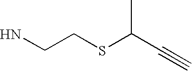 | |
| 45 | 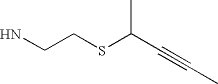 | |
| 46 | 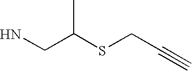 | |
| 47 | 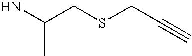 | |
| 48 | 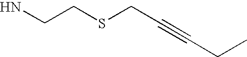 | |
| 49 | 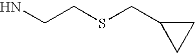 | |
| 50 | 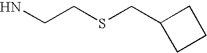 | oil, NMR (CDCl3): 2.50 (sept, 1H, methine-H); 2.12, 1.86, 1.68(3m, 3 CH₂, cyclobutyl-H) |
| 51 | 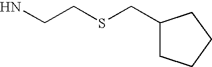 | |
| 52 | 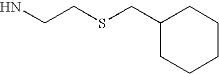 | |
| 53 | 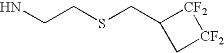 | |
| 54 | 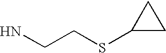 | |
| 55 | 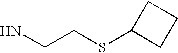 | |
| 56 | 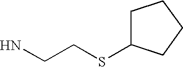 | 64 |
| 57 | 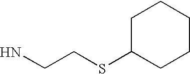 | |
| 58 | 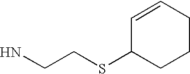 | |
| 59 | 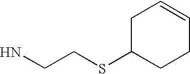 | |
| 60 | 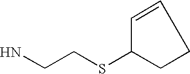 | |
| 61 | 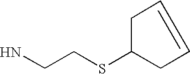 | |
| 62 | 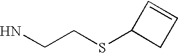 | |
| 63 | 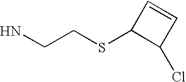 | |
| 64 | 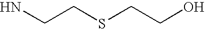 | oil, NMR (CDCl3): 3.79(q, 2H, CH₂OH); 2.35(tr, 1H, OH) |
| 65 | 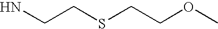 | |
| 66 | 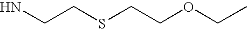 | oil, NMR (CDCl3): 3.48(q, 2H, CH₂CH₃); 1.13(tr, 3H, CH₃) |
| 67 | 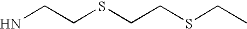 | |
| 68 | 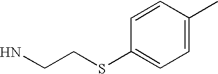 | 61 |
| 69 | 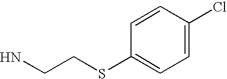 | |
| 70 | 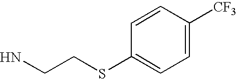 | 124-125 |
| 71 | 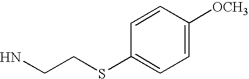 | 64-65 |
| 72 | 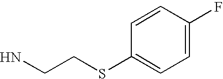 | |
| 73 | 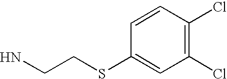 | |
| 74 | 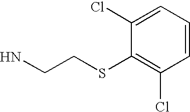 | |
| 75 | 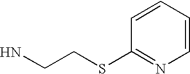 | |
| 76 | 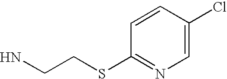 | |
| 77 | 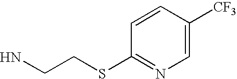 | 121-122 |
| 78 | 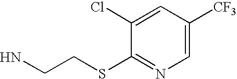 | |
| 79 | 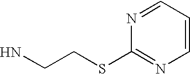 | 135 |
| 80 | 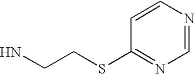 | |
| 81 | 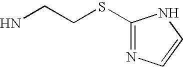 | |
| 82 | 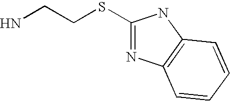 | |
| 83 | 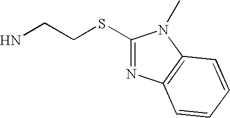 | |
| 84 | 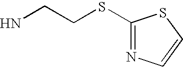 | |
| 85 | 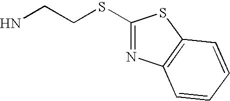 | |
| 86 | 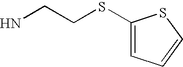 | |
| 87 | 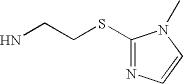 | |
| 88 | 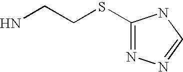 | |
| 89 | 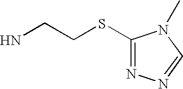 | |
| 90 | 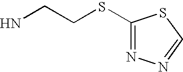 | |
| 91 | 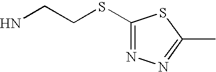 | |
| 92 | 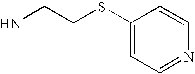 | resin, NMR (CDCl3): 3.71(q, 2H, CH₂NH); 3.28(tr, 2H, CH₂S) |
| 93 | 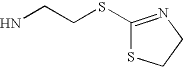 | |
| 94 | 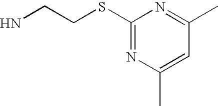 | 108-109 |
| 95 | 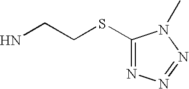 | 100-101 |
| 96 | 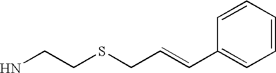 | |
| 97 | 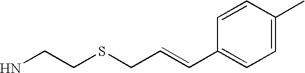 | |
| 98 | 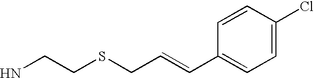 | |
| 99 | 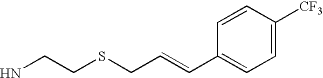 | |
| 100 | 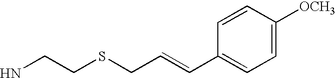 | |
| 101 | 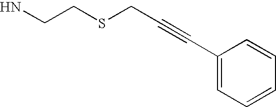 | |
| 102 | 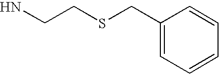 | 86-88 |
| 103 | 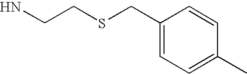 | |
| 104 | 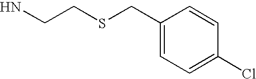 | |
| 105 | 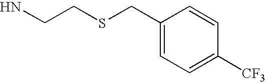 | |
| 106 | 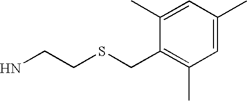 | |
| 107 | 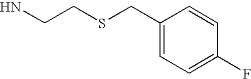 | |
| 108 | 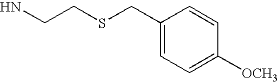 | |
| 109 | 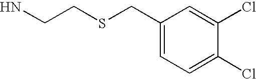 | |
| 110 | 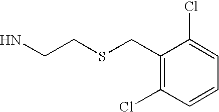 | |
| 111 | 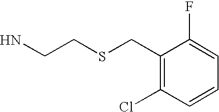 | 118-120 |
| 112 | 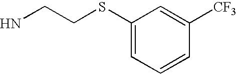 | |
| 113 | 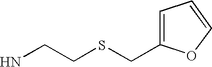 | 79-81 |
| 114 | 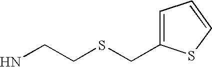 | 69-70 |
| 115 | 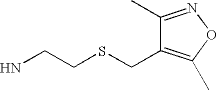 | |
| 116 | 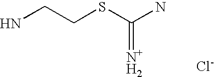 | 205 |
| 117 | 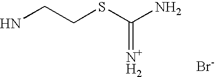 | |
| 118 | 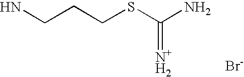 | |
| 119 | 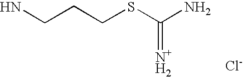 | |
| 120 | 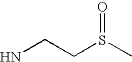 | 113-115 |
| 121 | 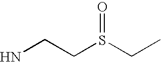 | 53-56 |
| 122 | 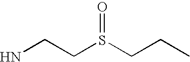 | |
| 123 | 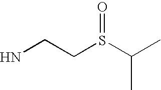 | |
| 124 | 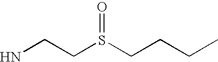 | |
| 125 | 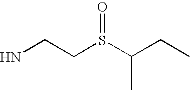 | |
| 126 | 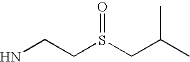 | |
| 127 | 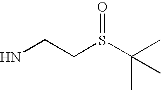 | |
| 128 | 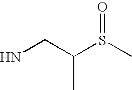 | |
| 129 | 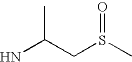 | |
| 130 | 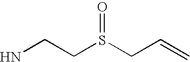 | |
| 131 | 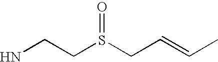 | |
| 132 | 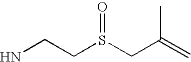 | 105-108 |
| 133 | 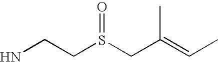 | |
| 134 | 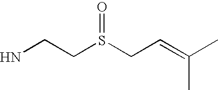 | 63-65 |
| 135 | 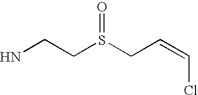 | |
| 136 | 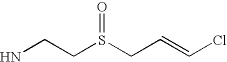 | |
| 137 | 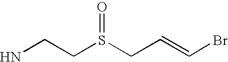 | oil, NMR (CDCl3): 6.69(d, 1H, CHBr); 6.32 (q, 1H, CH₂CH=) |
| 138 | 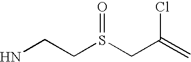 | |
| 139 | 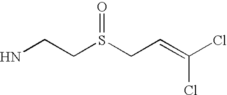 | |
| 140 | 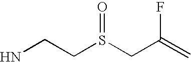 | |
| 141 | 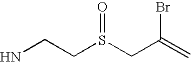 | |
| 142 | 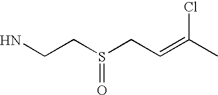 | 105-107 |
| 143 | 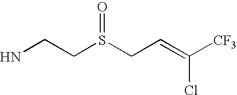 | |
| 145 | 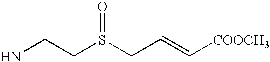 | |
| 146 | 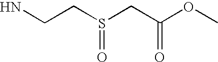 | |
| 147 | 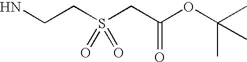 | |
| 148 | 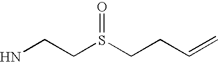 | |
| 149 | 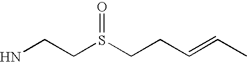 | |
| 150 | 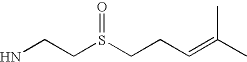 | |
| 151 | 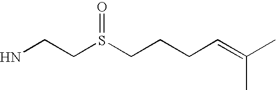 | |
| 152 | 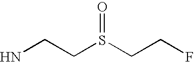 | |
| 153 | 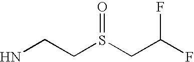 | |
| 154 | 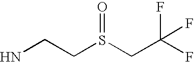 | |
| 156 | 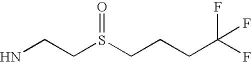 | |
| 157 | 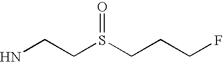 | |
| 158 | 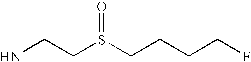 | |
| 159 | 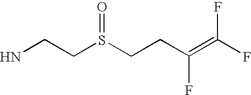 | |
| 160 | 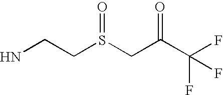 | |
| 161 | 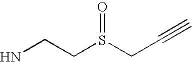 | |
| 162 | 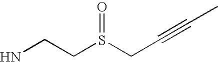 | |
| 163 | 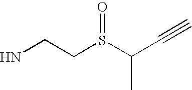 | |
| 164 | 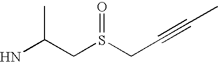 | |
| 165 | 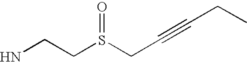 | |
| 166 | 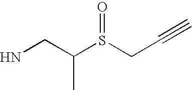 | |
| 167 | 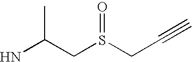 | |
| 168 | 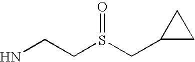 | |
| 169 | 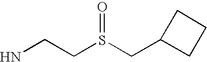 | 125-127 |
| 170 | 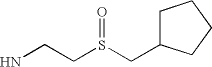 | |
| 171 | 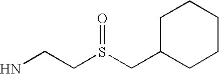 | |
| 172 | 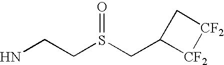 | |
| 173 | 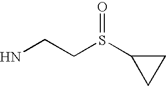 | |
| 174 | 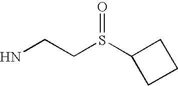 | |
| 175 | 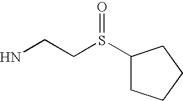 | |
| 176 | 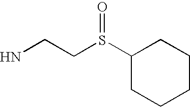 | |
| 177 | 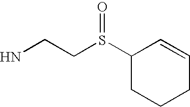 | |
| 178 | 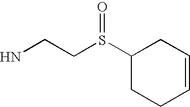 | |
| 179 | 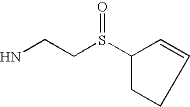 | |
| 180 | 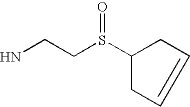 | |
| 181 | 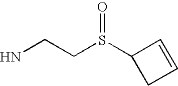 | |
| 182 | 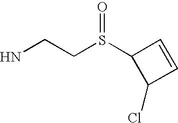 | |
| 183 | 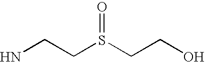 | |
| 184 | 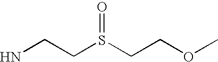 | |
| 185 | 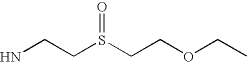 | |
| 186 | 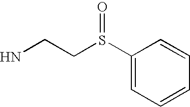 | |
| 187 | 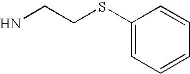 | |
| 188 | 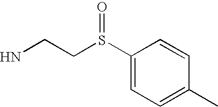 | |
| 189 | 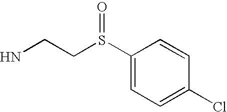 | |
| 190 | 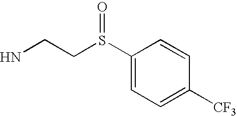 | 142 |
| 191 | 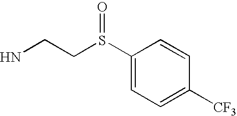 | 96 |
| 192 | 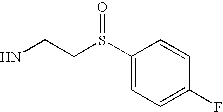 | |
| 193 | 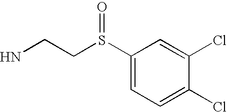 | |
| 194 | 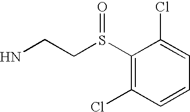 | |
| 195 | 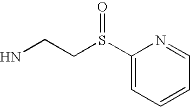 | |
| 196 | 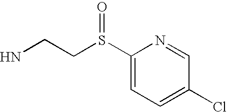 | |
| 197 | 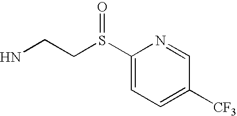 | |
| 198 | 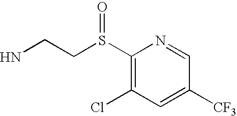 | |
| 199 | 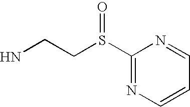 | |
| 200 | 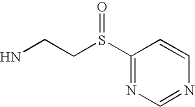 | |
| 201 | 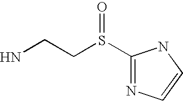 | |
| 202 | 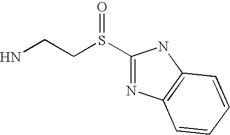 | |
| 203 | 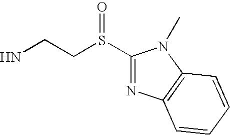 | |
| 204 | 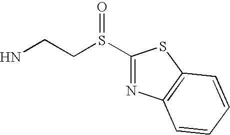 | |
| 205 | 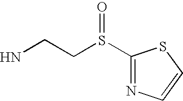 | |
| 206 | 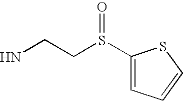 | |
| 207 | 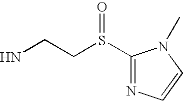 | |
| 208 | 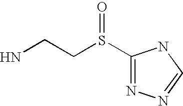 | |
| 209 | 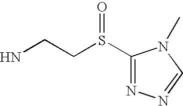 | |
| 210 | 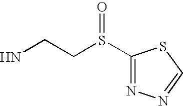 | |
| 211 | 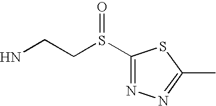 | |
| 212 | 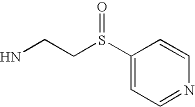 | |
| 213 | 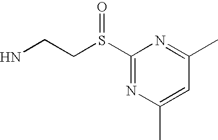 | oil, NMR (CDCl3): 3.57, 3.33(2m, 2H, CH₂SO); 2.54(s, 6H, 2CH₃) |
| 214 | 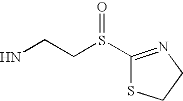 | |
| 215 | 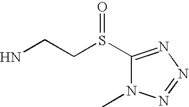 | oil, NMR 3H, OH₃); 3.90, 3.75(2m, 2H, CH₂SO) |
| 216 | 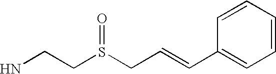 | |
| 217 | 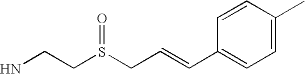 | |
| 218 | 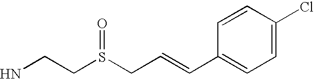 | |
| 219 | 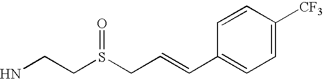 | |
| 220 | 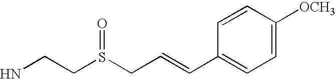 | |
| 221 | 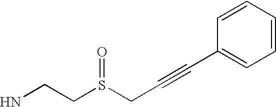 | |
| 222 | 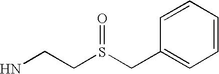 | 130-132 |
| 223 | 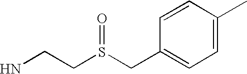 | |
| 224 | 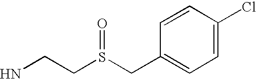 | |
| 225 | 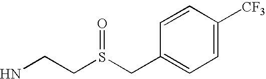 | |
| 226 | 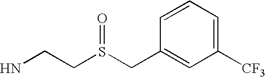 | |
| 227 | 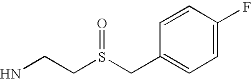 | |
| 228 | 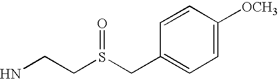 | |
| 229 | 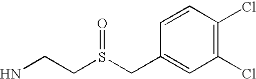 | |
| 230 | 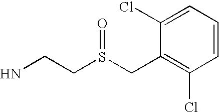 | |
| 231 | 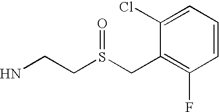 | 118-120 |
| 232 | 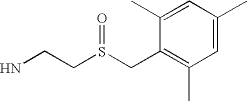 | |
| 233 | 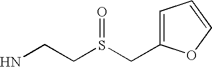 | 110-111 |
| 234 | 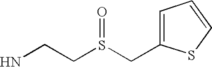 | 142 |
| 235 | 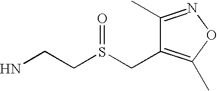 | |
| 236 | 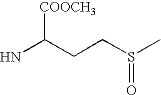 | |
| 237 | 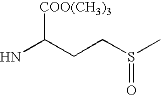 | |
| 238 | 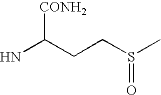 | |
| 239 | 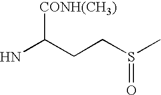 | |
| 240 | 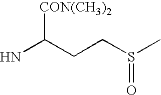 | |
| 241 | 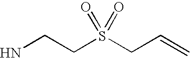 | |
| 242 | 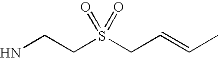 | 64-66 |
| 243 | 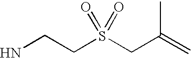 | 101-103 |
| 244 | 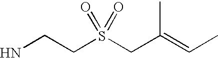 | 101 -103 |
| 245 | 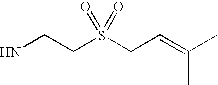 | 92-94 |
| 246 | 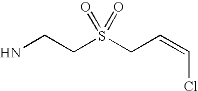 | 103-106 |

| Ex. No. | NR⁴R⁵ | m.p. [° C.] |
|---|---|---|
| 247 | 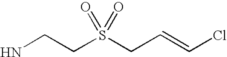 | |
| 248 | 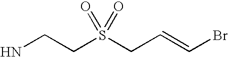 | 108-110 |
| 249 | 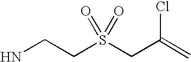 | 105-108 |
| 250 | 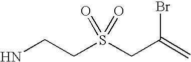 | 117-121 |
| 251 | 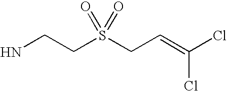 | 110-113 |
| 252 | 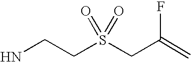 | |
| 253 | 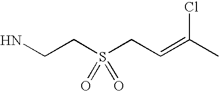 | 93-95 |
| 254 | 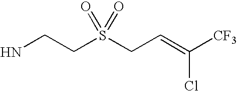 | |
| 255 | 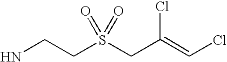 | |
| 256 | 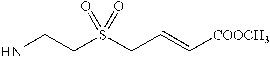 | |
| 257 | 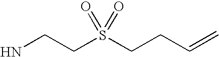 | |
| 258 | 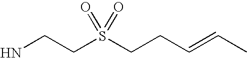 | |
| 259 | 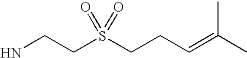 | 92-94 |
| 260 | 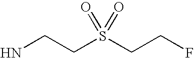 | |
| 261 | 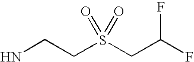 | |
| 262 | 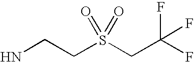 | 162 |
| 263 | 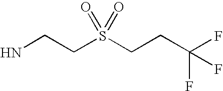 | |
| 264 | 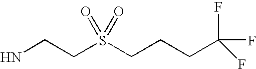 | |
| 265 | 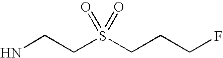 | |
| 266 | 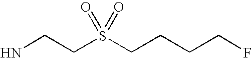 | |
| 267 | 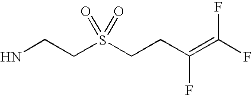 | |
| 268 | 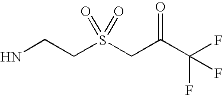 | |
| 269 | 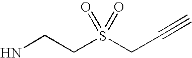 | |
| 270 | 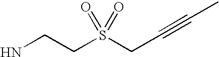 | |
| 271 | 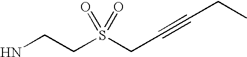 | |
| 272 | 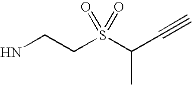 | |
| 273 | 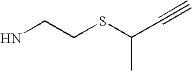 | |
| 274 | 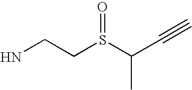 | |
| 275 | 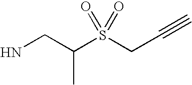 | |
| 276 | 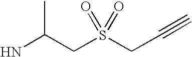 | |
| 277 | 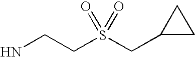 | |
| 278 | 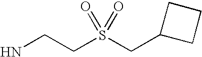 | 110-112 |
| 279 | 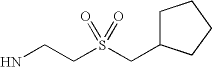 | |
| 280 | 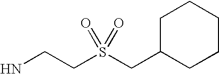 | |
| 281 | 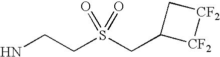 | |
| 282 | 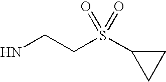 | |
| 283 | 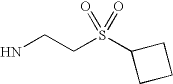 | |
| 284 | 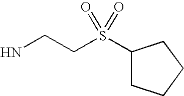 | 132 |
| 285 | 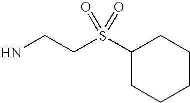 | |
| 286 | 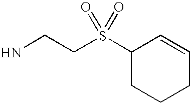 | |
| 287 | 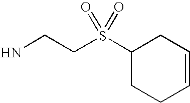 | |
| 288 | 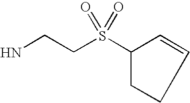 | |
| 289 | 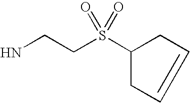 | |
| 290 | 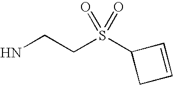 | |
| 291 | 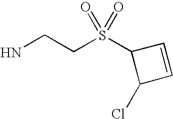 | |
| 292 | 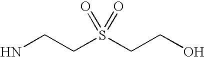 | |
| 293 | 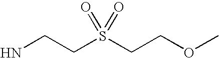 | |
| 294 | 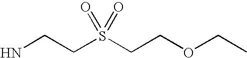 | |
| 295 | 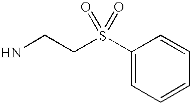 | 113-115 |
| 296 | 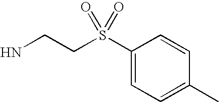 | 138 |
| 297 | 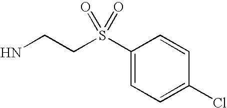 | |
| 298 | 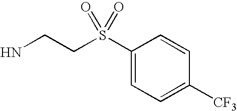 | 138 |
| 299 | 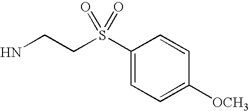 | 156 |
| 300 | 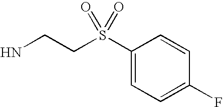 | |
| 301 | 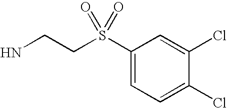 | |
| 302 | 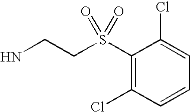 | |
| 303 | 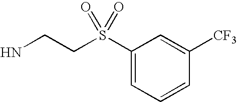 | |
| 304 | 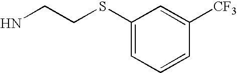 | |
| 305 | 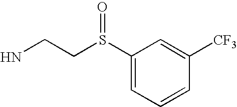 | |
| 306 | 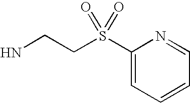 | |
| 307 | 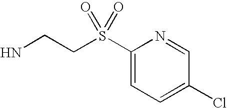 | |
| 308 | 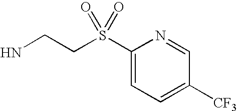 | 152-154 |
| 309 | 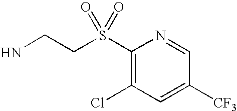 | |
| 310 | 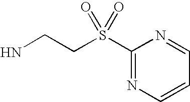 | 132 |
| 311 | 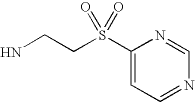 | |
| 312 | 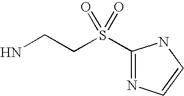 | |
| 313 | 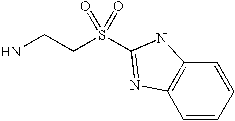 | |
| 314 | 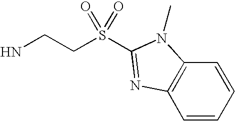 | |
| 315 | 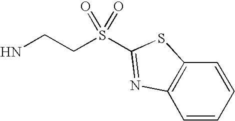 | |
| 316 | 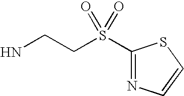 | |
| 317 | 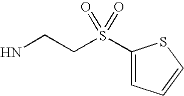 | |
| 318 | 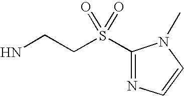 | |
| 319 | 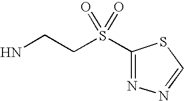 | |
| 320 | 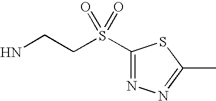 | |
| 321 | 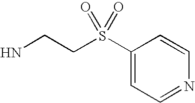 | 160 |
| 322 | 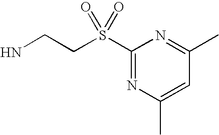 | 98 |
| 323 | 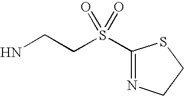 | |
| 324 | 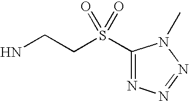 | 183-185 |
| 325 | 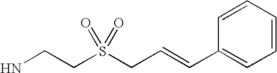 | |
| 326 | 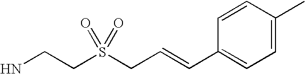 | |
| 327 | 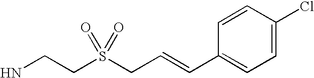 | |
| 328 | 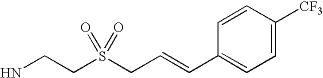 | |
| 329 | 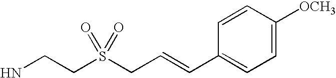 | |
| 330 | 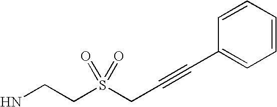 | |
| 331 | 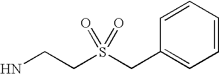 | 144-146 |
| 332 | 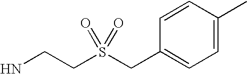 | |
| 333 | 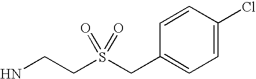 | |
| 334 | 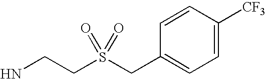 | |
| 335 | 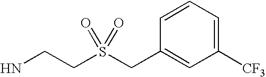 | |
| 336 | 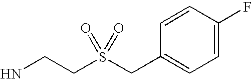 | |
| 337 | 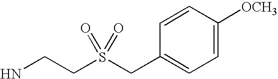 | |
| 338 | 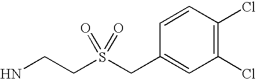 | |
| 339 | 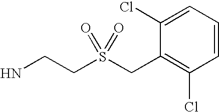 | |
| 340 | 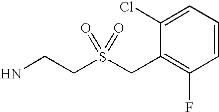 | 147-148 |
| 341 | 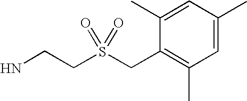 | |
| 342 | 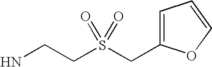 | 131-132 |
| 343 | 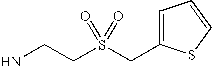 | 135-136 |
| 344 | 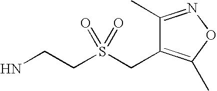 | |
| 345 | 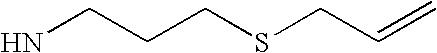 | oil, NMR(CDCl3): 5.78(m, 1H, CH₂CH═); 5.11(m, 2H, CH═CH₂); 3.13(d, 2H, SCH₂CH═) |
| 346 | 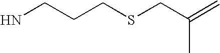 | |
| 347 | 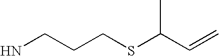 | |
| 348 | 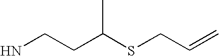 | |
| 349 | 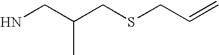 | |
| 350 | 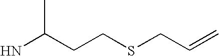 | |
| 351 | 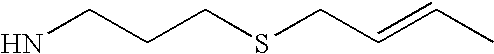 | |
| 352 | 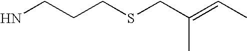 | |
| 353 | 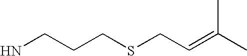 | |
| 354 | 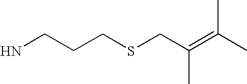 | |
| 355 | 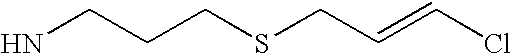 | |
| 356 | 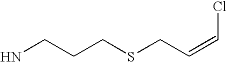 | |
| 357 | 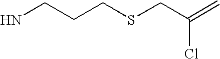 | |
| 358 | 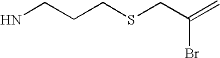 | |
| 359 | 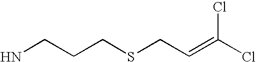 | |
| 360 | 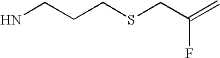 | |
| 361 | 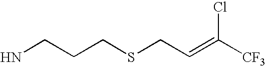 | |
| 362 | 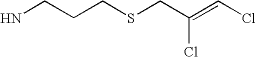 | |
| 363 | 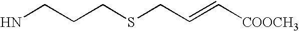 | |
| 364 | 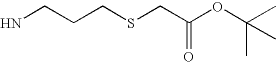 | |
| 365 | 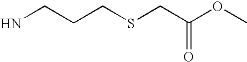 | |
| 365a | 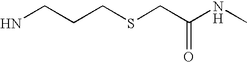 | 123-124 |
| 366 | 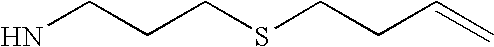 | |
| 367 | 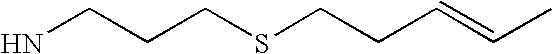 | |
| 368 | 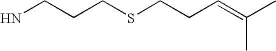 | |
| 369 | 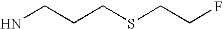 | |
| 370 | 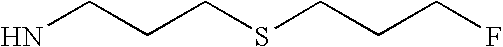 | |
| 371 | 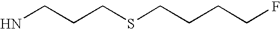 | |
| 372 | 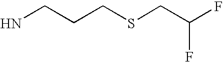 | |
| 373 | 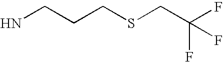 | 83-84 |
| 374 | 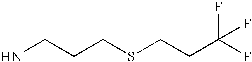 | |
| 375 | 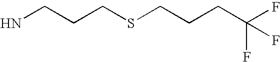 | |
| 376 | 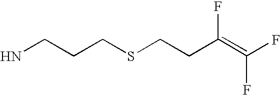 | |
| 377 | 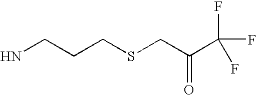 | |
| 378 | 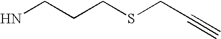 | |
| 379 | 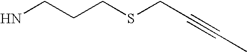 | |
| 380 | 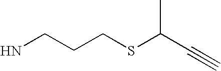 | |
| 381 | 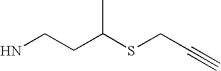 | |
| 382 | 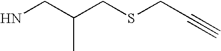 | |
| 383 | 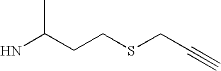 | |
| 384 | 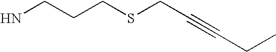 | |
| 385 | 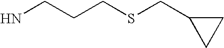 | |
| 386 | 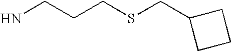 | |
| 387 | 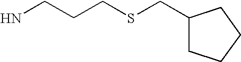 | |
| 388 | 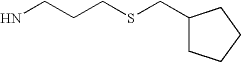 | |
| 389 | 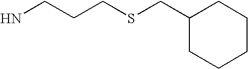 | |
| 390 | 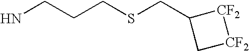 | |
| 391 | 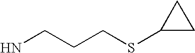 | |
| 392 | 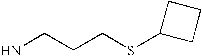 | |
| 393 | 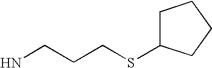 | 71-73 |
| 394 | 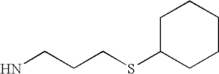 | |
| 395 | 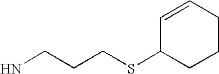 | |
| 396 | 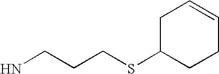 | |
| 397 | 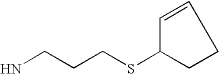 | |
| 398 | 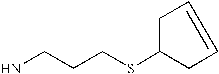 | |
| 399 | 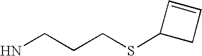 | |
| 400 | 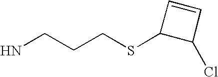 | |
| 401 | 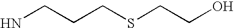 | |
| 402 | 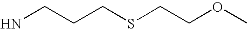 | |
| 403 | 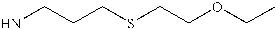 | |
| 404 | 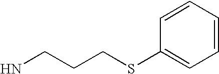 | 90 |
| 404 | 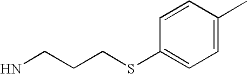 | 97 |
| 406 | 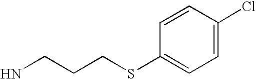 | |
| 407 | 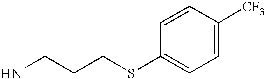 | 144-145 |
| 408 | 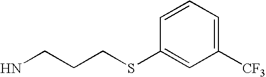 | |
| 409 | 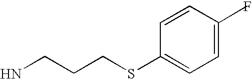 | 112 |
| 409a | 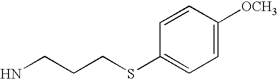 | 97-98 |
| 410 | 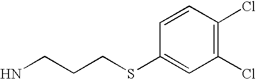 | |
| 411 | 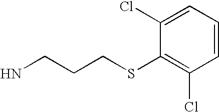 | |
| 412 | 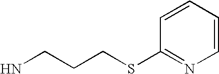 | |
| 413 | 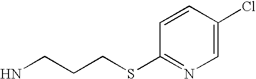 | |
| 414 | 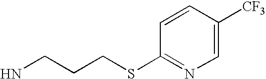 | 110 |
| 415 | 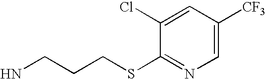 | |
| 416 | 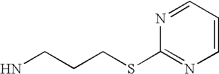 | 119 |
| 417 | 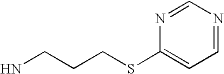 | |
| 418 | 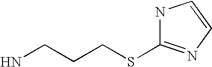 | |
| 419 | 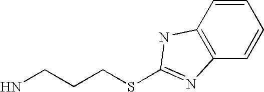 | |
| 420 | 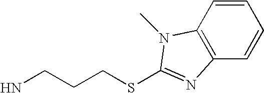 | oil, NMR(CDCl3): 3.65(s, 1H, CH₃); 3.53(tr, 2H, CH₂S) |
| 421 | 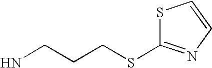 | oil, NMR(CDCl3): 7.35, 7.18(2d, 2H, thiazole-H), 3.53(tr, 2H, CH₂S) |
| 422 | 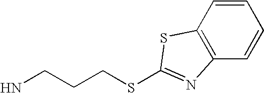 | 143 |
| 423 | 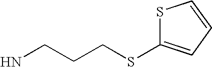 | 54 |
| 424 | 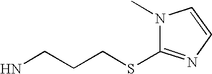 | oil, NMR(CDCl3): 3.55(tr, 2H, CH₂S); 3.23(tr, 2H, CH₂S) |
| 425 | 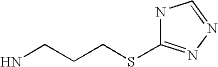 | |
| 426 | 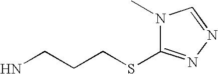 | oil, NMR(CDCl3): 3.57(tr, 2H, CH₂S); 3.35(tr, 2H, CH₂S) |
| 427 | 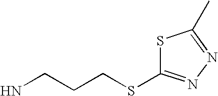 | oil, NMR(CDCl3): 3.40(tr, 2H, CH₂S) 2.67(s, 1H, CH₃); |
| 428 | 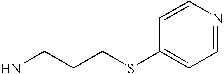 | 94 |
| 429 | 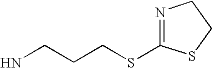 | oil, NMR(CDCl3): 3.34(tr, 2H, CH₂S); 3.22(tr, 2H, CH₂S) |
| 430 | 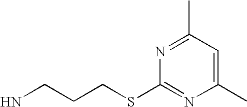 | 110-111 |
| 431 | 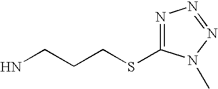 | oil, NMR(CDCl3): 3.92(s, 1H, CH₃); 3.43(tr, 2H, CH₂S) |
| 432 | 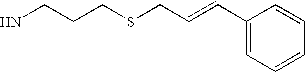 | |
| 433 | 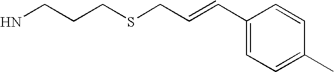 | |
| 434 | 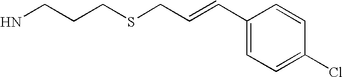 | |
| 435 | 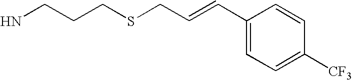 | |
| 436 | 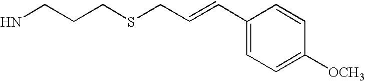 | |
| 437 | 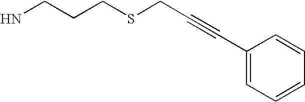 | |
| 438 | 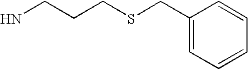 | 88 |
| 439 | 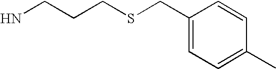 | |
| 440 | 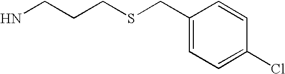 | |
| 441 | 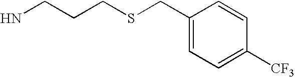 | |
| 442 | 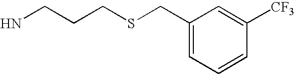 | |
| 443 | 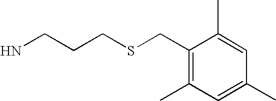 | |
| 444 | 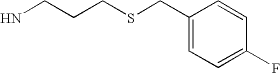 | |
| 445 | 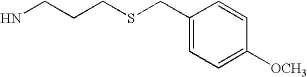 | |
| 446 | 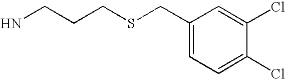 | |
| 447 | 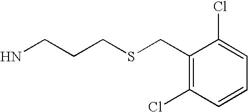 | |
| 448 | 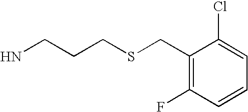 | |
| 449 | 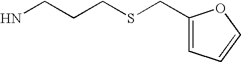 | 74 |
| 450 | 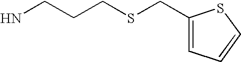 | 88 |
| 451 | 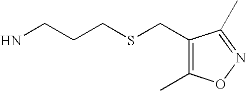 | |
| 452 | 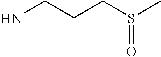 | oil, NMR(CDCl₃): 2.82(m, 2H, CH₂SO); 2.58(s, 3H, CH₃) |
| 453 | 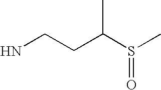 | |
| 454 | 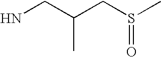 | |
| 455 | 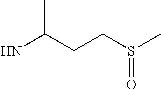 | |
| 456 | 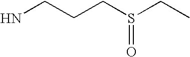 | 79-81 |
| 457 | 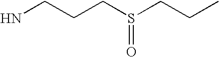 | |
| 458 | 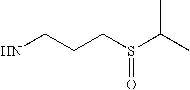 | 81 |
| 459 | 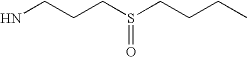 | |
| 460 | 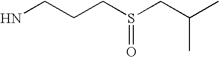 | |
| 461 | 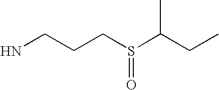 | |
| 462 | 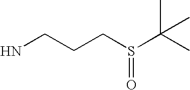 | 120-121 |
| 463 | 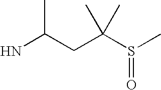 | oil, NMR(CDCl₃): 4.38(m, 1H, methine-H); 2.04(S, 3H, SCH₃); 1.37(s, 6H, C(CH₃)₂ |
| 464 | 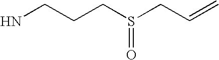 | 118 |
| 465 | 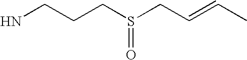 | |
| 466 | 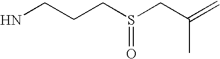 | 105-108 |
| 467 | 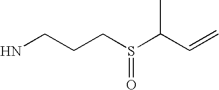 | |
| 468 | 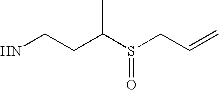 | |
| 469 | 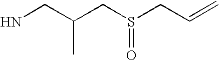 | |
| 470 | 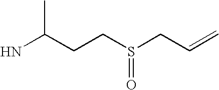 | |
| 471 | 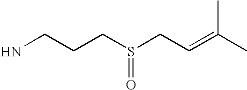 | |
| 472 | 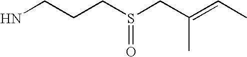 | |
| 473 | 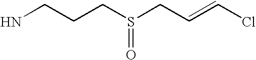 | |
| 474 | 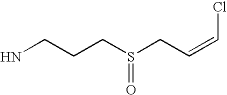 | |
| 475 | 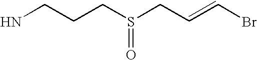 | |
| 476 | 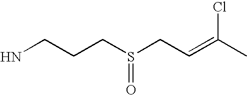 | |
| 477 | 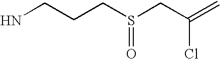 | |
| 478 | 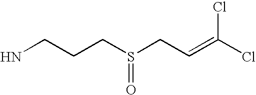 | |
| 479 | 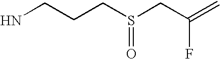 | |
| 480 | 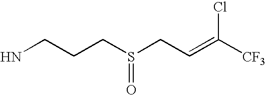 | |
| 481 | 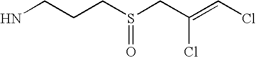 | |
| 482 | 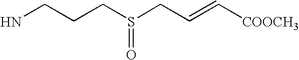 | |
| 483 | 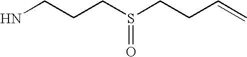 | |
| 484 | 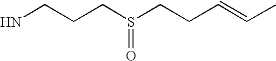 | |
| 485 | 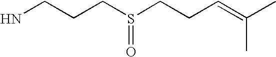 | |
| 486 | 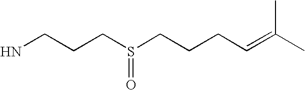 | |
| 487 | 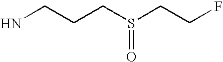 | |
| 488 | 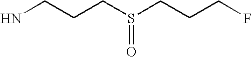 | |
| 489 | 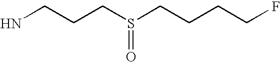 | |

| Ex. No. | NR⁴R⁵ | m.p.[° C.] |
|---|---|---|
| 490 | 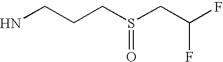 | |
| 491 | 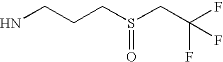 | 140 |
| 492 | 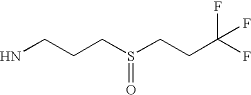 | |
| 493 | 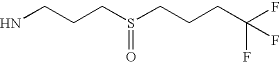 | |
| 494 | 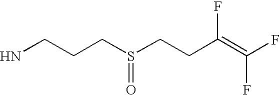 | |
| 495 | 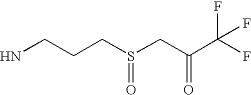 | |
| 496 | 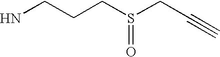 | |
| 497 | 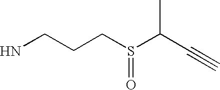 | |
| 498 | 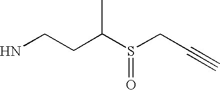 | |
| 499 | 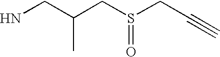 | |
| 500 | 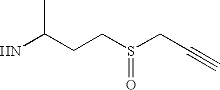 | |
| 501 | 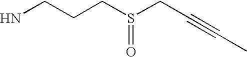 | |
| 502 | 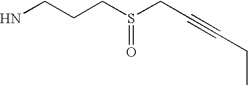 | |
| 503 | 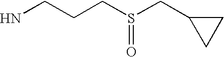 | |
| 504 | 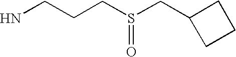 | 125-127 |
| 505 | 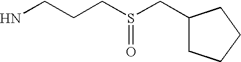 | |
| 506 | 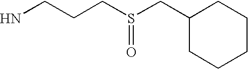 | |
| 507 | 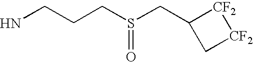 | |
| 508 | 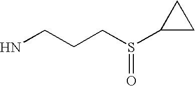 | |
| 509 | 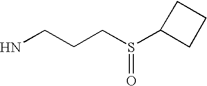 | |
| 510 | 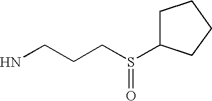 | oil, NMR(CDCl₃): 3.0(m, 1H, methine-H); 3.76, 3.69(2m, 2H, CH₂SO) |
| 511 | 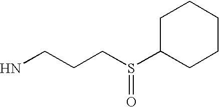 | |
| 512 | 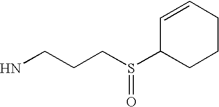 | |
| 513 | 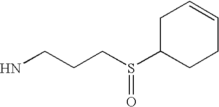 | |
| 514 | 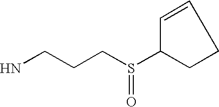 | |
| 515 | 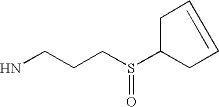 | |
| 516 | 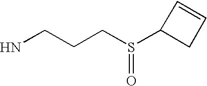 | |
| 517 | 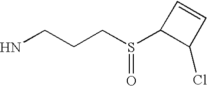 | |
| 518 | 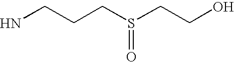 | |
| 519 | 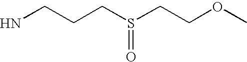 | |
| 520 | 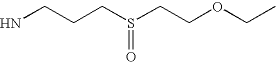 | |
| 521 | 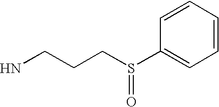 | 78 |
| 522 | 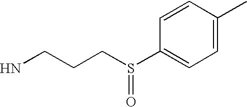 | 78 |
| 523 | 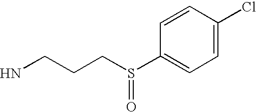 | |
| 524 | 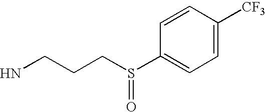 | |
| 525 | 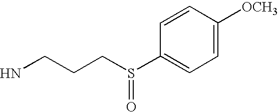 | oil, NMR(CDCl₃): 4.85(s, 3H, OCH₃); 3.58(q, 2H, CH₂NH); 2.89(m, 2H, CH₂SO) |
| 526 | 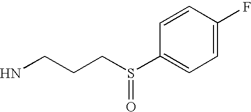 | 98 |
| 527 | 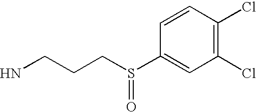 | |
| 528 | 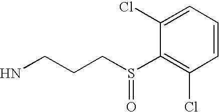 | |
| 529 | 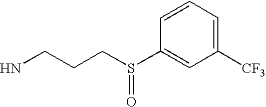 | |
| 530 | 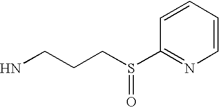 | |
| 531 | 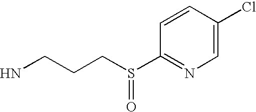 | |
| 532 | 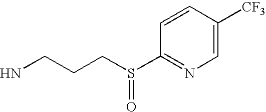 | |
| 533 | 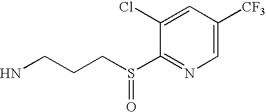 | |
| 534 | 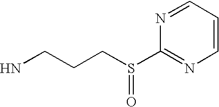 | oil, NMR(CDCl₃): 8.88(d, 2H, pyrimidine-H); 7.42(tr, 1H, pyrimidine-H); 3.29(m, 2H, CH₂SO) |
| 535 | 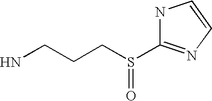 | |
| 536 | 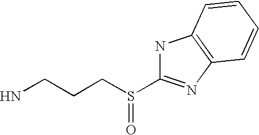 | |
| 537 | 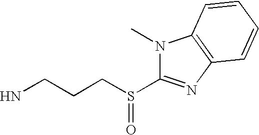 | 123 |
| 538 | 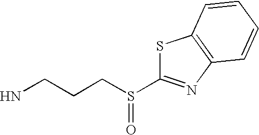 | |
| 539 | 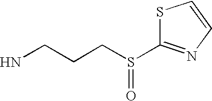 | |
| 540 | 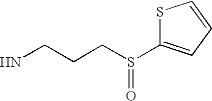 | |
| 541 | 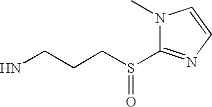 | oil, NMR(CDCl₃): 7.01(d, 2H, imidazole-H); 3.91(s, 3H, CH₃); 3.51(m, 2H, CH₂SO) |
| 542 | 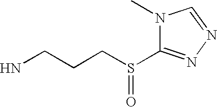 | |
| 543 | 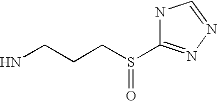 | |
| 544 | 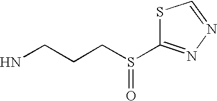 | |
| 545 | 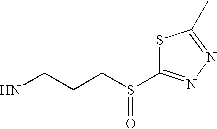 | 94 |
| 546 | 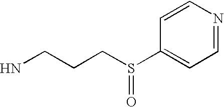 | oil, NMR(CDCl₃): 8.76, 7.48(2d, 4H, pyridine-H); 3.18, 2.88(2m, 2H, CH₂SO) |
| 547 | 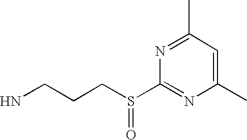 | oil, NMR(CDCl₃): 7.10(s, 1H, pyrimidine-H); 3.23(m, 2H, CH₂SO); 2.57(s, 6H, 2 CH₃) |
| 548 | 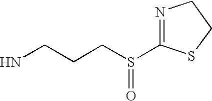 | |
| 549 | 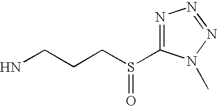 | |
| 550 | 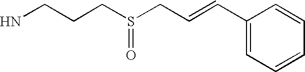 | |
| 551 | 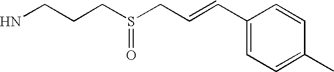 | |
| 552 | 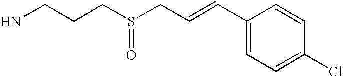 | |
| 553 | 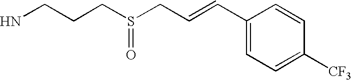 | |
| 554 | 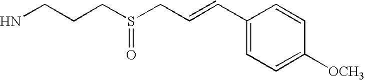 | |
| 555 | 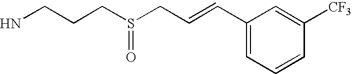 | |
| 556 | 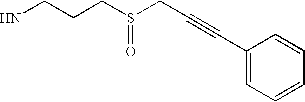 | |
| 557 | 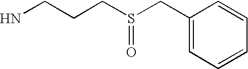 | 136 |
| 558 | 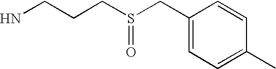 | |
| 559 | 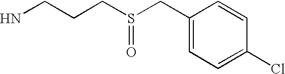 | |
| 560 | 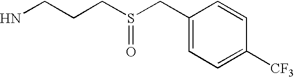 | |
| 561 | 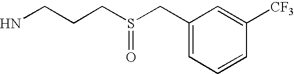 | |
| 562 | 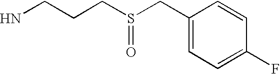 | |
| 563 | 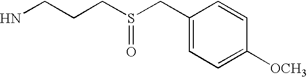 | |
| 564 | 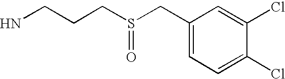 | |
| 565 | 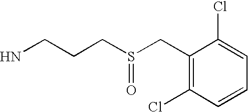 | |
| 566 | 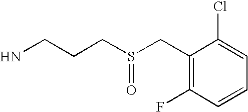 | |
| 567 | 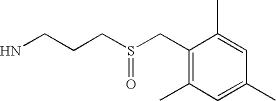 | |
| 568 | 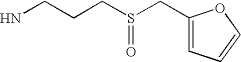 | 105-106 |
| 569 | 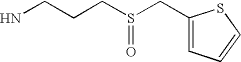 | 88-89 |
| 570 | 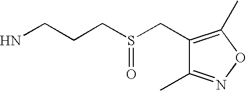 | |
| 571 | 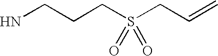 | 125-126 |
| 572 | 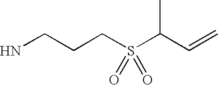 | |
| 573 | 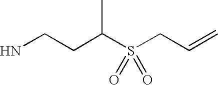 | |
| 574 | 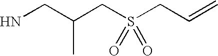 | |
| 575 | 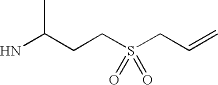 | |
| 576 | 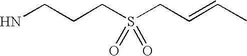 | |
| 577 | 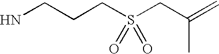 | 101-103 |
| 578 | 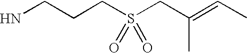 | |
| 579 | 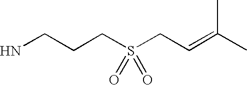 | |
| 580 | 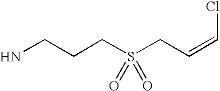 | |
| 581 | 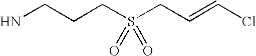 | |
| 582 | 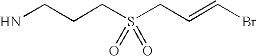 | |
| 583 | 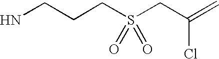 | |
| 584 | 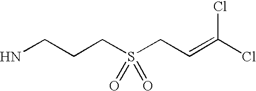 | 110-113 |
| 585 | 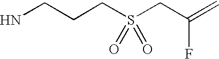 | |
| 586 | 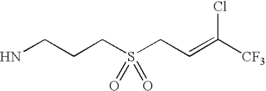 | |
| 587 | 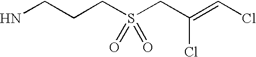 | |
| 588 | 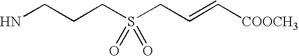 | |
| 589 | 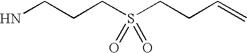 | |
| 590 | 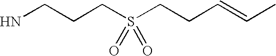 | |
| 591 | 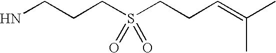 | |
| 592 | 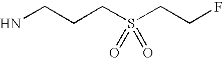 | |
| 593 | 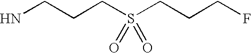 | |
| 594 | 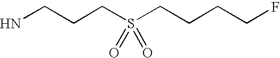 | |
| 595 | 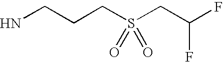 | |
| 596 | 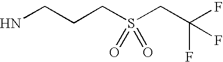 | 168 |
| 597 | 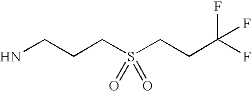 | |
| 598 | 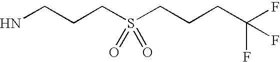 | |
| 599 | 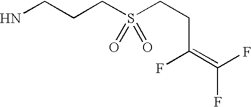 | |
| 600 | 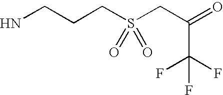 | |
| 601 | 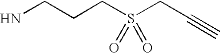 | |
| 602 | 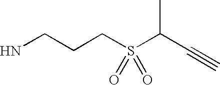 | |
| 603 | 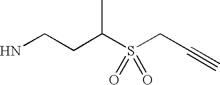 | |
| 604 | 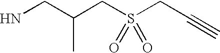 | |
| 605 | 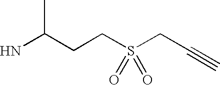 | |
| 606 | 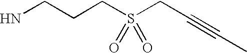 | |
| 607 | 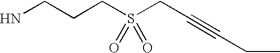 | |
| 608 | 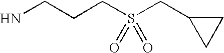 | |
| 609 | 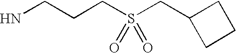 | 110-112 |
| 610 | 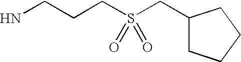 | |
| 611 | 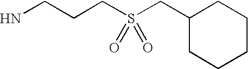 | |
| 612 | 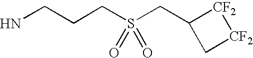 | |
| 613 | 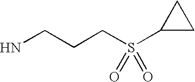 | |
| 614 | 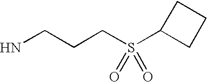 | |
| 615 | 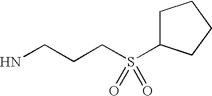 | 104 |
| 616 | 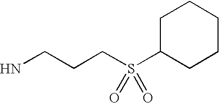 | |
| 617 | 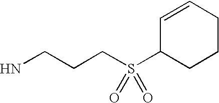 | |
| 618 | 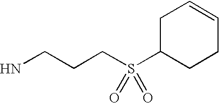 | |
| 619 | 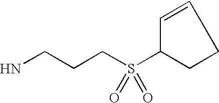 | |
| 620 | 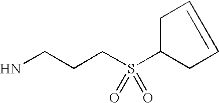 | |
| 621 | 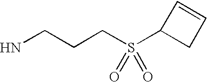 | |
| 622 | 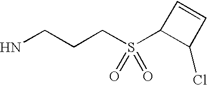 | |
| 623 | 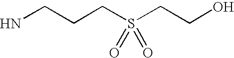 | |
| 624 | 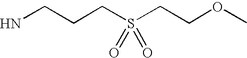 | |
| 625 | 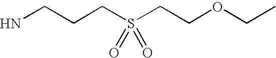 | |
| 626 | 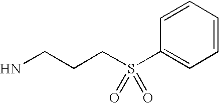 | 88 |
| 627 | 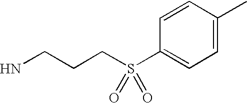 | |
| 628 | 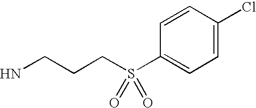 | |
| 629 | 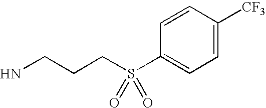 | 153 |
| 630 | 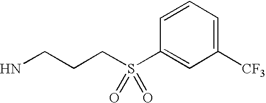 | |
| 631 | 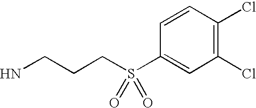 | |
| 632 | 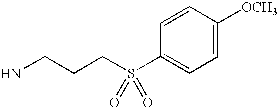 | oil, NMR(CDCl₃): 3.89(s, 3H, OCH₃); 3.18(tr, 2H, CH₂SO₂) |
| 633 | 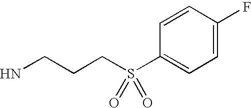 | 128 |
| 634 | 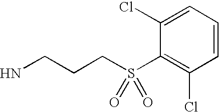 | |
| 635 | 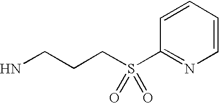 | |
| 636 | 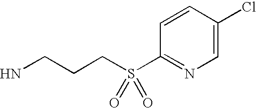 | |
| 637 | 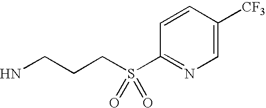 | 128 |
| 638 | 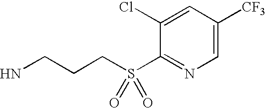 | |
| 639 | 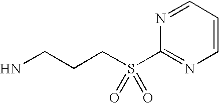 | oil, NMR(CDCl₃): 8,95(d, 2H, pyrimidine-H); 7.60(tr, 1H, pyrimidine-H); 3.65(m, 4H, CH₂NH, CH₂SO₂) |
| 640 | 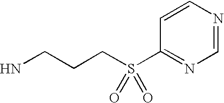 | |
| 641 | 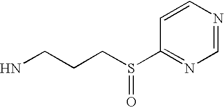 | |
| 642 | 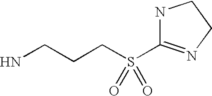 | |
| 643 | 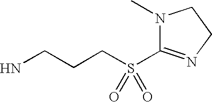 | |
| 644 | 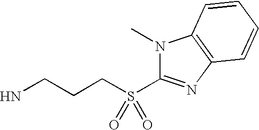 | 72 |
| 645 | 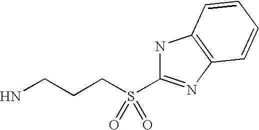 | |
| 646 | 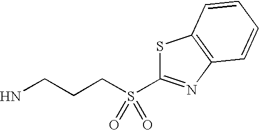 | 131-133 |
| 647 | 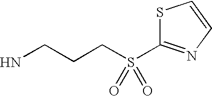 | |
| 648 | 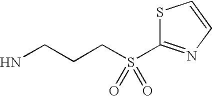 | |
| 649 | 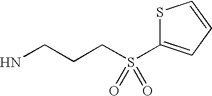 | 128 |
| 650 | 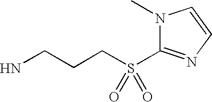 | |
| 651 | 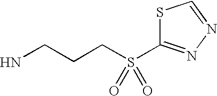 | |
| 652 | 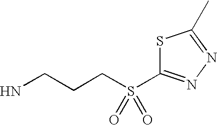 | 143-144 |
| 653 | 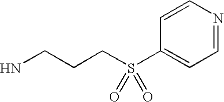 | 139 |
| 654 | 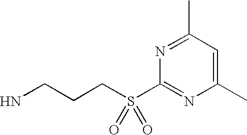 | oil, NMR(CDCl₃): 7.23(s,1H, pyrimidine-H); 3.62(tr, 2H, CH₂SO₂); 2.60 (s, 6H; 2CH₃) |
| 655 | 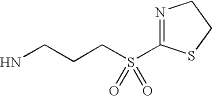 | |
| 656 | 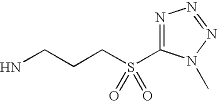 | |
| 657 | 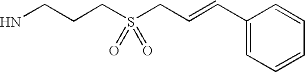 | |
| 658 | 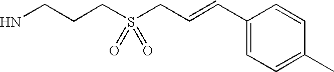 | |
| 659 | 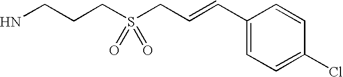 | |
| 660 | 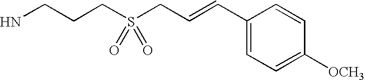 | |
| 661 | 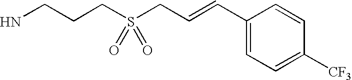 | |
| 662 | 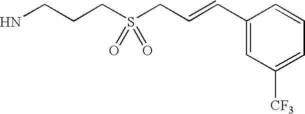 | |
| 663 | 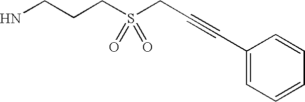 | |
| 634 | 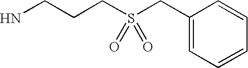 | 153 |
| 665 | 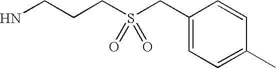 | |
| 666 | 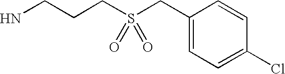 | |
| 667 | 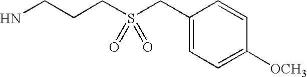 | |
| 668 | 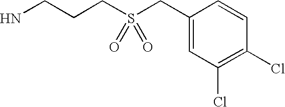 | |
| 669 | 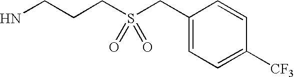 | |
| 670 | 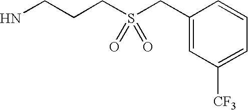 | |
| 671 | 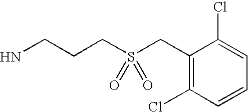 | |
| 672 | 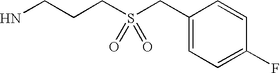 | |
| 673 | 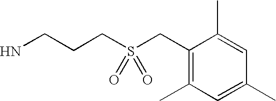 | |
| 674 | 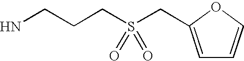 | 132 |
| 675 | 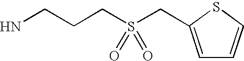 | 108-109 |
| 676 | 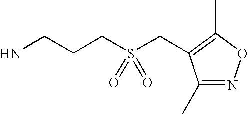 | |
| 677 | 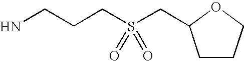 | |
| 678 | 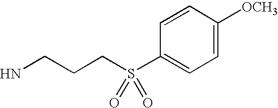 | |
| 679 | 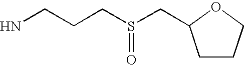 | |
| 680 | 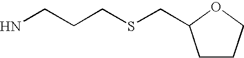 | |
| 681 | 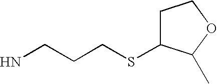 | 63 |
| 682 | 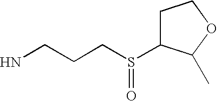 | 113-117 |
| 683 | 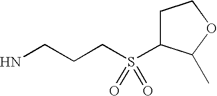 | 105-107 |
| 684 | 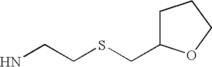 | |
| 685 | 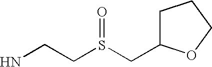 | |
| 686 | 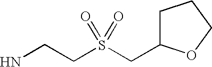 | |
| 687 | 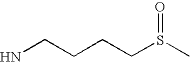 | 70-72 |
| 688 | 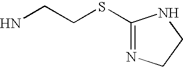 | |
| 689 | 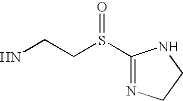 | |
| 690 | 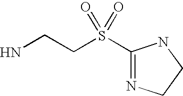 | |
| 691 | 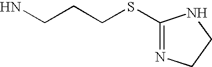 | |
| 692 | 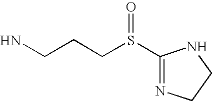 | oil, NMR(CDCl₃): 4.53, 4.30(2m, 2H, CH₂N); 3.25 (m, 2H, CH₂SO) |
| 693 | 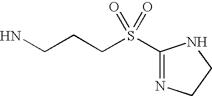 | |
| 694 | 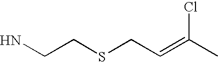 | oil, NMR(CDCl₃): 5.56(tr, 1H, olef. H); 2.31(d, CH₂CH); 2.17 (s, 3H, CH₃) |
| 695 | 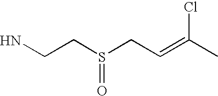 | 105-107 |
| 696 | 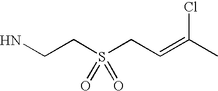 | 93-95 |
| 697 | 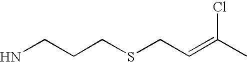 | |
| 698 | 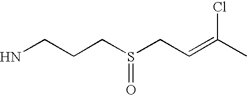 | |
| 699 | 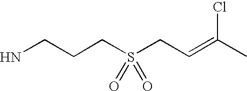 | |
| 700 | 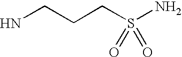 | |
| 701 | 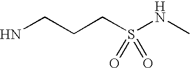 | |

| Ex. No. | NR⁴R⁵ | m.p.[° C.] |
|---|---|---|
| 702 | 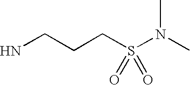 | |
| 703 | 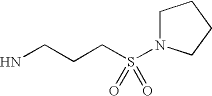 | 128 |
| 704 | 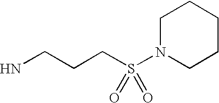 | 130 |
| 705 | 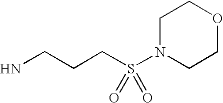 | 160 |
| 706 | 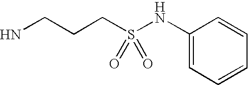 | |
| 707 | 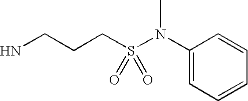 | |
| 708 | 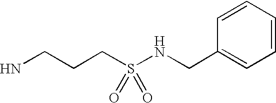 | |
| 709 | 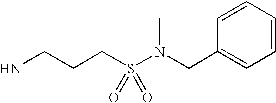 | |
| 710 | 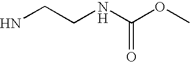 | 125-127 |
| 711 | 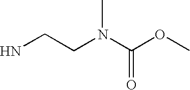 | |
| 712 | 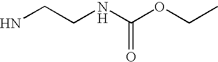 | 141 |
| 713 | 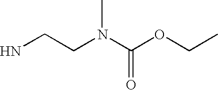 | |
| 714 | 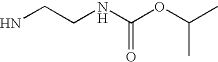 | |
| 715 | 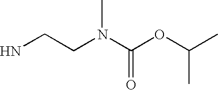 | |
| 716 | 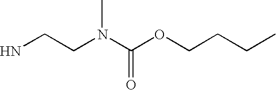 | |
| 717 | 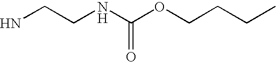 | |
| 718 | 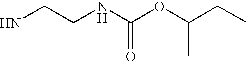 | |
| 719 | 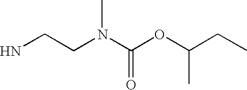 | |
| 720 | 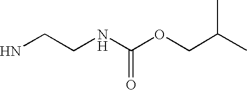 | |
| 721 | 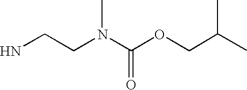 | |
| 722 | 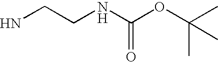 | 114-115 |
| 723 | 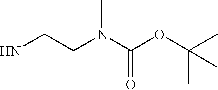 | oil, NMR(CDCl₃): 3.58, 3.39(2q, 4H, 2 CH₂NH); 1.40(s, 9H, 3 CH₃) |
| 724 | 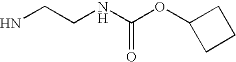 | |
| 725 | 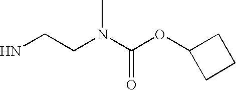 | |
| 726 | 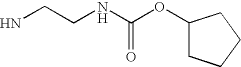 | |
| 727 | 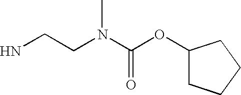 | |
| 728 | 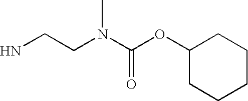 | |
| 729 | 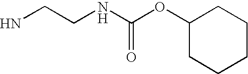 | |
| 730 | 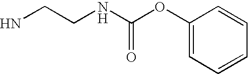 | |
| 731 | 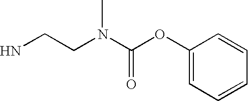 | |
| 732 | 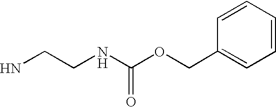 | |
| 733 | 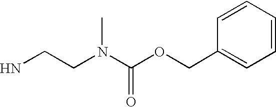 | |
| 734 | 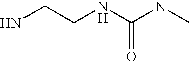 | |
| 735 | 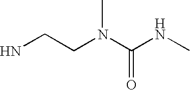 | |
| 736 | 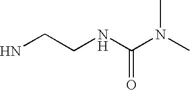 | |
| 737 | 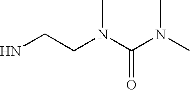 | |
| 738 | 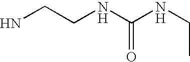 | 170-172 |
| 739 | 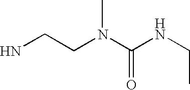 | |
| 740 | 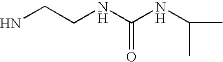 | 186-187 |
| 741 | 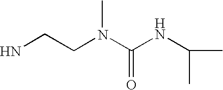 | |
| 742 | 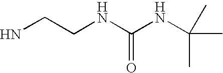 | |
| 743 | 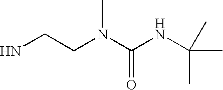 | |
| 744 | 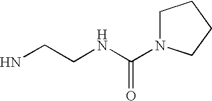 | 237-239 |
| 745 | 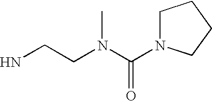 | |
| 746 | 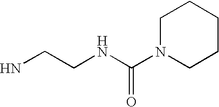 | |
| 747 | 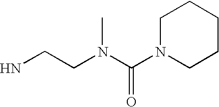 | |
| 748 | 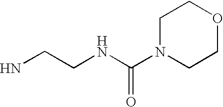 | |
| 749 | 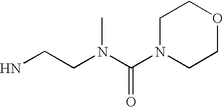 | |
| 750 | 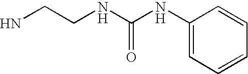 | |
| 751 | 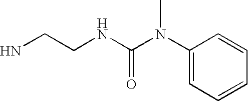 | |
| 752 | 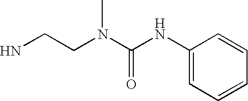 | |
| 753 | 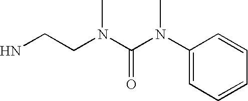 | |
| 754 | 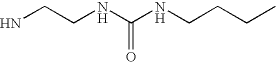 | oil, NMR(CDCl₃): 3.54, 3.43, 3.10 (3 q, 9H, 3 CH₂NH); 0.90(tr, 3H, CH₃) |
| 755 | 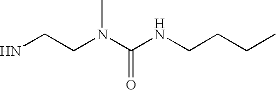 | |
| 756 | 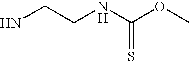 | |
| 757 | 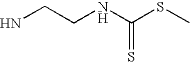 | 156 |
| 758 | 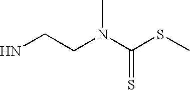 | |
| 759 | 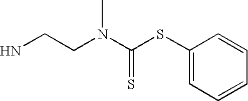 | |
| 760 | 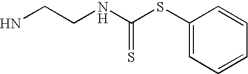 | |
| 761 | 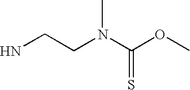 | |
| 762 | 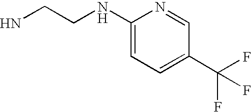 | |
| 763 | 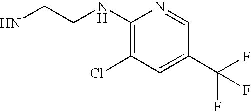 | |
| 764 | 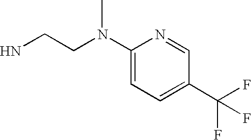 | |
| 765 | 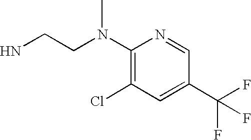 | |
| 766 | 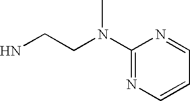 | |
| 767 | 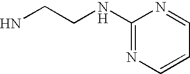 | |
| 768 | 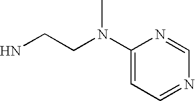 | |
| 769 | 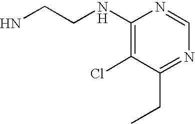 | |
| 770 | 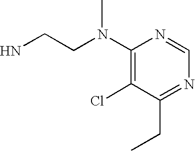 | |
| 771 | 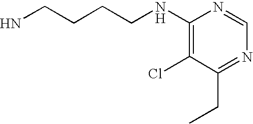 | oil, NMR(CDCl₃): 8.82(d, 1H pyridine-H); 8.80 (s, 1H, pyridine- H); 8.22(s, 1H, pyrimidine-H); 7.58(d, 1H, pyridine-H) |
| 772 | 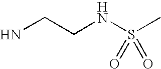 | |
| 773 | 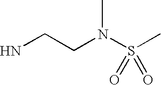 | |
| 774 | 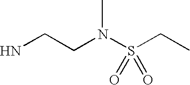 | |
| 775 | 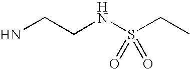 | |
| 776 | 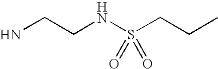 | |
| 777 | 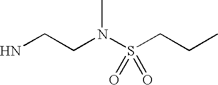 | |
| 778 | 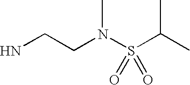 | |
| 779 | 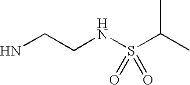 | |
| 780 | 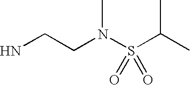 | |
| 781 | 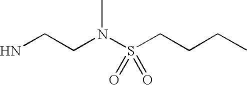 | |
| 782 | 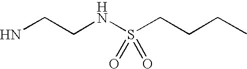 | |
| 783 | 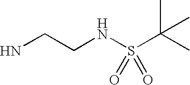 | |
| 784 | 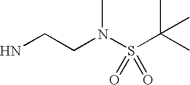 | |
| 785 | 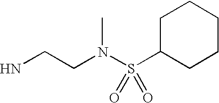 | |
| 786 | 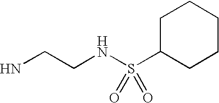 | |
| 787 | 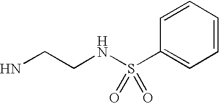 | |
| 788 | 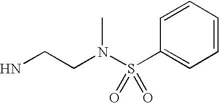 | |
| 789 | 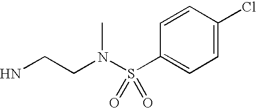 | |
| 790 | 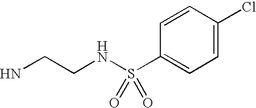 | |
| 791 | 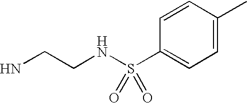 | |
| 792 | 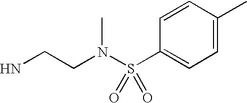 | |
| 793 | 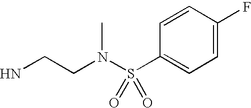 | |
| 794 | 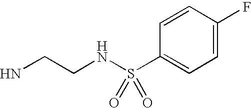 | |
| 795 | 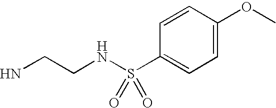 | |
| 796 | 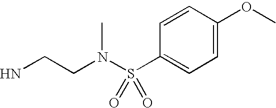 | |
| 797 | 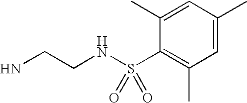 | |
| 798 | 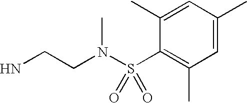 | |
| 799 | 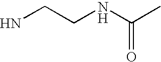 | 160 |
| 800 | 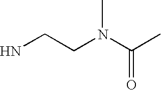 | |
| 801 | 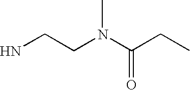 | |
| 802 | 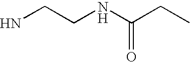 | |
| 803 | 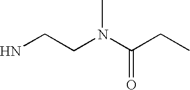 | |
| 804 | 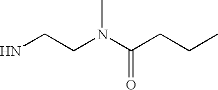 | |
| 805 | 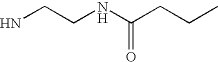 | |
| 806 | 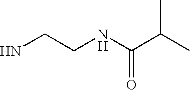 | |
| 807 | 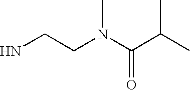 | |
| 808 | 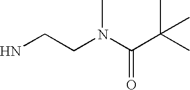 | |
| 809 | 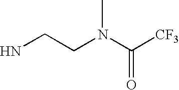 | |
| 810 | 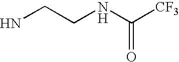 | |
| 811 | 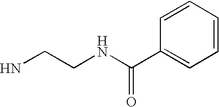 | |
| 812 | 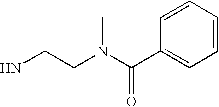 | |
| 813 | 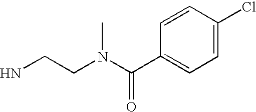 | |
| 814 | 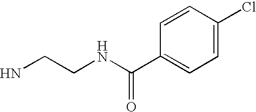 | |
| 815 | 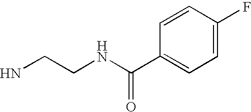 | |
| 816 | 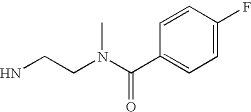 | |
| 817 | 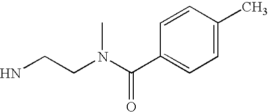 | |
| 818 | 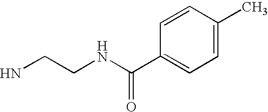 | |
| 819 | 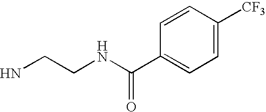 | |
| 820 | 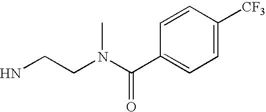 | |
| 821 | 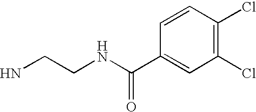 | |
| 822 | 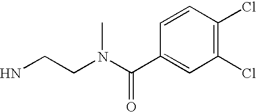 | |
| 823 | 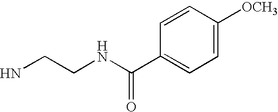 | |
| 824 | 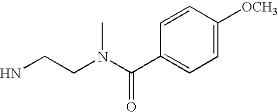 | |
| 825 | 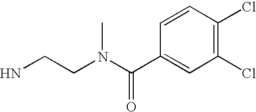 | |
| 826 | 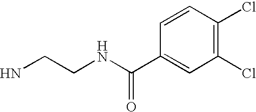 | |
| 827 | 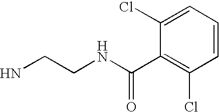 | |
| 828 | 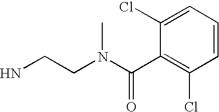 | |
| 829 | 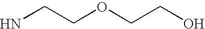 | oil, NMR(CDCl₃): 3.70(m, 6H, 3 CH₂O); 3.59(q, 2H, CH₂NH) |
| 830 | 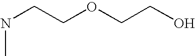 | |
| 831 | 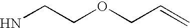 | |
| 832 | 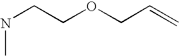 | oil, NMR(CDCl₃): 5.88(m, 1H, CH₂CH═CH₂); 5.30, 5.20(2m, 2H, CH═CH₂); |
| 833 | 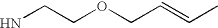 | |
| 834 | 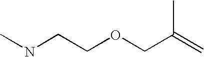 | oil, NMR(CDCl₃): 3.18, 2.92(2s, 3H, NCH₃); 1.77, 1.75(2s, 3H, butenyl-CH₃) |
| 835 | 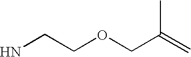 | |
| 836 | 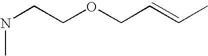 | |
| 837 | 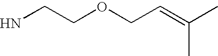 | |
| 838 | 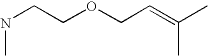 | |
| 839 | 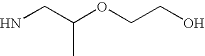 | |
| 840 | 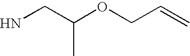 | |
| 841 | 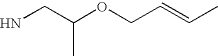 | |
| 842 | 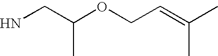 | |
| 843 | 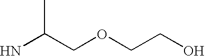 | |
| 844 | 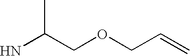 | |
| 845 | 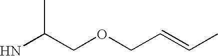 | |
| 846 | 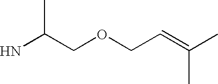 | |
| 847 | 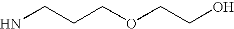 | |
| 848 | 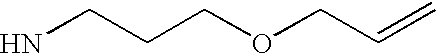 | oil, NMR(CDCl₃): 4.16(tr, 2H, CH₂O); 1.80, 1.78(2 s, 6H, 2 CH₃) |
| 849 | 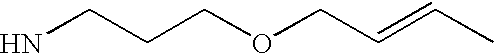 | |
| 850 | 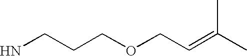 | |
| 851 | 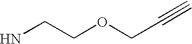 | |
| 852 | 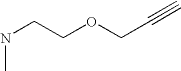 | oil, NMR(CDCl₃): 3.18, 2.91(2s, 3H, NCH₃); 2.48 (m, 1H, alkynyl- H) |
| 853 | 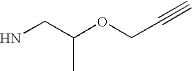 | |
| 854 | 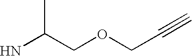 | |
| 855 | 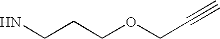 | |
| 856 | 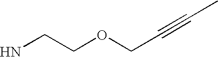 | |
| 857 | 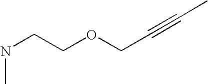 | oil, NMR(CDCl₃): 3.18, 2.91(2s, 3H, NCH₃); 1.83 (m, 3H, butynyl- CH₃) |
| 858 | 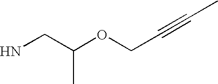 | |
| 859 | 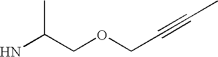 | |
| 860 | 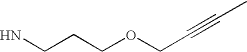 | |
| 861 | 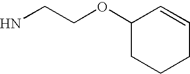 | |
| 862 | 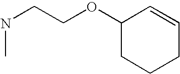 | oil, NMR(CDCl₃): 5.88, 5.71(2 m, 2H, olef. H); 3.18, 2.92(2 s, 3H, NCH₃) |
| 863 | 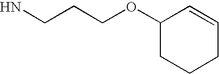 | |
| 864 | 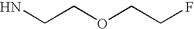 | oil, NMR(CDCl₃): 4.62, 4.48(2 tr, 2H, CH₂F); |
| 865 | 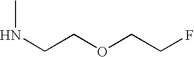 | Oil |
| 866 | 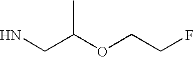 | |
| 867 | 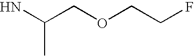 | |
| 868 | 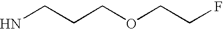 | |
| 869 | 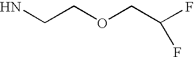 | |
| 870 | 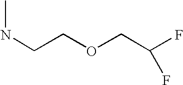 | |
| 871 | 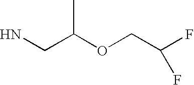 | |
| 872 | 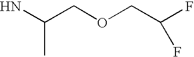 | |
| 873 | 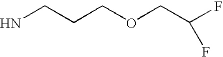 | |
| 874 | 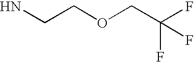 | |
| 875 | 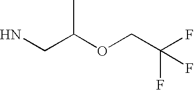 | |
| 876 | 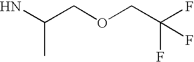 | |
| 877 | 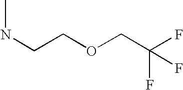 | |
| 878 | 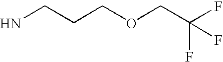 | |
| 879 | 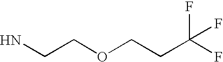 | |
| 880 | 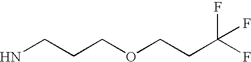 | |
| 881 | 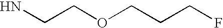 | |
| 882 | 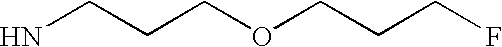 | |
| 883 | 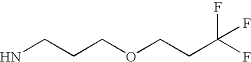 | |
| 884 | 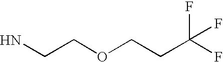 | |
| 885 | 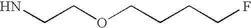 | |
| 886 | 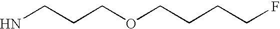 | |
| 887 | 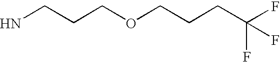 | |
| 888 | 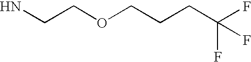 | |
| 889 | 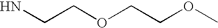 | |
| 890 | 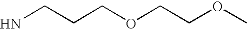 | |
| 891 | 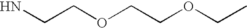 | |
| 892 | 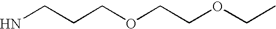 | |
| 893 | 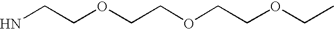 | |
| 894 | 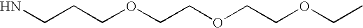 | oil, NMR(CDCl₃): 1.92(quintet, 2H; CH₂CH₂CH₂); 1.12(tr, 3H, CH₃) |
| 895 | 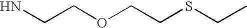 | |
| 896 | 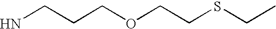 | |
| 897 | 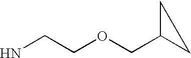 | |
| 898 | 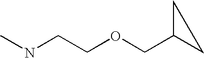 | oil, NMR(CDCl₃): 3.18, 2.92(2 3H, NCH₃); 1.06 (m, 1H, cyclopropyl-CH), 0.55, 0.21(2 m, 4H, cyclopropyl- CH₂) |
| 899 | 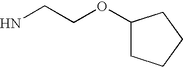 | |
| 900 | 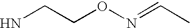 | |
| 901 | 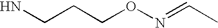 | |
| 902 | 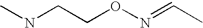 | |
| 93 | 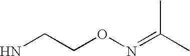 | |
| 904 | 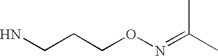 | |
| 905 | 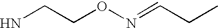 | |
| 906 | 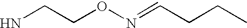 | |
| 907 | 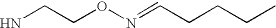 | |
| 908 | 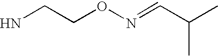 | |
| 909 | 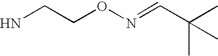 | |
| 910 | 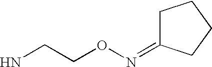 | |
| 911 | 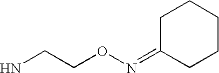 | |
| 912 | 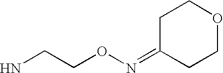 | |
| 913 | 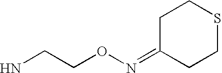 | |
| 914 | 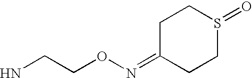 | |
| 915 | 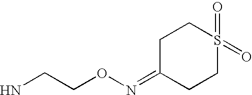 | |
| 916 | 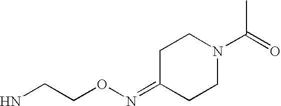 | |
| 917 | 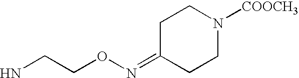 | |
| 918 | 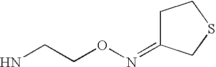 | |
| 919 | 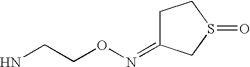 | |
| 920 | 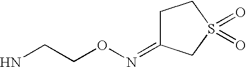 | |
| 921 | 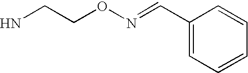 | |
| 922 | 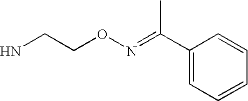 | |
| 923 | 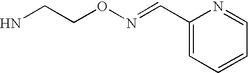 | |
| 924 | 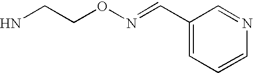 | 83 |
| 925 | 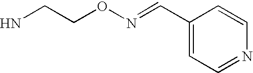 | oil, NMR(CDCl₃): 8.59, 7.40(2 d, 4H, pyridine-H); 8.0(s, 1H, oxime-H), 4.40 (tr, 2H, CH₂O) |
| 926 | 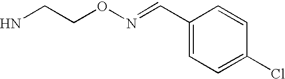 | |
| 927 | 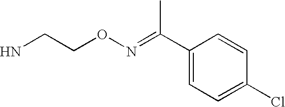 | |
| 928 | 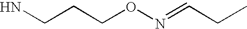 | |
| 929 | 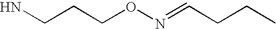 | |
| 930 | 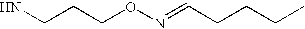 | oil, NMR(CDCl₃): 7.34(tr, 1H, oxime-H, anti form), 3.58(q, 2H, CH₂NH) |
| 931 | 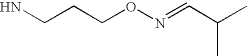 | 56-58 |
| 932 | 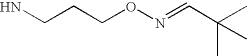 | |
| 933 | 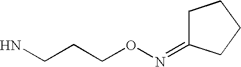 | oil, NMR(CDCl₃): 4.17(tr, 2H, CH₂O); 2.23, 1.70(2 m, 8H, cyclopentyl-H) |
| 934 | 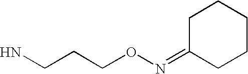 | 73-75 |

| Ex. No. | NR⁴R⁵ | m.p.[° C.] |
|---|---|---|
| 935 | 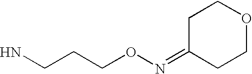 | |
| 936 | 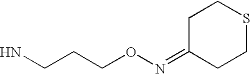 | |
| 937 | 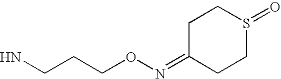 | |
| 938 | 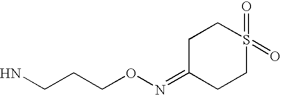 | |
| 939 | 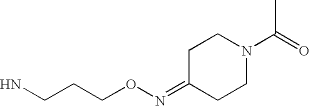 | |
| 940 | 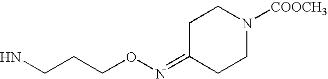 | |
| 941 | 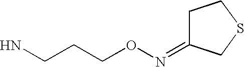 | |
| 942 | 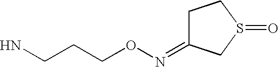 | |
| 943 | 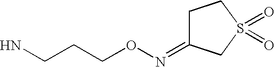 | |
| 944 | 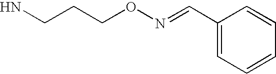 | 107-110 |
| 945 | 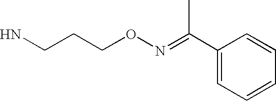 | 95-96 |
| 946 | 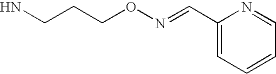 | 91-94 |
| 947 | 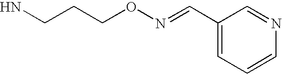 | |
| 948 | 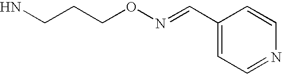 | |
| 949 | 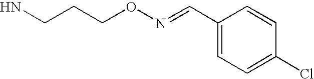 | |
| 950 | 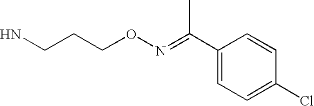 | |
| 951 | 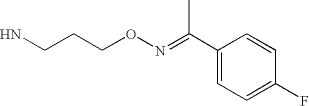 | |
| 952 | 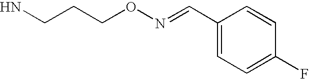 | |
| 953 | 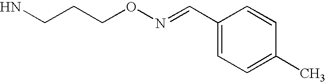 | |
| 954 | 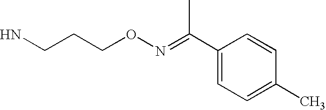 | |
| 955 | 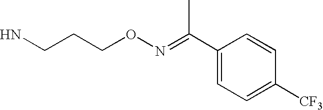 | |
| 956 | 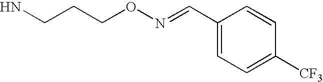 | |
| 957 | 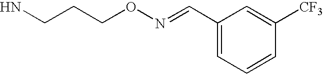 | |
| 958 | 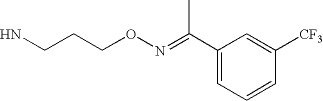 | |
| 959 | 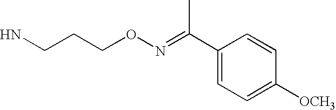 | |
| 960 | 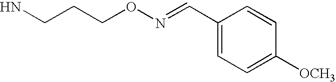 | |
| 961 | 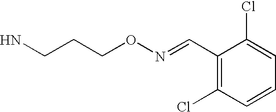 | |
| 962 | 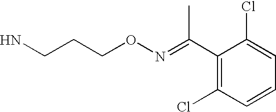 | |
| 963 | 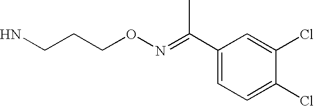 | |
| 964 | 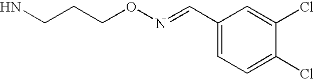 | |
| 965 | 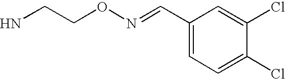 | |
| 966 | 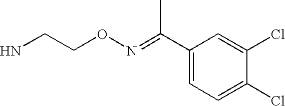 | |
| 967 | 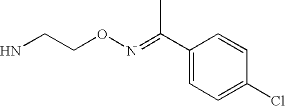 | |
| 968 | 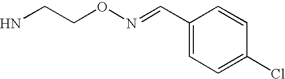 | |
| 969 | 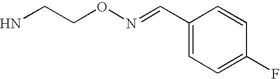 | |
| 970 | 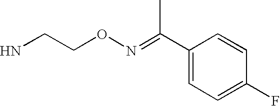 | |
| 971 | 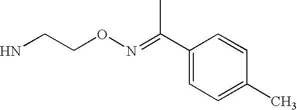 | |
| 972 | 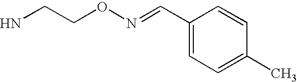 | |
| 973 | 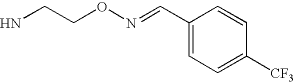 | |
| 974 | 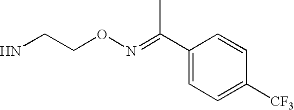 | |
| 975 | 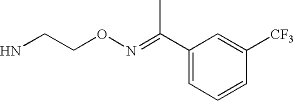 | |
| 976 | 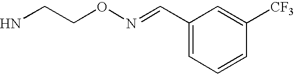 | |
| 977 | 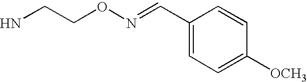 | |
| 978 | 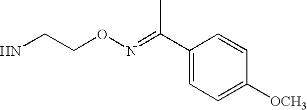 | |
| 979 | 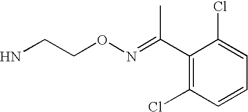 | |
| 980 | 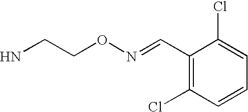 | |
| 981 | 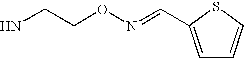 | |
| 982 | 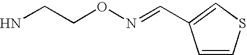 | |
| 983 | 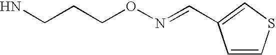 | |
| 984 | 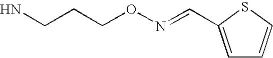 | |
| 985 | 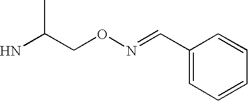 | oil |
| 986 | 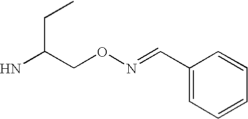 | oil |
| 987 | 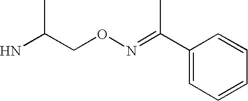 | oil |
| 988 | 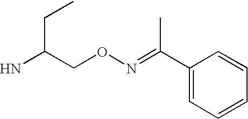 | |
| 989 | 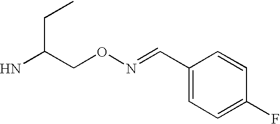 | 96-100 |
| 990 | 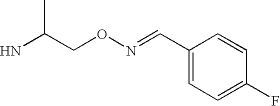 | oil |
| 991 | 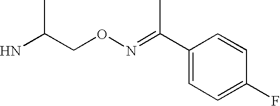 | |
| 992 | 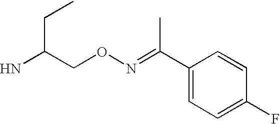 | |
| 993 | 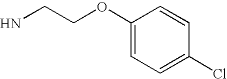 | |
| 994 | 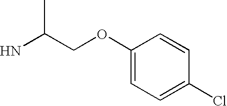 | |
| 995 | 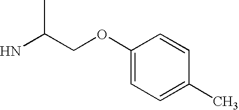 | |
| 996 | 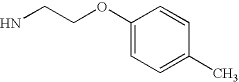 | |
| 997 | 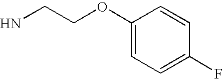 | |
| 998 | 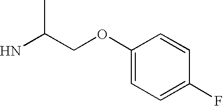 | |
| 999 | 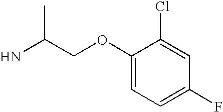 | oil |
| 1000 | 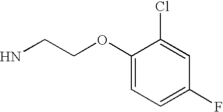 | |
| 1001 | 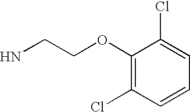 | |
| 1002 | 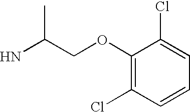 | |
| 1003 | 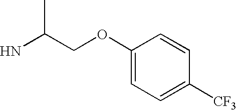 | |
| 1004 | 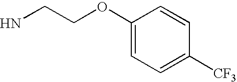 | |
| 1005 | 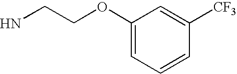 | |
| 1006 | 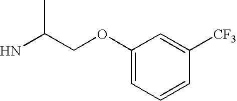 | |
| 1007 | 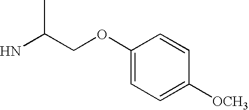 | |
| 1008 | 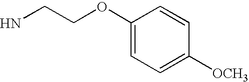 | |
| 1009 | 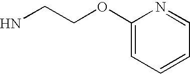 | |
| 1010 | 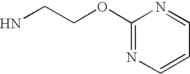 | |
| 1011 | 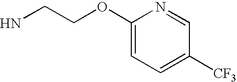 | |
| 1012 | 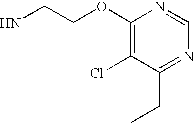 | |
| 1013 | 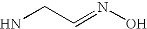 | 150 |
| 1014 | 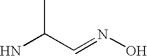 | 158-159 |
| 1015 | 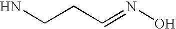 | 98 |
| 1016 | 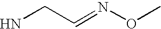 | 90-92 |
| 1017 | 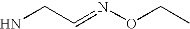 | 98-100 |
| 1018 | 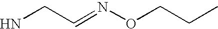 | |
| 1019 | 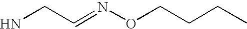 | |
| 1020 | 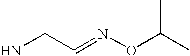 | 60-62 |
| 1021 | 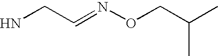 | |
| 1022 | 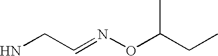 | |
| 1023 | 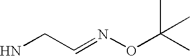 | |
| 1024 | 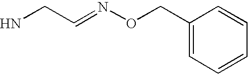 | oil, NMR(CDCl₃): 7.55(tr, 1H, oxime-H); 5.08 (2, 2H, OCH₂) |
| 1025 | 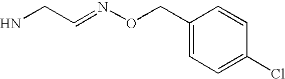 | |
| 1026 | 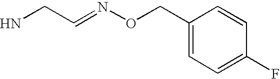 | |
| 1027 | 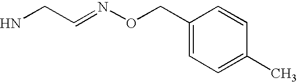 | |
| 1028 | 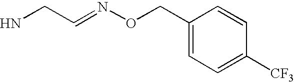 | |
| 1029 | 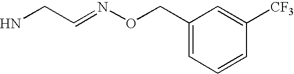 | |
| 1030 | 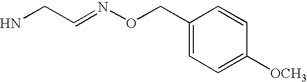 | |
| 1031 | 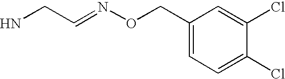 | |
| 1032 | 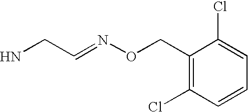 | |
| 1033 | 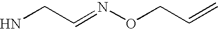 | |
| 1034 | 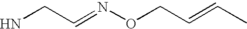 | |
| 1035 | 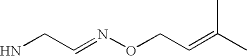 | |
| 1036 | 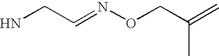 | |
| 1037 | 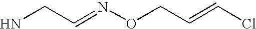 | |
| 1038 | 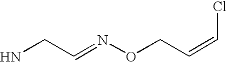 | |
| 1039 | 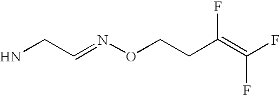 | |
| 1040 | 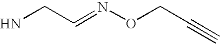 | |
| 1041 | 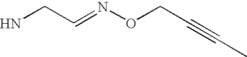 | |
| 1042 | 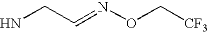 | |
| 1043 | 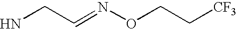 | |
| 1044 | 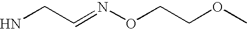 | |
| 1045 | 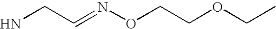 | |
| 1046 | 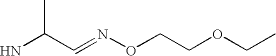 | |
| 1047 | 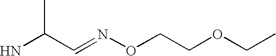 | |
| 1048 | 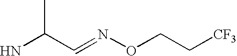 | |
| 1049 | 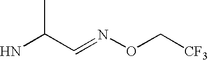 | |
| 1050 | 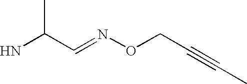 | |
| 1051 | 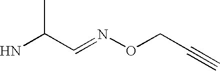 | |
| 1052 | 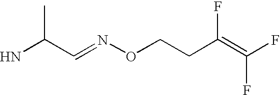 | |
| 1053 | 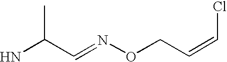 | |
| 1054 | 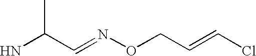 | |
| 1055 | 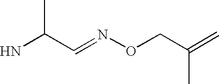 | |
| 1056 | 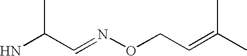 | |
| 1057 | 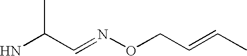 | |
| 1058 | 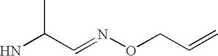 | |
| 1059 | 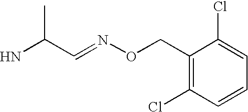 | |
| 1060 | 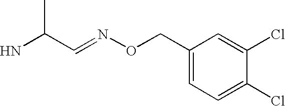 | |
| 1061 | 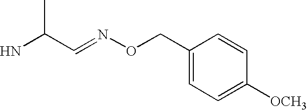 | |
| 1062 | 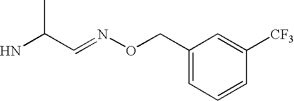 | |
| 1063 | 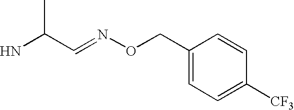 | |
| 1064 | 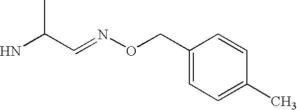 | |
| 1065 | 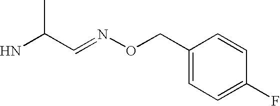 | |
| 1066 | 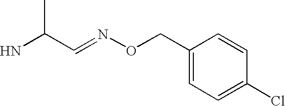 | |
| 1067 | 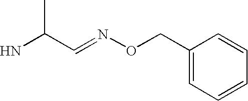 | 83-84 |
| 1068 | 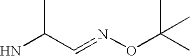 | oil, NMR(CDCl₃): 7.47(d, 1H, oxime-H); 4.87 (m, 1H, methine- H); 1.45(d, 2H, CHCH₃); 1.14(s, 9H, tert-butyl) |
| 1069 | 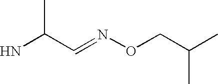 | oil, NMR(CDCl₃): oxime-H); 3.80 (d, 2H, OCH₂); 0.92(d, 6H, isobutyl-CH₃) |
| 1070 | 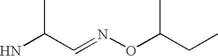 | |
| 1071 | 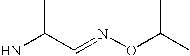 | |
| 1072 | 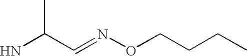 | |
| 1073 | 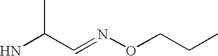 | |
| 1074 | 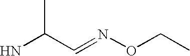 | 104-106 |
| 1075 | 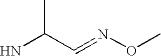 | 110-112 |
| 1076 | 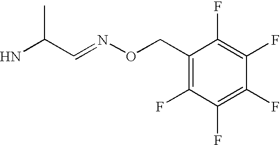 | 121-122 |
| 1077 | 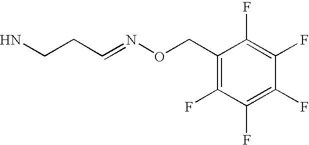 | 132-135 |
| 1078 | 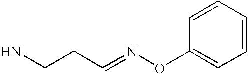 | oil |
| 1079 | 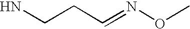 | 80 |
| 1080 | 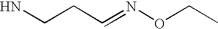 | 53 |
| 1081 | 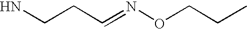 | |
| 1082 | 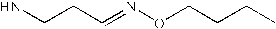 | |
| 1083 | 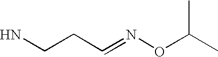 | |
| 1084 | 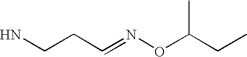 | oil |
| 1085 | 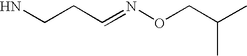 | |
| 1086 | 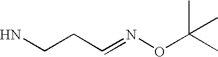 | oil, NMR(CDCl₃): 7.43(tr, 1H, oxime-H, anti); 3.71(q, 2H, CH₂NH); 1.11(s, 9H, tert-butyl) |
| 1087 | 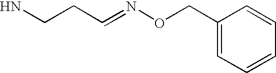 | oil, NMR(CDCl₃): 7.51(tr, 1H, oxime-H, anti); 5.0(s, 2H, CH₂O); 2.48(q, 2H, CH₂CH₂CH═) |
| 1088 | 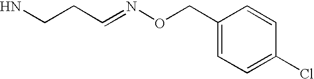 | |
| 1089 | 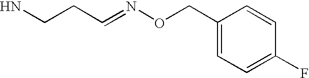 | |
| 1090 | 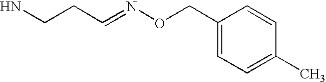 | |
| 1091 | 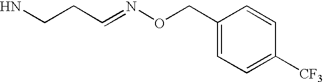 | |
| 1092 | 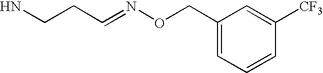 | |
| 1093 | 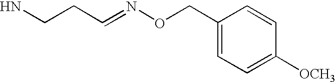 | |
| 1094 | 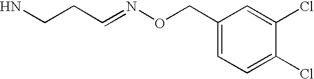 | |
| 1095 | 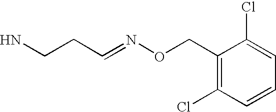 | |
| 1096 | 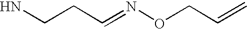 | 52-53 |
| 1097 | 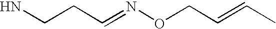 | |
| 1098 | 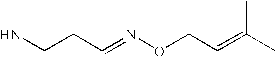 | |
| 1099 | 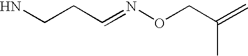 | |
| 1100 | 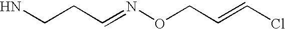 | |
| 1101 | 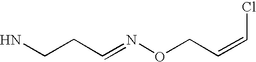 | |
| 1102 | 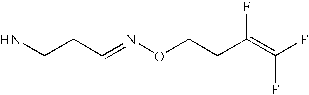 | |
| 1103 | 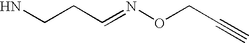 | |
| 1104 | 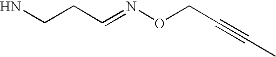 | |
| 1105 | 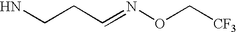 | |
| 1106 | 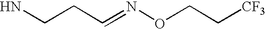 | |
| 1107 | 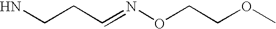 | |
| 1108 | 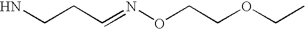 | |
| 1109 | 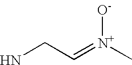 | |
| 1110 | 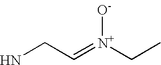 | |
| 1111 | 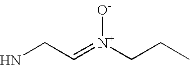 | |
| 1112 | 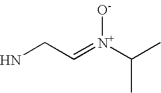 | |
| 1113 | 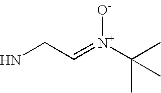 | |
| 1114 | 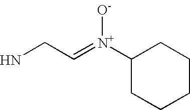 | |
| 1115 | 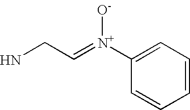 | |
| 1116 | 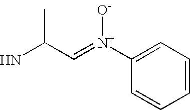 | |
| 1117 | 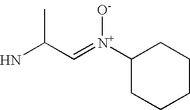 | |
| 1118 | 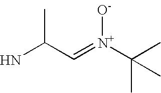 | |
| 1119 | 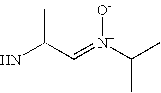 | |
| 1120 | 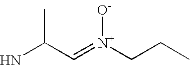 | |
| 1121 | 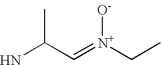 | |
| 1122 | 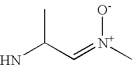 | |
| 1123 | 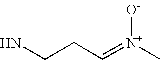 | |
| 1124 | 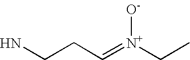 | |
| 1125 | 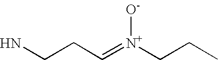 | |
| 1126 | 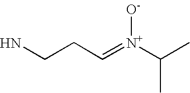 | resin, NMR (CDCl₃): 7.02(tr, 1H, nitrone-H); 4.03(quintet, 1H, isopropyl-CH); 1.35,(d, 6H, 2 CH₃) |
| 1127 | 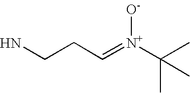 | 130-131 |
| 1128 | 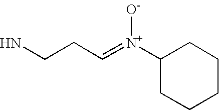 | 170-172 |
| 1129 | 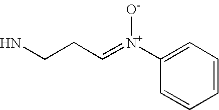 | |
| 1130 | 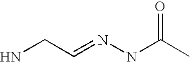 | |
| 1131 | 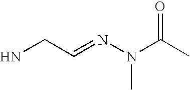 | |
| 1132 | 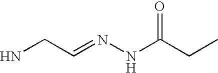 | |
| 1133 | 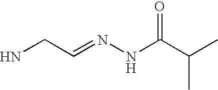 | |
| 1134 | 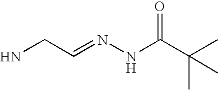 | |
| 1135 | 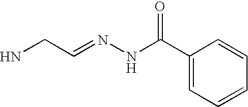 | |
| 1136 | 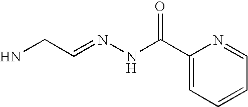 | |
| 1137 | 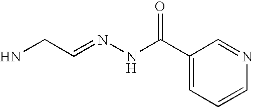 | |
| 1138 | 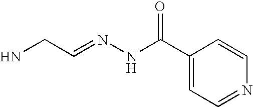 | |
| 1139 | 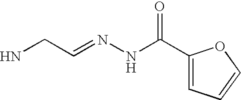 | |
| 1140 | 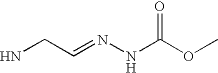 | |
| 1141 | 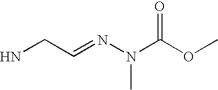 | |
| 1142 | 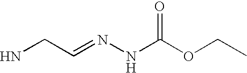 | |
| 1143 | 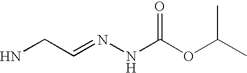 | |
| 1144 | 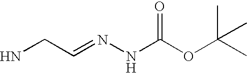 | |
| 1145 | 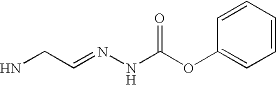 | |
| 1146 | 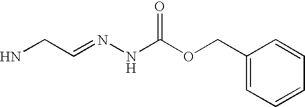 | |
| 1147 | 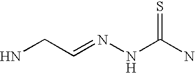 | |
| 1148 | 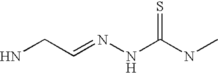 | |
| 1149 | 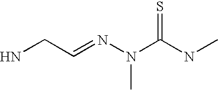 | |
| 1150 | 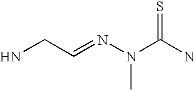 | |
| 1151 | 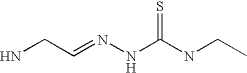 | |
| 1152 | 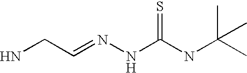 | |
| 1153 | 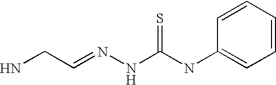 | |
| 1154 | 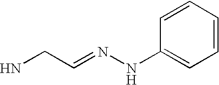 | |
| 1155 | 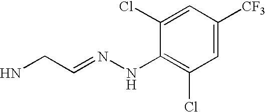 | |
| 1156 | 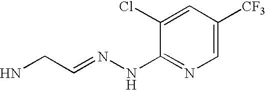 | |
| 1157 | 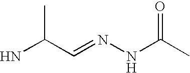 | 177-178 |
| 1158 | 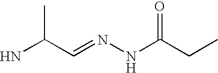 | |
| 1159 | 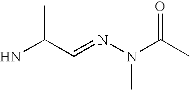 | |
| 1160 | 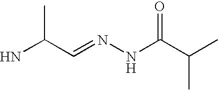 | |
| 1161 | 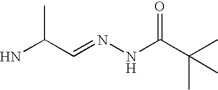 | |
| 1162 | 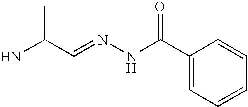 | |
| 1163 | 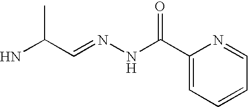 | |
| 1164 | 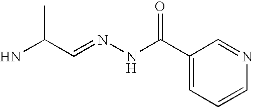 | 190 |
| 1165 | 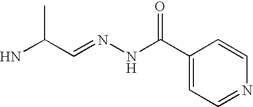 | 188 |
| 1166 | 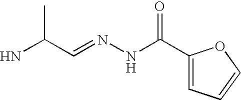 | 193-195 |
| 1167 | 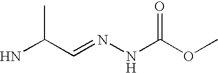 | 210-212 |

| Ex. No. | NR⁴R⁵ | m.p. [° C.] |
|---|---|---|
| 1168 | 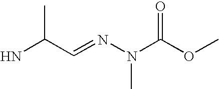 | |
| 1169 | 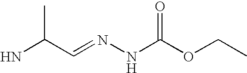 | 191 |
| 1170 | 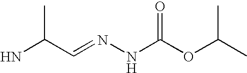 | |
| 1171 | 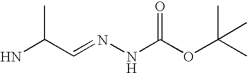 | 191 |
| 1172 | 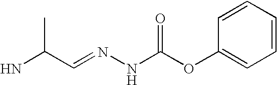 | |
| 1173 | 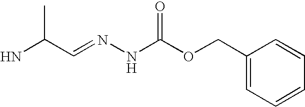 | |
| 1174 | 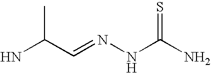 | |
| 1175 | 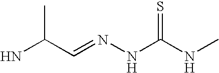 | 218 |
| 1176 | 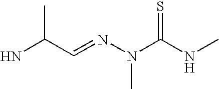 | 159 |
| 1177 | 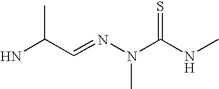 | 161 |
| 1178 | 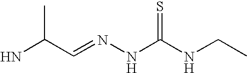 | 173 |
| 1179 | 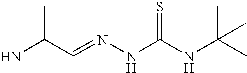 | |
| 1180 | 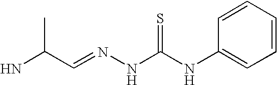 | |
| 1181 | 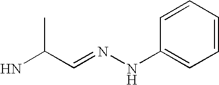 | |
| 1182 | 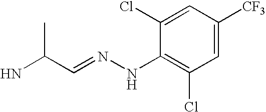 | 173-175 |
| 1183 | 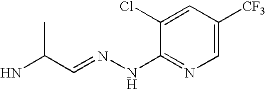 | |
| 1184 | 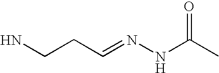 | |
| 1185 | 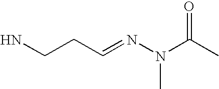 | |
| 1186 | 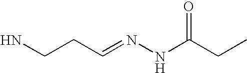 | |
| 1187 | 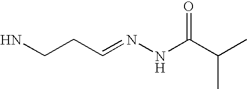 | |
| 1188 | 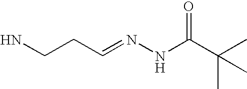 | |
| 1189 | 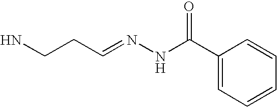 | 213 |
| 1190 | 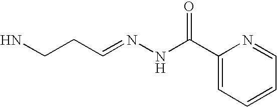 | |
| 1191 | 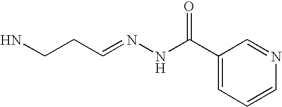 | 191-192 |
| 1192 | 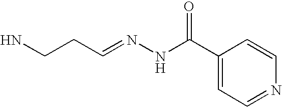 | 208 |
| 1193 | 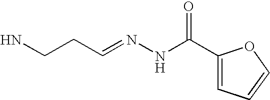 | 178-179 |
| 1194 | 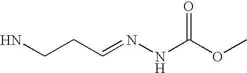 | 167-168 |
| 1195 | 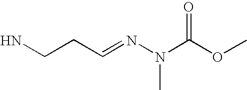 | |
| 1196 | 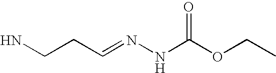 | 183-185 |
| 1197 | 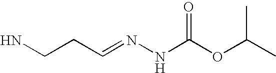 | |
| 1198 | 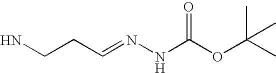 | 162 |
| 1199 | 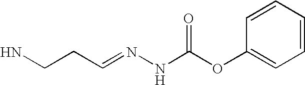 | |
| 1200 | 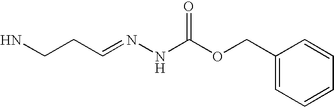 | |
| 1201 | 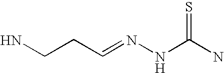 | |
| 1202 | 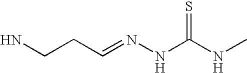 | 189-191 |
| 1203 | 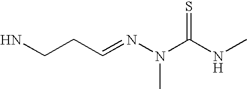 | 153-155 |
| 1204 | 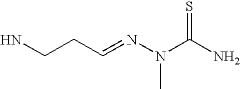 | 145 |
| 1205 | 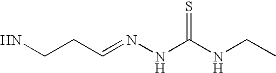 | resin, NMR(CDCl₃): 7.30(tr, 1H, hydrazone-H), 2.61(q, 2H, ethyl-CH₂), 1.22(tr, 3H, CH₃) |
| 1206 | 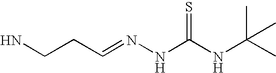 | |
| 1207 | 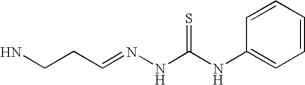 | |
| 1208 | 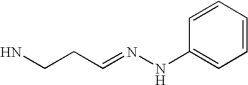 | |
| 1209 | 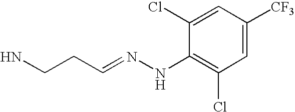 | 99-100 |
| 1210 | 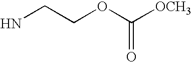 | |
| 1211 | 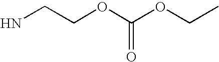 | |
| 1212 | 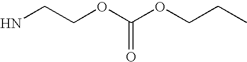 | |
| 1213 | 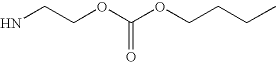 | |
| 1214 | 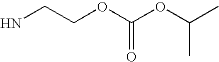 | |
| 1215 | 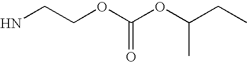 | |
| 1216 | 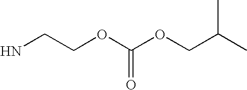 | |
| 1217 | 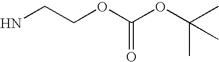 | |
| 1218 | 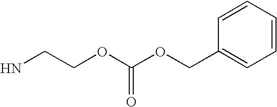 | |
| 1219 | 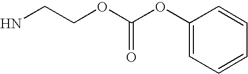 | |
| 1220 | 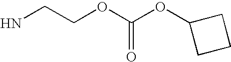 | |
| 1221 | 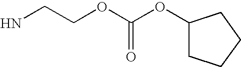 | |
| 1222 | 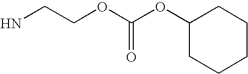 | |
| 1223 | 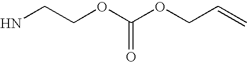 | |
| 1224 | 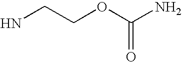 | |
| 1225 | 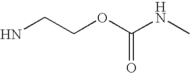 | |
| 1226 | 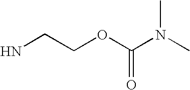 | |
| 1227 | 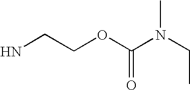 | |
| 1228 | 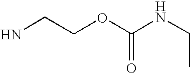 | |
| 1229 | 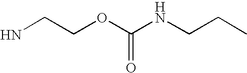 | |
| 1230 | 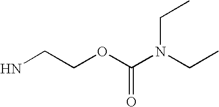 | |
| 1231 | 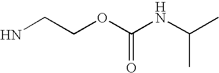 | |
| 1232 | 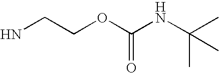 | |
| 1233 | 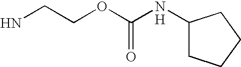 | |
| 1234 | 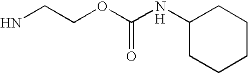 | |
| 1235 | 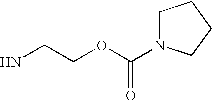 | |
| 1236 | 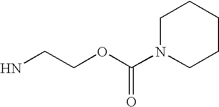 | |
| 1237 | 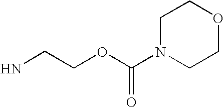 | |
| 1238 | 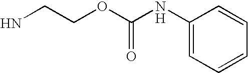 | |
| 1239 | 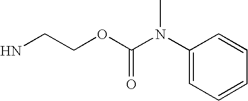 | |
| 1240 | 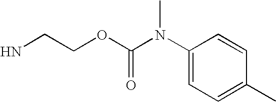 | |
| 1241 | 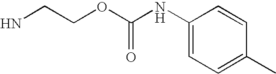 | |
| 1242 | 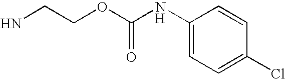 | |
| 1243 | 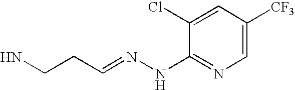 | |
| 1244 | 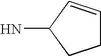 | |
| 1245 | 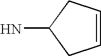 | |
| 1246 | 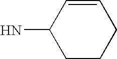 | 86-88 |
| 1247 | 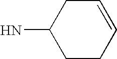 | |
| 1248 | 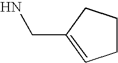 | |
| 1249 | 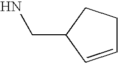 | |
| 1250 | 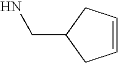 | |
| 1251 | 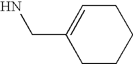 | |
| 1252 | 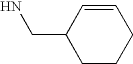 | |
| 1253 | 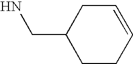 | |
| 1254 | 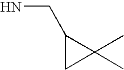 | |
| 1256 | 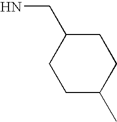 | |
| 1257 | 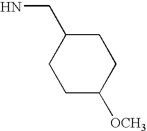 | |
| 1258 | 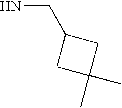 | |
| 1259 | 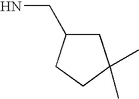 | |
| 1260 | 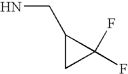 | |
| 1261 | 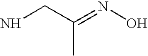 | 174-175 |
| 1262 | 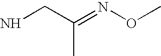 | 91-92 |
| 1263 | 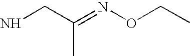 | |
| 1264 | 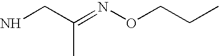 | |
| 1265 | 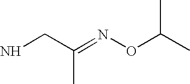 | |
| 1266 | 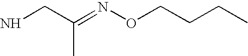 | |
| 1267 | 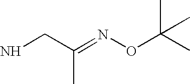 | 73-74 |
| 1268 | 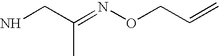 | |
| 1269 | 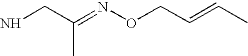 | |
| 1270 | 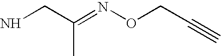 | |
| 1271 | 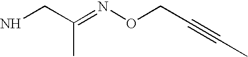 | |
| 1272 | 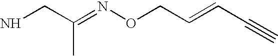 | 104 |
| 1273 | 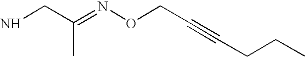 | 65 |
| 1274 | 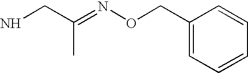 | 60-61 |
| 1275 | 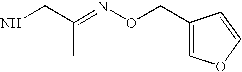 | 97-98 |
| 1276 | 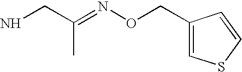 | 90-91 |
| 1277 | 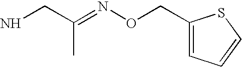 | 92-93 |
| 1278 | 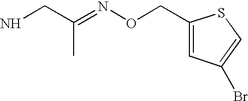 | 107-108 |
| 1279 | 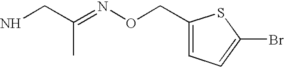 | 100-101 |
| 1280 | 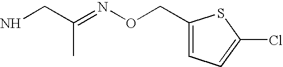 | 86-87 |
| 1281 | 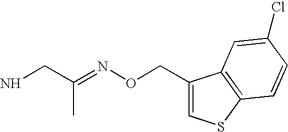 | 166-167 |
| 1282 | 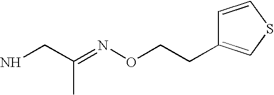 | 88 |
| 1283 | 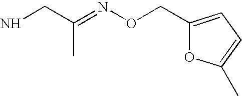 | 73-74 |
| 1284 | 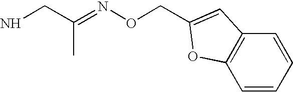 | 116-117 |
| 1285 | 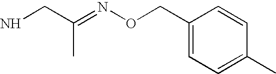 | |
| 1286 | 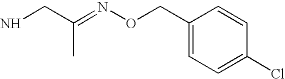 | |
| 1287 | 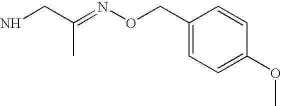 | |
| 1288 | 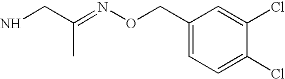 | |
| 1289 | 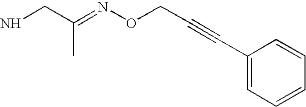 | |
| 1290 | 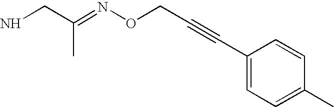 | |
| 1291 | 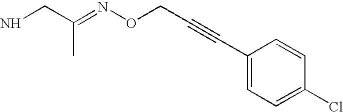 | 141-142 |
| 1292 | 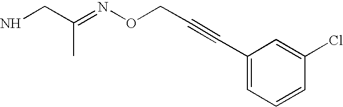 | oil, NMR: 8.84-8.94(m, 2H) 7.60(d, 1H) 7.39(m, 1H) 7.19-7.32(m, 3H) 6.62(s, NH) 4.84(s, 2H) 4.22(d, 2H) 1.98(s, 3H) |
| 1293 | 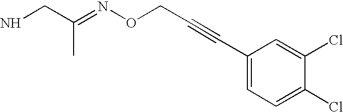 | oil, NMR: 8.86-8.92(m, 2H) 7.58(d, 1H) 7.36-7.42(m, 2H) 7.12(t, 1H) 6.66(s, NH) 4.92(s, 2H) 4.22(d, 2H) 1.97(s, 3H) |
| 1294 | 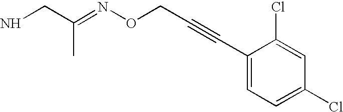 | 135-136 |
| 1295 | 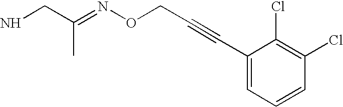 | 113-114 |
| 1296 | 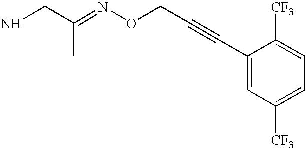 | 129 |
| 1297 | 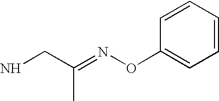 | 105-106 |
| 1298 | 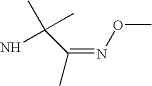 | oil, NMR: 8.82-8.88(m, 2H) 7.58(d, 1H) 7.52(s, NH) 3.80(s, 3H) 1.89(s, 3H) 1.66(s, 6H) |
| 1299 | 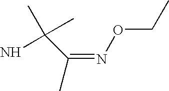 | oil, NMR: 8.80-8.88(m, 2H) 7.57(d, 1H) 7.49(s, NH) 4.02(q, 2H) 1.88(s, 3H) 1.68(s, 6H) 1.20(t, 3H) |
| 1300 | 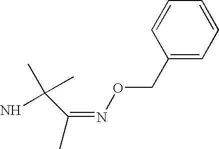 | oil, NMR: 8.84(d, 1H) 8.80(s, 1H) 7.58(d, 1H) 7.48(s, NH) 7.28-7.32(m, 5H) 5.04(s, 2H) 1.94(s, 3H) 1.66(s, 6H) |
| 1301 | 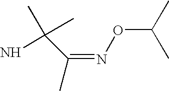 | |
| 1302 | 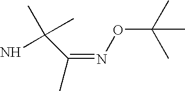 | oil, NMR: 8.81-8.88(m, 2H) 7.64(s, NH) 7.56(d, 1H) 1.86(s, 3H) 1.65(s, 6H) 1.21(s, 9H) |
| 1303 | 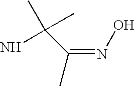 | |
| 1304 | 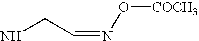 | |
| 1305 | 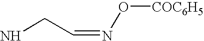 | |
| 1306 | 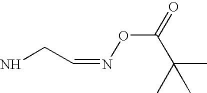 | |
| 1307 | 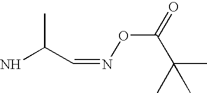 | |
| 1308 | 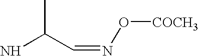 | |
| 1309 | 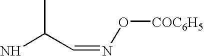 | 140 |
| 1310 | 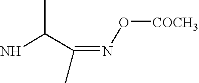 | |
| 1311 | 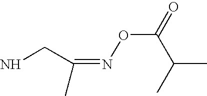 | oil, NMR: 8.88(s, 1H) 8.84(d, 1H) 7.58(d, 1H) 7.14(s, NH) 4.35(d, 2H) 2.64(sept, 1H) 2.08(s, 3H) 1.21(d, 6H) |
| 1312 | 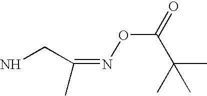 | |
| 1313 | 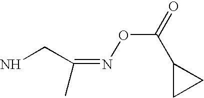 | oil, NMR: 8.86(s, 1H) 8.82(d, 1H) 7.58(d, 1H) 7.02(s, NH) 4.37(d, 1H) 2.11(s, 3H) 1.68(m, 1H) 1.08(m, 2H) 0.96(m, 2H) |
| 1314 | 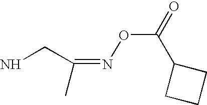 | oil, NMR: 8.88(s, 1H) 8.84(d, 1H) 7.58(d, 1H) 7.04(s, NH) 4.36(d, 2H) 3.22(quint, 1H) 2.18-2.40(m, 4H) 2.08(s, 3H) 1.92-2.06(m, 2H) |
| 1315 | 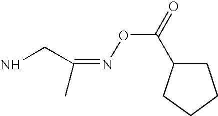 | |
| 1316 | 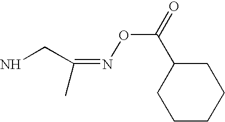 | oil, NMR: 8.85(s, 1H) 8.82(d, 1H) 7.58(d, 1H) 7.07(s, NH) 4.36(d, 2H) 2.39-2.46(m, 1H) 2.07(s, 3H) 1.92(m, 2H) 1.76(m, 2H) 1.63(m, 1H) 1.42-1.57(m, 2H) 1.22-1.38(m, 3H) |
| 1317 | 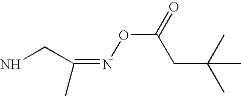 | oil, NMR: 8.86(s, 1H) 8.84(d, 1H) 7.57(d, 1H) 6.92(s, NH) 4.36(d, 2H) 2.60(s, 2H) 2.08(s, 3H) 1.07(s, 9H) |
| 1318 | 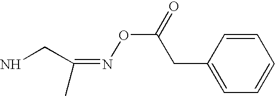 | oil, NMR: 8.84(s, 1H) 8.80(d, 1H) 7.55(d, 1H) 7.24-7.34(m, 5H) 7.16(s, NH) 4.32(d, 2H) 3.73(s, 2H) 2.01(s, 3H) |
| 1319 | 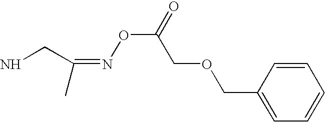 | oil, NMR: 8.86(s, 1H) 8.83(d, 1H) 7.59(d, 1H) 7.29-7.36(m, 5H) 6.88(s, NH) 4.67(s, 2H) 4.35(d, 2H) 4.24(s, 2H) 2.08(s, 3H) |
| 1320 | 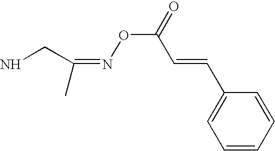 | oil, NMR: 8.90(s, 1H) 8.84(d, 1H) 7.76(d, 1H) 7.54-7.60(m, 3H) 7.39(m, 3H) 7.06(s, NH) 6.45(d, 1H) 4.41(d, 2H) 2.16(s, 3H) |
| 1321 | 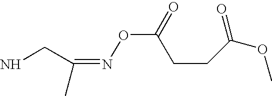 | oil, NMR: 8.87(s, 1H) 8.84(d, 1H) 7.58(d, 1H) 6.97(s, NH) 4.34(d, 2H) 3.69(s, 3H) 2.76(m, 2H) 2.70(m, 2H) 2.10(s, 3H) |
| 1322 | 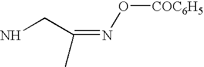 | |
| 1323 | 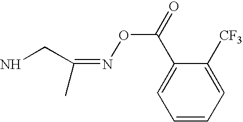 | 122-123 |
| 1324 | 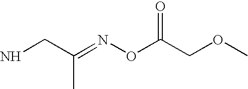 | oil, NMR: 8.87(s, 1H) 8.83(d, 1H) 7.59(d, 1H) 7.02(m, NH) 4.36(d, 2H) 4.18(s, 2H) 3.65(s, 3H) 2.09(s, 3H) |
| 1325 | 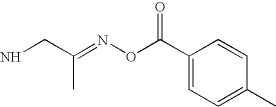 | oil, NMR: 8.94(s, 1H) 8.86(d, 1H) 7.94(d, 2H) 7.59(d, 1H) 7.27(d, 2H) 7.08(m, NH) 4.42(d, 2H) 2.42(s, 3H) 2.21(s, 3H) |
| 1326 | 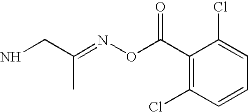 | oil, NMR: 8.85(d, 1H) 8.83(s, 1H) 7.60(d, 1H) 7.30-7.38(m, 3H) 6.94(s, NH) 4.43(d, 2H) 2.16(s, 3H) |
| 1327 | 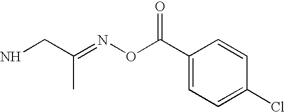 | 172-173 |
| 1328 | 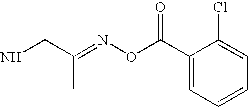 | oil, NMR: 8.91(s, 1H) 8.84(d, 1H) 7.82(m, 1H) 7.58(d, 1H) 7.43-7.50(m, 2H) 7.34-7.39(m, 1H) 7.24(s, NH) 4.42(d, 2H) 2.19(s, 3H) |
| 1329 | 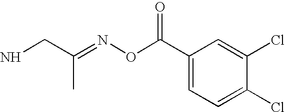 | oil, NMR: 8.92(s, 1H) 8.88(d, 1H) 8.16(d, 1H) 7.88(dd, 1H) 7.60(d, 1H) 7.57(d, 1H) 6.92(s, NH) 4.45(d, 2H) 2.24(s, 3H) |
| 1330 | 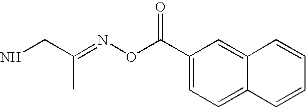 | 143 |
| 1331 | 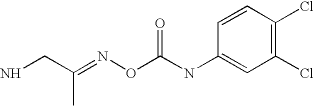 | 146-147 |
| 1332 | 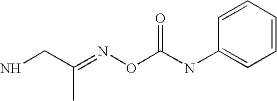 | 180-181 |
| 1333 | 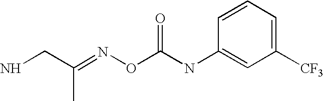 | 162-163 |
| 1334 | 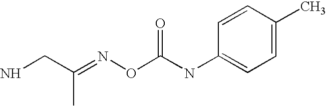 | 173-174 |
| 1335 | 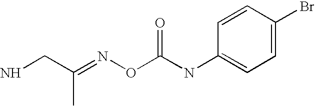 | |
| 1336 | 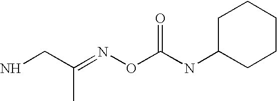 | 137-138 |
| 1337 | 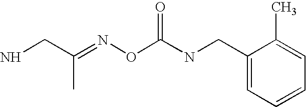 | 209-210 |
| 1338 | 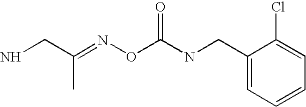 | 148-149 |
| 1339 | 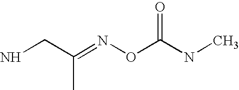 | |
| 1340 | 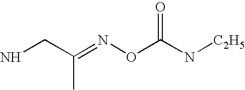 | |
| 1341 | 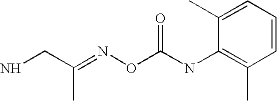 | |
| 1342 | 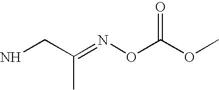 | 139-140 |
| 1343 | 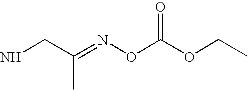 | 109-110 |
| 1344 | 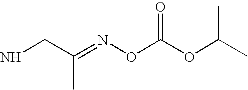 | |
| 1345 | 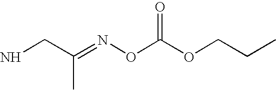 | |
| 1346 | 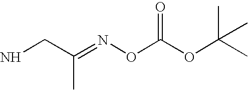 | |
| 1347 | 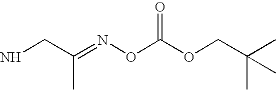 | oil, NMR: 8.88(s, 1H) 8.85(d, 1H) 7.58(d, 1H) 7.04(m, NH) 4.35(d, 2H) 3.92(s, 2H) 2.12(s, 3H) 0.96(s, 9H) |
| 1348 | 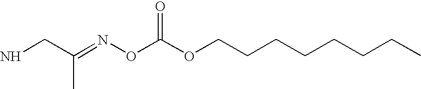 | 100-101 |
| 1349 | 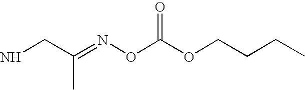 | 98-99 |
| 1350 | 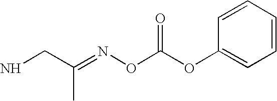 | 117-118 |
| 1351 | 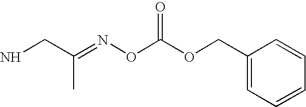 | 127-128 |
| 1352 | 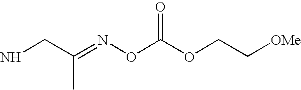 | oil, NMR: 8.88(s, 1H) 8.84(d, 1H) 7.58(d, 1H) 7.02(s, NH) 4.36-4.40(m, 4H) 3.65(m, 2H) 3.40(s, 3H) 2.10(s, 3H) |
| 1353 | 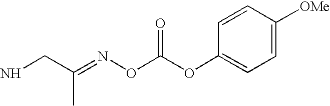 | 150 |
| 1354 | 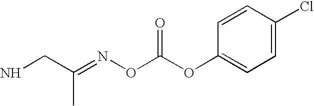 | oil, NMR: 8.94(s, 1H) 8.87(d, 1H) 7.58(d, 1H) 7.32(d, 2H) 7.08(d, 2H) 6.68(s, NH) 4.36(d, 2H) 2.14(s, 3H) |
| 1355 | 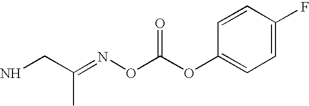 | oil, NMR: 8.91(s, 1H) 8.85(d, 1H) 7.58(d, 1H) 7.17(m, 2H) |

| Ex. No. | NR⁴R⁵ | m.p. [° C.] |
|---|---|---|
| | | 7.04 (m, 2H) |
| | | 6.70 (s, NH) |
| | | 4.38 (d, 2H) |
| | | 2.16 (s, 3H) |
| 1356 | 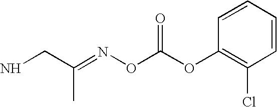 | 133-134 |
| 1357 | 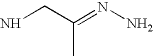 | 194-195 |
| 1358 | 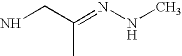 | oil, NMR: 8.90 (s, 1H) 8.85 (d, 1H) 7.59 (d, 1H) 7.06 (s, NH) 4.16 (d, 2H) 2.92 (s, 3H) 1.80 (s, 3H) |
| 1359 | 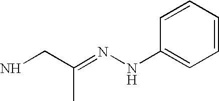 | |
| 1360 | 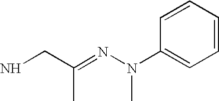 | oil, NMR: 8.88 (s, 1H) 8.83 (d, 1H) 7.58 (d, 1H) 7.38 (s, NH) 7.22-7.27 (m, 2H) 6.88-6.94 (m, 1H) 6.84 (m, 2H) 4.34 (d, 2H) 3.03 (s, 3H) 2.04 (s, 3H) |
| 1361 | 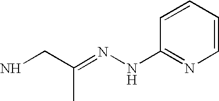 | 170 |
| 1362 | 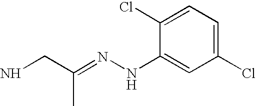 | |
| 1363 | 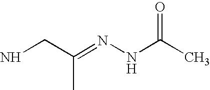 | 225-226 |
| 1364 | 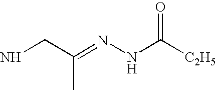 | |
| 1365 | 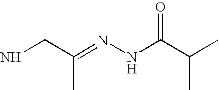 | |
| 1366 | 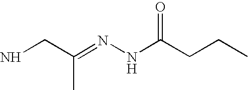 | 150-151 |
| 1367 | 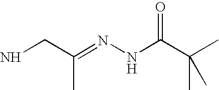 | 153-154 |
| 1368 | 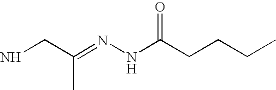 | 176-177 |
| 1369 | 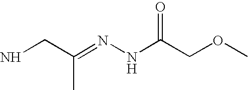 | 158-159 |
| 1370 | 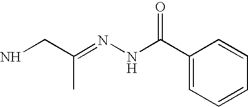 | 189 |
| 1371 | 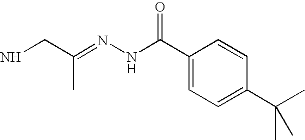 | 173-174 |
| 1372 | 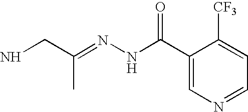 | 180-181 |
| 1373 | 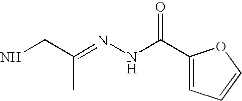 | 162-163 |
| 1374 | 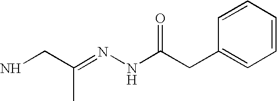 | 151-152 |
| 1375 | 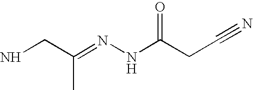 | |
| 1376 | 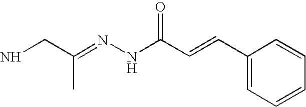 | |
| 1377 | 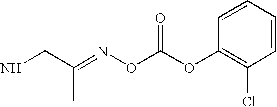 | |
| 1378 | 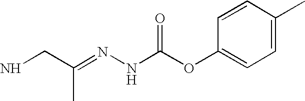 | |
| 1379 | 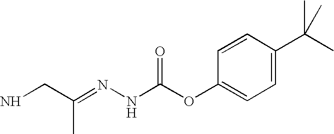 | |
| 1380 | 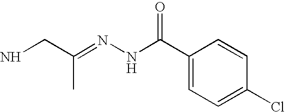 | 220 |
| 1381 | 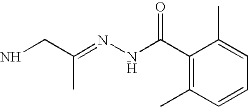 | 247-248 |
| 1382 | 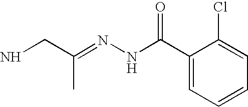 | |
| 1383 | 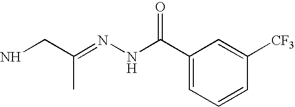 | |
| 1384 | 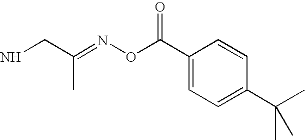 | |
| 1385 | 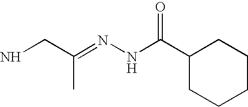 | 170-171 |
| 1386 | 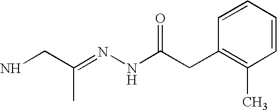 | 216-217 |
| 1387 | 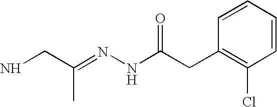 | |
| 1388 | 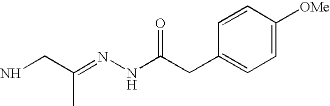 | |
| 1389 | 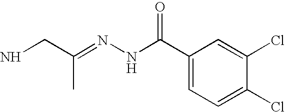 | |
| 1390 | 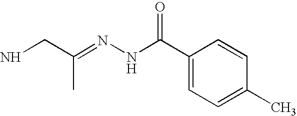 | |
| 1391 | 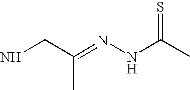 | |
| 1392 | 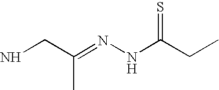 | |
| 1393 | 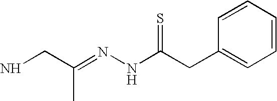 | 202-203 |
| 1394 | 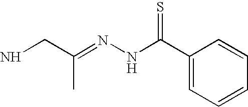 | |
| 1395 | 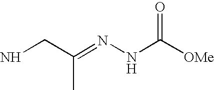 | |
| 1396 | 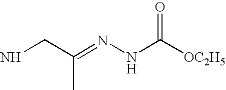 | oil, NMR: 8.90(s, 1H) 8.84 (d, 1H) 7.78 (s, NH) 7.58 (d, 1H) 7.20 (s, NH) 4.20-4.26 (m, 4H) 1.94 (s, 3H) 1.30 (t, 3H) |
| 1397 | 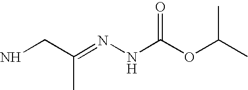 | |
| 1398 | 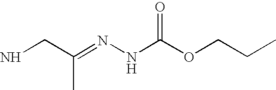 | |
| 1399 | 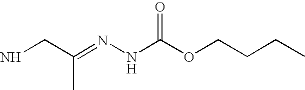 | oil, NMR: 8.91 (s, 1H) 8.84 (d, 1H) 7.76 (s, 1H) 7.58 (d, 1H) 7.22 (s, NH) 4.28 (d, 2H) 4.18 (t, 2H) 1.95 (s, 3H) 1.60-1.72 (m, 2H) 1.25-1.42 (m, 2H) 0.86-0.96 (m, 3H) |
| 1400 | 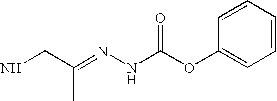 | |
| 1401 | 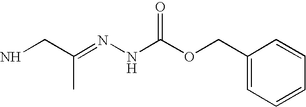 | |
| 1402 | 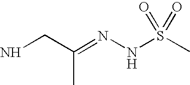 | 173-174 |
| 1403 | 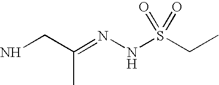 | |
| 1404 | 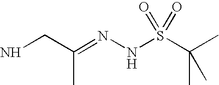 | |
| 1405 | 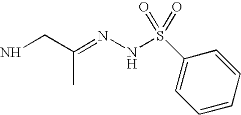 | 156-157 |
| 1406 | 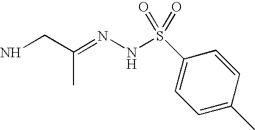 | |
| 1407 | 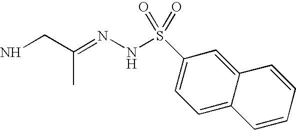 | |
| 1408 | 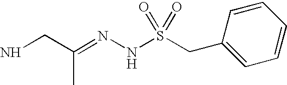 | 178-179 |
| 1409 | 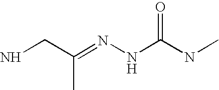 | |
| 1410 | 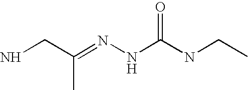 | |
| 1411 | 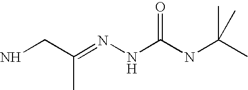 | |
| 1412 | 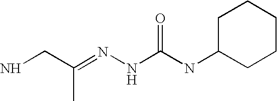 | 171 |
| 1413 | 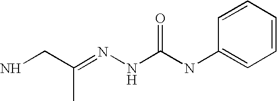 | 226-227 |
| 1414 | 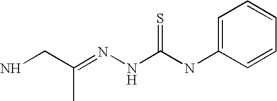 | oil, NMR: 9.08 (m, NH) 8.94 (d, 1H) 8.86 (s, 1H) 7.82 (d, 1H) 7.46-7.52 (m, 2H) 7.30-7.37 (m, 2H) 7.18 (m, 1H) 4.19 (d, 2H) 2.04 (s, 3H) |
| 1415 | 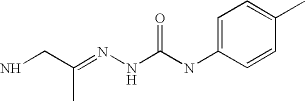 | |
| 1416 | 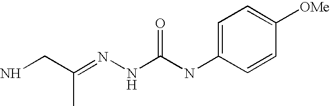 | |
| 1417 | 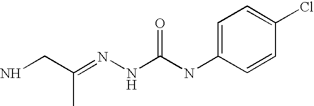 | |
| 1418 | 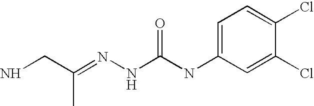 | oil, NMR: 9.88 (s, NH) 9.15 (s, 1H) 9.04 (m, NH) 8.94 (d, 1H) 8.88 (s, 1H) 7.84-7.90 (m, 2H) 7.50-7.56 (m, 1H) 7.38 (d, 1H) 4.16 (d, 2H) 1.92 (s, 3H) |
| 1419 | 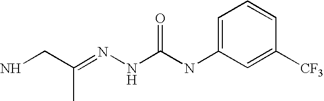 | oil, NMR: 9.84 (s, NH) 9.17 (s, 1H) 9.06 (m, NH) 8.92 (d, 1H) 8.86 (s, 1H) 7.82-7.94 (m,3H) 7.48 (t, 1H) 7.32 (d, 1H) 4.18 (d, 2H) 1.94 (s, 3H) |
| 1420 | 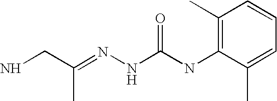 | 247-248 |
| 1421 | 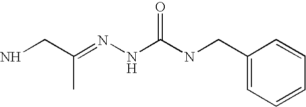 | 202-203 |
| 1422 | 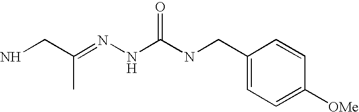 | |
| 1423 | 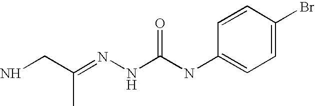 | |
| 1424 | 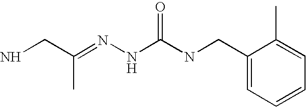 | 216-217 |
| 1425 | 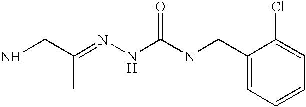 | |
| 1426 | 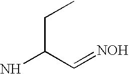 | |
| 1427 | 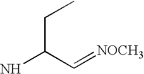 | oil, NMR: 8.84 (s, 1H) 8.82 (d, 1H) 7.58 (d, 1H) 7.44 (d, 1H) 6.45 (s, NH) 4.75 (m, 1H) 3.84 (s, 3H) 1.85 (sext, 1H) 1.70 (sext, 1H) 1.05 (t, 3H) |
| 1428 | 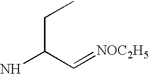 | oil, NMR: 8.87 (s, 1H) 8.84 (d, 1H) 7.59 (d, 1H) 7.46 (d, 1H) 6.44 (s, NH) 4.78 (m, 1H) 4.06 (q, 2H) 1.90 (sext, 1H) 1.74 (sext, 1H) 1.24 (t, 3H) 1.05 (t, 3H) |
| 1429 | 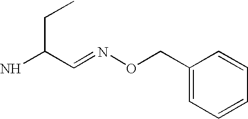 | oil, NMR: 8.84 (d, 1H) 8.81 (s, 1H) 7.58 (d, 1H) 7.44 (d, 1H) 7.29-7.36 (m, 5H) 6.42 (s, NH) 5.05 (s, 2H) 4.76 (m, 1H) 1.86 (sext, 1H) 1.66 (sext, 1H) 0.97 (t, 3H) |
| 1430 | 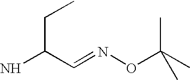 | oil, NMR: 8.86 (s, 1H) 8.84 (d, 1H) 7.60 (d, 1H) 7.44 (d, 1H) 6.49 (s, NH) 4.79 (m, 1H) 4.06 (q, 2H) 1.84 (sext, 1H) 1.70 (sext, 1H) 1.25 (s, 9H) 1.01 (t, 3H) |
| 1431 | 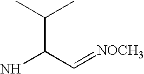 | oil, NMR: 8.86 (s, 1H) 8.84 (d, 1H) 7.59 (d, 1H) 7.46 (d, 1H) 6.47 (s, NH) 4.75 (m, 1H) 3.82 (s, 3H) 2.18 (m, 1H) 1.04 (t, 6H). |
| 1432 | 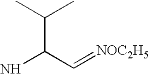 | oil, NMR: 8.87 (s, 1H) 8.83 (d, 1H) 7.59 (d, 1H) 7.48 (d, 1H) 6.48 (s, NH) 4.77 (m, 1H) 4.06 (q, 2H) 2.18 (m, 1H) 1.24 (t, 3H) 1.04 (t, 6H) |
| 1433 | 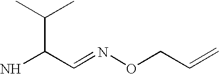 | oil, NMR: 8.86 (d, 1H) 8.84 (s, 1H) 7.60 (d, 1H) 7.54 (d, 1H) 6.46 (s, NH) 5.88 (m, 1H) 5.18-5.32 (m, 2H) 4.76 (m, 1H) 4.54 (d, 2H) 2.20 (m, 1H) 1.02 (t, 6H) |
| 1434 | 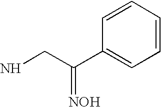 | |
| 1435 | 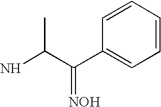 | |
| 1436 | 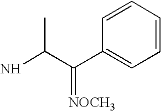 | |
| 1437 | 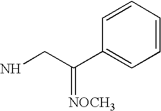 | |
| 1438 | 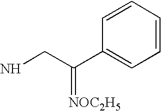 | |
| 1439 | 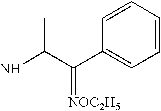 | |
| 1440 | 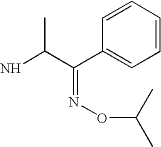 | |
| 1441 | 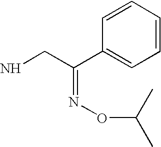 | |
| 1442 | 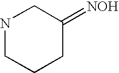 | |
| 1443 | 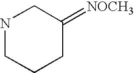 | oil, NMR: 8.86 (m, 1H) 8.64 (m, 1H) 7.59 (d, 1H) 3.84-3.90 (m, 1H) 3.76 (s, 3H) 3.20-3.31 (m, 1H) 2.62 (m, 1H) 2.44 (m, 1H) 1.82-1.95 (m, 2H) 1.63-1.80 (m, 2H) |
| 1444 | 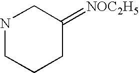 | oil, NMR: 8.84 (m, 1H) 8.62 (m, 1H) 7.58 (d, 1H) 3.97 (q, 2H) 3.78 (m, 1H) 3.20-3.31 (m, 1H) 2.62 (m, 1H) 2.43 (m, 1H) 1.84-1.96 (m, 2H) 1.62-1.81 (m, 2H) 1.18 (t, 3H) |
| 1445 | 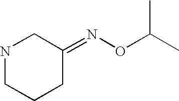 | |
| 1446 | 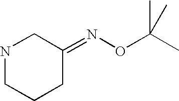 | oil, NMR: 8.84 (m, 1H) 8.62 (m, 1H) 7.59 (d, 1H) 3.92 (m, 1H) 3.76 (m, 1H) 2.62 (m, 1H) 2.42 (m, 1H) 1.82-1.94 (m, 2H) 1.60-1.81 (m, 2H) 1.21 (s, 9H) |
| 1447 | 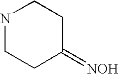 | |
| 1448 | 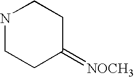 | |
| 1449 | 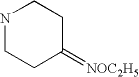 | |
| 1450 | 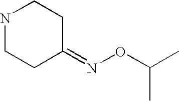 | |
| 1451 | 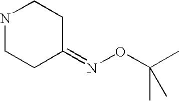 | |
| 1452 | 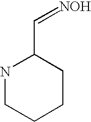 | |
| 1453 | 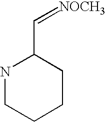 | oil, NMR: 8.82 (m, 1H) 8.66 (m, 1H) 7.56 (m, 1H) 7.29 (m, 1H) 5.58 (m, 1H) 3.92 (s, 3H) 3.18 (m, 1H) 2.08-2.22 (m, 1H) 1.66-1.83 (m, 3H) 1.52-1.62 (m, 2H) 1.38-1.50 (m, 1H) |
| 1454 | 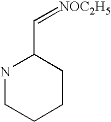 | oil, NMR: 8.80 (m, 1H) 8.64 (m, 1H) 7.59 (m, 1H) 7.35 (m, 1H) 5.60 (m, 1H) 4.08-4.20 (m, 2H) 3.09 (m, 1H) 2.02-2.24 (m, 1H) 1.64-1.86 (m, 3H) 1.55-1.63 (m, 2H) 1.40-1.52 (m, 1H) 1.22-1.28 (m, 3H) |
| 1455 | 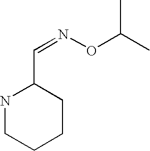 | |
| 1456 | 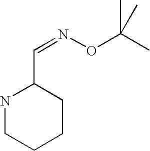 | oil, NMR: 8.82 (m, 1H) 8.66 (m, 1H) 7.58 (m, 1H) 7.30 (m, 1H) 5.62 (m, 1H) 4.10-4.20 (m, 1H) 3.09 (m, 1H) 2.04-2.24 (m, 1H) 1.63-1.84 (m, 2H) 1.52-1.62 (m, 2H) 1.38-1.50 (m, 1H) 1.32 (s, 9H) |
| 1457 | 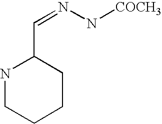 | oil, NMR: 8.77-8.92 (m, 1H) 8.66 (m, 1H) 7.84 (m, 1H) 7.58 (m, 1H) 7.12 (m, 1H) 5.56-5.65 (m, 1H) 4.08-4.26 (m, 1H) 3.04-3.32 (m, 2H) 2.30 (m, 3H) 1.88-1.96 (m, 1H) 1.66-1.82 (m, 2H) 1.50-1.64 (m, 2H) |
| 1458 | 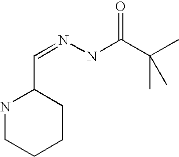 | oil, NMR: 8.78-8.92 (m, 1H) 8.68 (m, 1H) 7.82 (m, 1H) 7.58 (m, 1H) 7.09 (m, 1H) 5.57-5.64 (m, 1H) 4.07-4.28 (m, 1H) 3.04-3.30 (m, 2H) 1.88-1.96 (m, 1H) 1.66-1.82 (m, 2H) 1.50-1.64 (m, 2H) 1.26 (s, 9H) |
| 1459 | 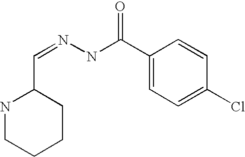 | oil, NMR: 8.79-8.88 (m, 1H) 8.72 (m, 1H) 7.78 (d, 2H) 7.58 (m, 1H) 7.30-7.42 (m, 3H) 5.58 (m, 1H) 4.16-4.68 (m, 1H) 3.08-3.24 (m, 2H) 1.90-2.05 (m, 1H) 1.64-1.87 (m, 3H) 1.40-1.62 (m, 2H) |
| 1460 | 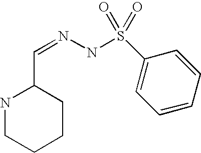 | oil, NMR: 8.82 (d, 1H) 8.64 (s, 1H) 7.88-7.96 (m, 2H) 7.46-7.60 (m, 5H) 7.18 (d, 1H) 5.50 (m, 1H) 3.99-4.50 (m, 1H) 3.04 (m, 1H) 1.98-2.22 (m, 1H) 1.60-1.80 (m, 3H) 1.30-1.58 (m, 2H) |
| 1461 | 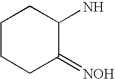 | |
| 1462 | 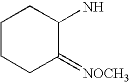 | oil, NMR: 8.88 (s, 1H) 8.82 (d, 1H) 7.58 (d, 1H) 7.06 (d, NH) 4.48 (m, 1H) 3.80 (s, 3H) 3.30-3.38 (m, 1H) 2.64-2.72 (m, 1H) 1.82-1.96 (m, 2H) 1.58-1.78 (m, 2H) 1.28-1.46 (m, 2H) |
| 1463 | 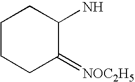 | 137-138 |
| 1464 | 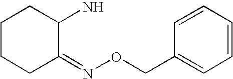 | oil, NMR: 8.86 (d, 1H) 8.82 (s, 1H) 7.59 (d, 1H) 7.27-7.36 (m, 5H) 7.04 (d, NH) 5.04 (s, 2H) 4.45 (m, 1H) 2.62-2.70 (m, 1H) 1.82-1.97 (m, 2H) 1.56-1.80 (m, 2H) 1.24-1.44 (m, 2H) |
| 1465 | 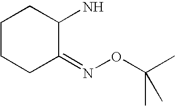 | oil, NMR: 8.90 (s, 1H) 8.86 (d, 1H) 7.58 (d, 1H) 7.12 (d, NH) 4.46 (m, 1H) 3.36-3.42 (m, 1H) 2.66-2.74 (m, 1H) 1.82-1.96 (m, 2H) 1.56-1.76 (m, 2H) 1.24-1.44 (m, 2H) 1.22 (s, 9H) |
| 1466 | 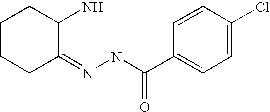 | 223-224 |
| 1467 | 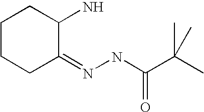 | oil, NMR: 8.90 (m, 1H) 8.82 (m, 1H) 8.42 (s, 1H) 7.57 (m, 1H) 6.95 (d, NH) 4.58 (m, 1H) 2.66-2.80 (m, 1H) 2.44-2.62 (m, 1H) 1.88-2.08 (m, 3H) 1.62-1.82 (m, 1H) 1.22-1.40 (m, 2H) 1.22 (s, 9H) |
| 1468 | 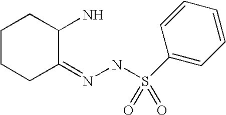 | 158-159 |
| 1469 | 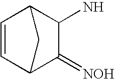 | |
| 1470 | 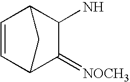 | 129-130 |
| 1471 | 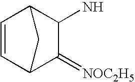 | oil, NMR: 8.82 (s, 1H) 8.78 (d, 1H) 7.58 (m, 1H) 6.28 (m, 2H) 5.90-6.07 (m, 1H) 4.86-5.07 (m, 1H) 4.04 (q, 2H) 3.97-4.04 (m, 1H) 3.40-3.58 (m, 1H) 1.92-1.98 (m, 1H) 1.70-1.75 (m, 1H) 1.22 (t, 3H) |
| 1472 | 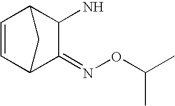 | |
| 1473 | 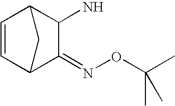 | oil, NMR: 8.84 (s, 1H) 8.80 (d, 1H) 7.58 (m, 1H) 6.26 (m, 2H) 5.90-6.06 (m, 1H) 4.86-5.04 (m, 1H) 3.95-4.02 (m, 1H) 3.44-3.60 (m, 1H) 1.92-1.98 (m, 1H) 1.70-1.75 (m, 1H) 1.24 (s, 9H) |
| 1474 | 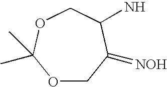 | |
| 1475 | 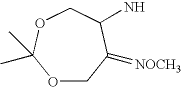 | oil, NMR: 8.84 (m, 2H) 7.58 (d, 1H) 6.99 (d, NH) 5.36 (q, 1H) 4.52 (d, 1H) 4.44 (dd, 1H) 4.32 (d, 1H) 3.86 (s, 3H) 3.42 (dd, 1H) 1.52 (s, 3H) 1.38 (s, 3H) |
| 1476 | 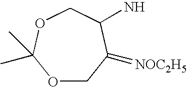 | oil, NMR: 8.84 (m, 2H) 7.60 (d, 1H) 7.04 (d, NH) 5.36 (q, 1H) 4.56 (d, 1H) 4.48 (dd, 1H) 4.34 (d, 1H) 4.06 (q, 2H) 3.42 (dd, 1H) 1.54 (s, 3H) 1.39 (s, 3H) 1.22 (t, 3H) |
| 1477 | 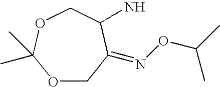 | |
| 1478 | 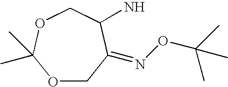 | oil, NMR: 8.86 (m, 2H) 7.59 (d, 1H) 7.06 (d, NH) 5.36 (q, 1H) 4.52 (d, 1H) 4.50 (dd, 1H) 4.32 (d, 1H) 3.42 (dd, 1H) 1.54 (s, 3H) 1.39 (s, 3H) 1.24 (s, 9H) |
| 1479 | 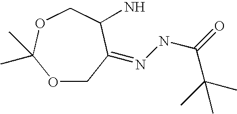 | 158 |
| 1480 | 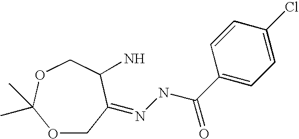 | oil, NMR: 11.58 (s, NH) 8.92 (m, 2H) 8.04 (d, 2H) 7.62 (d, 1H) 7.43 (d, 2H) 7.11 (d, NH) 5.05 (m, 1H) 4.41 (s, 2H) 4.18 (dd, 1H) 3.71 (dd, 1H) 1.42 (s, 3H) 1.39 (s, 3H) |
| 1481 | 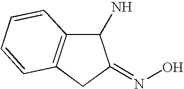 | |
| 1482 | 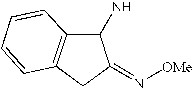 | oil, NMR: 8.95 (s, 1H) 8.86 (d, 1H) 7.58 (d, 1H) 7.52 (m, 1H) 7.27-7.36 (m, 3H) 6.22-6.35 (m, 2H) 3.97 (s, 3H) 3.86 (d, 1H) 3.62 (d, 1H) |
| 1483 | 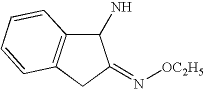 | oil, NMR: 8.94 (s, 1H) 8.84 (d, 1H) 7.60 (d, 1H) 7.52 (m, 1H) 7.28-7.35 (m, 3H) 6.22-6.36 (m, 2H) 4.18 (q, 2H) 3.88 (d, 1H) 3.63 (d, 1H) 1.28 (t, 3H) |
| 1484 | 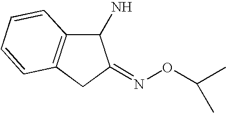 | |
| 1485 | 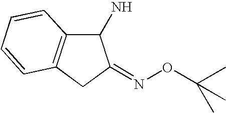 | 8.94 (s, 1H) 8.84 (d, 1H) 7.60 (d, 1H) 7.54 (m, 1H) 7.26-7.34 (m, 3H) 6.22-6.34 (m, 2H) 3.86 (d, 1H) 3.60 (d, 1H) 1.34 (s, 9H) |
| 1486 | 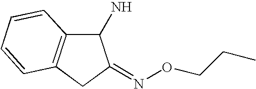 | |
| 1487 | 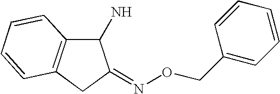 | |
| 1488 | 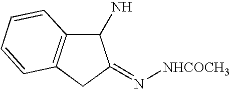 | |
| 1489 | 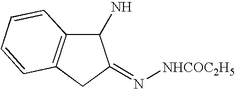 | |
| 1490 | 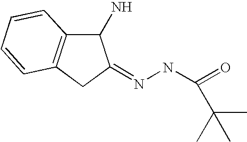 | |
| 1491 | 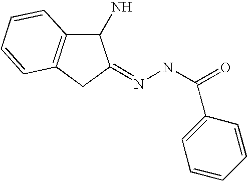 | |
| 1492 | 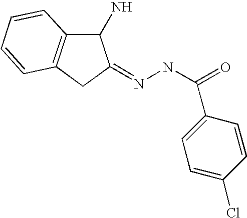 | |
| 1493 | 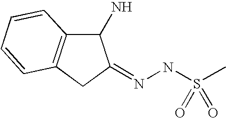 | |
| 1494 | 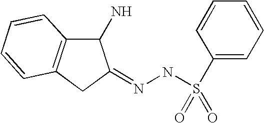 | |
| 1495 | 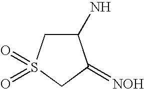 | |
| 1496 | 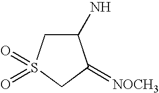 | |
| 1497 | 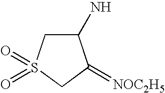 | |
| 1498 | 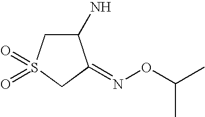 | |
| 1499 | 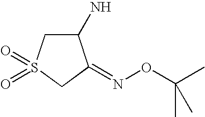 | |
| 1500 | 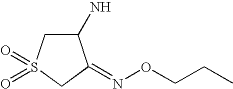 | |
| 1501 | 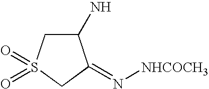 | |

### B. FORMULATION EXAMPLES

a) A dust is obtained by mixing 10 parts by weight of active compound and 90 parts by weight of talc as inert material and comminuting the mixture in a hammer mill.

b) A wettable powder which is readily dispersible in water is obtained by mixing 25 parts by weight of active compound, 65 parts by weight of kaolin-containing quartz as inert material, 10 parts by weight of potassium ligno-sulfonate and 1 part by weight of sodium oleoylmethyltaurinate as wetter and dispersant and grinding the mixture in a pinned-disk mill.

c) A dispersion concentrate which is readily dispersible in water is prepared by mixing 40 parts by weight of active compound with 7 parts by weight of a sulfosuccinic monoester, 2 parts by weight of a sodium lignosulfonate and 51 parts by weight of water and grinding the mixture in a ball mill to a fineness of below 5 microns.

d) An emulsifiable concentrate can be prepared from 15 parts by weight of active compound, 75 parts by weight of cyclohexane as solvent and 10 parts by weight of oxyethylated nonylphenol (10 EO) as emulsifier.

e) Granules can be prepared from 2 to 15 parts by weight of active compound and an inert granule carrier material such as attapulgite, pumice granules and/or quartz sand. It is expedient to use a suspension of the wettable powder of Example b) with a solids content of 30%, which is sprayed onto the surface of attapulgite granules, and these are dried and mixed intimately. The wettable powder amounts to approx. 5% by weight and the inert carrier material to approx. 95% by weight of the finished granules.

### C. BIOLOGICAL EXAMPLES

### Example 1

Germinated field bean seeds (Vicia faba) with radicles were transferred into brown glass bottles filled with tap water and subsequently populated with approximately 100 black bean aphids (Aphis fabae). Plants and aphids were then dipped for 5 seconds into an aqueous solution of the formulated compound to be examined. After the solution had run off, plant and animals were stored in a climatized chamber (16 hours of light/day, 25° C., 40-60% relative atmospheric humidity). After 3 and 6 days of storage, the effect of the compound on the aphids was determined. At a concentration of 300 ppm (based on the concentration of active compound), the compounds of Example Nos. 28a, 33, 35, 68, 77, 94, 95, 116, 120, 121, 154, 188, 190, 197, 213, 296, 299, 308, 322, 373, 393, 404, 407, 409, 409a, 414, 416, 420, 421, 426, 427, 430, 431, 438, 456, 458, 462, 491, 522, 596, 632, 633, 682, 712, 722, 723, 740, 754, 864, 865, 894, 898, 985, 986, 987, 989, 990, 1015, 1067, 1086, 1079, 1080, 1087 and 1096 caused 90-100% mortality among the aphids.

### Example 2

Germinated field bean seeds (Vicia faba) with radicles were transferred into brown glass bottles filled with tap water. Four milliliters of an aqueous solution of the formulated compound to be examined were pipetted into the brown glass bottle. The field bean was then heavily infested with about 100 black bean aphids (Aphis fabae). Plant and aphids were then stored in a climatized chamber (16 hours of light/day, 25° C., 40-60% relative atmospheric humidity). After 3 and 6 days of storage, the root-systemic effect of the compound on the aphids was determined. At a concentration of 300 ppm (based on the concentration of active compound), the compounds of Example Nos. 28a, 33, 35, 64, 68, 70, 71, 77, 79, 94, 95, 113, 114, 116, 120, 121, 154, 175, 188, 190, 191, 213, 296, 299, 308, 322, 345, 365a, 373, 393, 404, 407, 409, 409a, 414, 416, 420, 421, 426, 427, 430, 431, 438, 452, 545, 456, 458, 462, 464, 491, 522, 571, 557, 596, 632, 633, 637, 681, 682, 712, 722, 723, 829, 864, 865, 894, 898, 985, 986, 987, 989, 990, 999, 1015,1067,1086, 1079, 1080, 1087 and 1096, caused 90-100% mortality among the aphids, owing to their root-systemic action.

### Example 3

| | | |
|---|---|---|
| Solvent: | 78 | parts by weight of acetone |
| | 1.5 | parts by weight of dimethylformamide |
| Emulsifier: | 0.5 | part by weight of alkylaryl |
| | | polyglycol ether |

To produce a suitable preparation of active compound, 1 part by weight of active compound is mixed with the stated amounts of solvent and emulsifier, and the concentrate is diluted with emulsifier-containing water to the desired concentration. Disks of Chinese cabbage (Brassica pekinensis) infested by all stages of green peach aphid (Myzus persicae) were sprayed with a preparation of active compound of the desired concentration. After the desired period of time, the effect in % was determined. 100% means that all aphids have been killed; 0% means that none of the aphids has been killed.

In this test, for example, the following compounds of the Preparation Examples show good activity:

Example Nos. 1013, 1014, 1016, 1017, 1020, 1024, 1067, 1075, 1076, 1078, 1079, 1128, 1127, 1157, 1164, 1165, 1166, 1169, 1171, 1175, 1177, 1182,1191,1193, 1194, 1196, 1198, 1191, 1194, 1202, 1205, 1262, 1267, 1272, 1273, 1274, 1275, 1276, 1278, 1279, 1280, 1282, 1283, 1291, 1292, 1295, 1296, 1297, 1311, 1313, 1314, 1316, 1317, 1318, 1319, 1320, 1321, 1322, 1323, 1324, 1325, 1326, 1327, 1328, 1330, 1331, 1332, 1333, 1334, 1335,1336, 1337, 1338, 1340, 1341, 1342, 1343, 1347, 1348, 1349, 1350, 1351, 1352, 1353, 1354, 1355, 1356, 1357, 1358, 1360, 1361, 1362, 1363, 1366, 1367, 1368, 1369, 1370, 1371, 1372, 1373, 1374, 1375, 1377, 1380, 1386, 1396, 1399, 1402, 1405, 1406, 1407, 1408, 1412, 1413, 1418, 1420, 1443, 1446, 1453, 1454, 1456, 1457, 1458, 1466, 1467, 1468, 1470, 1475, 1476, 1478, 1479, 1480.

1. An amide of the formula (I) or a salt thereof 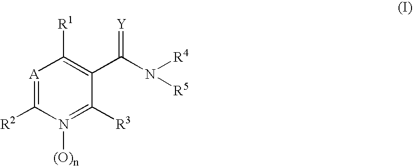
where the symbols and indices are as defined below:
A is CH or N;
Y is O or S;
n is 0 or 1;
R¹ is (C₁-C₄)-haloalkyl;
R², R³ are identical or different and are hydrogen, (C₁-C₄)-alkyl, (C₁-C₄)— haloalkyl or halogen;
R⁴ is hydrogen, (C₁-C₁₀)-alkyl, (C₃-C₁₀)-cycloalkyl, (C₃-C₁₀)-alkenyl or (C₃-C₁₀)-alkynyl, where in the alkyl, cycloalkyl, alkenyl or alkynyl groups mentioned up to three hydrogen atoms are optionally replaced by halogen, in the case of fluorine also up to the maximum number;
R⁵ is G¹SR⁶, G¹S(O)R⁷, G¹S(O)₂R⁸, G¹⁰R⁹, G¹NR¹⁰R¹¹, G²CR¹²═NOR¹³, G¹ON═CR¹⁴R¹⁵ G²CR¹²═N—NR¹⁶R¹⁷, G¹NR¹³N═CR¹⁴R¹⁵, G¹NR¹⁸NR¹⁹R²⁰, G¹ONR²¹ R²², G¹NR²³OR²⁴, G²CR¹⁰═N⁽⁺⁾(O⁽⁻⁾)R¹², R²⁵ or G¹R²⁶;
G¹ is a straight-chain or branched (C₂-C₆)-alkylene moiety, the distance between the amide nitrogen and the second radical on G¹ being at least C₂, or a (C₃-C₁₀)-cycloalkanediyl group;
G² is a straight-chain or branched (C₁-C₆)-alkylene moiety or a (C₃-C₁₀)-cycloalkanediyl group;
R⁶ is substituted (C₁-C₁₀)-alkyl, unsubstituted or substituted (C₃-C₁₀)-alkenyl, unsubstituted or substituted (C₃-C₁₀)-alkynyl, unsubstituted or substituted (C₃-C₁₀)-cycloalkyl, unsubstituted or substituted (C₄-C₁₀)-cycloalkenyl, substituted aryl or unsubstituted or substituted heterocyclyl or a radical C(NR²⁷)(NR²⁷R²⁷) in which R²⁷, R²⁷ and R²⁷ are identical or different and are in each case hydrogen, (C₁-C₁₀)-alkyl or unsubstituted or substituted aryl;
R⁷ is unsubstituted or substituted (C₁-C₁₀)-alkyl, unsubstituted or substituted (C₃-C₁₀)-alkenyl, unsubstituted or substituted (C₃-C₁₀)-alkynyl, unsubstituted or substituted (C₃-C₁₀)-cycloalkyl, unsubstituted or substituted (C₄-C₁₀)-cycloalkenyl, unsubstituted or substituted aryl or unsubstituted or substituted heterocyclyl;
R⁸ is substituted (C₁-C₁₀)-alkyl, unsubstituted or substituted (C₃-C₁₀)-alkenyl, unsubstituted or substituted (C₃-C₁₀)-alkynyl, unsubstituted or substituted (C₃-C₁₀)-cycloalkyl, unsubstituted or substituted (C₄-C₁₀)-cycloalkenyl, unsubstituted or substituted aryl, unsubstituted or substituted heterocyclyl or a group NR²⁸R²⁸, in which R²⁸ and R²⁸ are identical or different and are in each case hydrogen, unsubstituted or substituted (C₁-C₁₀)-alkyl, unsubstituted or substituted (C₃-C₁₀)-alkenyl, unsubstituted or substituted (C₃-C₁₀)-alkynyl, unsubstituted or substituted (C₃-C₁₀)-cycloalkyl, unsubstituted or substituted aryl or unsubstituted or substituted heterocyclyl and where the different radicals R²⁸ and R^{28′} are optionally linked to form a 3- to 8-membered ring which optionally contains a further heteroatom moiety;
R⁹ is substituted (C₁-C₁₀)-alkyl, unsubstituted or substituted (C₃-C₁₀)-alkenyl, unsubstituted or substituted (C₃-C₁₀)-alkynyl, unsubstituted or substituted (C₃-C₁₀)-cycloalkyl, unsubstituted or substituted (C₄-C₁₀)-cycloalkenyl, substituted aryl, unsubstituted or substituted heterocyclyl, carbamoyl, unsubstituted or substituted mono- or di-(C₁-C₁₀)-alkyl-carbamoyl, where the last-mentioned radical is optionally cyclically attached and optionally contains a heteroatom moiety, unsubstituted or substituted mono- or di-(C₃-C₁₀)-cycloalkylcarbamoyl, unsubstituted or substituted aryl- or unsubstituted or substituted N-aryl-N-(C₁-C₁₀)-alkylcarbamoyl, unsubstituted or substituted (C₁-C₁₀)-alkoxycarbonyl, unsubstituted or substituted (C₃-C₈)-cycloalkyl-(C₁-C₄)-alkoxycarbonyl, unsubstituted or substituted (C₃-C₁₀)-cycloalkoxycarbonyl, unsubstituted or substituted (C₃-C₁₀)-alkenyloxycarbonyl, unsubstituted or substituted (C₃-C₁₀)-alkynyloxycarbonyl, unsubstituted or substituted aryloxycarbonyl or unsubstituted or substituted heterocyclyloxycarbonyl;
R¹⁰ is hydrogen, unsubstituted or substituted (C₁-C₁₀)-alkyl, unsubstituted or substituted (C₃-C₁₀)-alkenyl, unsubstituted or substituted (C₃-C₁₀)-alkynyl, unsubstituted or substituted (C₃-C₁₀)-cycloalkyl, unsubstituted or substituted (C₄-C₁₀)-cycloalkenyl, unsubstituted or substituted aryl or unsubstituted or substituted heterocyclyl;
R¹¹ is substituted (C₁-C₁₀)-alkyl, unsubstituted or substituted (C₃-C₁₀)-alkenyl, unsubstituted or substituted (C₃-C₁₀)-alkynyl, substituted (C₃-C₁₀)-cycloalkyl, unsubstituted or substituted (C₄-C₁₀)-cycloalkenyl, unsubstituted or substituted aryl, unsubstituted or substituted heterocyclyl, unsubstituted or substituted (C₁-C₁₀)-alkanoyl, unsubstituted or substituted (C₃-C₁₀)-alkenoyl, unsubstituted or substituted (C₃-C₁₀)-alkynoyl, unsubstituted or substituted (C₄-C₁₀)-cycloalkanoyl, carbamoyl, unsubstituted or substituted mono- or di-(C₁-C₁₀)-alkylcarbamoyl, where the last-mentioned radical is optionally cyclically attached and optionally contains a heteroatom moiety, unsubstituted or substituted mono- or di-(C₃-C₁₀)-cycloalkylcarbamoyl, unsubstituted or substituted aryl- or unsubstituted or substituted N-aryl-N-(C₁-C₁₀)-alkylcarbamoyl, unsubstituted or substituted (C₁-C₁₀)-alkoxycarbonyl, unsubstituted or substituted (C₃-C₁₀)-alkenyloxycarbonyl, unsubstituted or substituted (C₃-C₁₀)-alkynyloxycarbonyl, unsubstituted or substituted (C₃-C₁₀)-cycloalkoxycarbonyl, unsubstituted or substituted aryloxycarbonyl, unsubstituted or substituted heterocyclyloxycarbonyl, unsubstituted or substituted (C₁-C₁₀)-alkylsulfonyl, unsubstituted or substituted arylsulfonyl, unsubstituted or substituted heterocyclylsulfonyl, unsubstituted benzoyl or benzoyl which is mono- or polysubstituted in the 2-, 4- and/or 6-positions or unsubstituted or substituted naphthoyl;
R¹² has the meanings given above for R¹⁰ or the meaning C₂-alkenyl or C₂-alkynyl; where, if R⁴ and R¹² have the meaning (C₁-C₁₀)-alkyl, these two radicals are optionally linked to form a five- to eight-membered ring system; and where furthermore, if R¹² has the meaning (C₁-C₁₀)-alkyl, the alkylene moiety G₂ and R¹² are optionally linked to form a four- to eight-membered ring system which, if chemically possible, optionally contains, in addition to carbon atoms, a heteroatom moiety, in the case of oxygen also two not directly adjacent oxygen atoms, and which is optionally also benzo-fused;
R¹³ is hydrogen, unsubstituted or substituted (C₁-C₁₀)-alkyl, unsubstituted or substituted (C₃-C₁₀)-alkenyl, unsubstituted or substituted (C₃-C₁₀)-alkynyl, unsubstituted or substituted (C₃-C₁₀)-cycloalkyl, unsubstituted or substituted (C₄-C₁₀)-cycloalkenyl, unsubstituted or substituted aryl, unsubstituted or substituted heterocyclyl, unsubstituted or substituted (C₁-C₁₀)-alkanoyl, unsubstituted or substituted (C₃-C₁₀)-alkenoyl, unsubstituted or substituted (C₃-C₁₀)-alkynoyl, unsubstituted or substituted (C₄-C₁₀)-cycloalkanoyl, unsubstituted or substituted aroyl, unsubstituted or substituted heterocyclylcarbonyl, carbamoyl, unsubstituted or substituted mono- or di-(C₁-C₁₀)-alkylcarbamoyl, where the last-mentioned radical is optionally cyclically attached and optionally contains a heteroatom moiety, unsubstituted or substituted mono- or di-(C₃-C₁₀)-cycloalkylcarbamoyl, unsubstituted or substituted aryl- or unsubstituted or substituted N-aryl-N-(C₁-C₁₀)-alkylcarbamoyl, unsubstituted or substituted (C₁-C₁₀)-alkoxycarbonyl, unsubstituted or substituted (C₃-C₁₀)-alkenyloxycarbonyl, unsubstituted or substituted (C₃-C₁₀)-alkynyloxycarbonyl, unsubstituted or substituted (C₃-C₁₀)-cycloalkoxycarbonyl, unsubstituted or substituted aryloxycarbonyl, unsubstituted or substituted heterocyclyloxycarbonyl, unsubstituted or substituted (C₁-C₁₀)-alkylsulfonyl, unsubstituted or substituted arylsulfonyl, unsubstituted or substituted heterocyclylsulfonyl, unsubstituted or substituted aroyl or unsubstituted or substituted heterocyclylcarbonyl;
R¹⁴ and R¹⁵ are identical or different, each having the meaning of R¹⁰, or R¹⁴ and R¹⁵ are linked to form a 3- to 8-membered ring which, in addition to carbon atoms, optionally contains a heteroatom moiety, if appropriate incorporating an unsubstituted or substituted benzene ring;
R¹⁶ and R¹⁷ are identical or different, each having the meaning of R¹⁰, or they are linked to form a 3- to 8-membered ring which, in addition to carbon atoms, optionally contains a heteroatom unit, or one of the radicals R¹⁶ and R¹⁷ is unsubstituted or substituted (C₁-C₁₀)-alkanoyl, unsubstituted or substituted (C₃-C₁₀)-alkenoyl, unsubstituted or substituted (C₃-C₁₀)-alkynoyl, unsubstituted or substituted (C₄-C₁₀)-cycloalkanoyl, unsubstituted or substituted aroyl, unsubstituted or substituted heterocyclylcarbonyl, carbamoyl, unsubstituted or substituted (C₁-C₁₀)-mono- or di-(C₁-C₁₀)-alkylcarbamoyl, where the last-mentioned radical is optionally cyclically attached and optionally contains a heteroatom moiety, unsubstituted or substituted mono- or di(C₃-C₁₀)-cycloalkylcarbamoyl, unsubstituted or substituted aryl- or unsubstituted or substituted N-aryl-N-(C₁-C₁₀)-alkylcarbamoyl, unsubstituted or substituted (C₁-C₁₀)-alkoxycarbonyl, unsubstituted or substituted (C₃-C₁₀)-alkenyloxycarbonyl, unsubstituted or substituted (C₃-C₁₀)-alkynyloxycarbonyl, unsubstituted or substituted (C₃-C₁₀)-cycloalkoxycarbonyl, unsubstituted or substituted aryloxycarbonyl, unsubstituted or substituted heterocyclyloxycarbonyl, the thiocarbonyl analogs of the abovementioned carbonyl derivatives, unsubstituted or substituted (C₁-C₁₀)-alkylsulfonyl, unsubstituted or substituted arylsulfonyl or unsubstituted or substituted heterocyclylsulfonyl;
R¹⁸, R¹⁹, R²⁰ are identical or different, each having the meaning of R¹⁰, or are linked together to form a 3- to 8-membered ring, optionally contains contain a heteroatom unit, or R¹⁸ and/or R¹⁹ are/is unsubstituted or substituted (C₁-C₁₀)-alkanoyl, unsubstituted or substituted (C₃-C₁₀)-alkenoyl, unsubstituted or substituted (C₃-C₁₀)-alkynoyl, unsubstituted or substituted (C₄-C₁₀)-cycloalkanoyl, unsubstituted or substituted aroyl, unsubstituted or substituted heterocyclylcarbonyl, carbamoyl, unsubstituted or substituted mono- or di-(C₁-C₁₀)-alkylcarbamoyl, where the last-mentioned radical is optionally also be cyclically attached and optionally contains a heteroatom unit, unsubstituted or substituted mono- or di-(C₃-C₁₀)-cycloalkylcarbamoyl, unsubstituted or substituted aryl- or unsubstituted or substituted N-aryl-N-(C₁-C₁₀)-alkylcarbamoyl, unsubstituted or substituted (C₁-C₁₀)-alkoxycarbonyl, unsubstituted or substituted (C₃-C₁₀)-alkenyloxycarbonyl, unsubstituted or substituted (C₃-C₁₀)-alkynyloxycarbonyl, unsubstituted or substituted (C₃-C₁₀)-cycloalkoxycarbonyl, unsubstituted or substituted aryloxycarbonyl, unsubstituted or substituted heterocyclyloxycarbonyl, the thiocarbonyl analogs of the abovementioned carbonyl derivatives, unsubstituted or substituted (C₁-C₁₀)-alkylsulfonyl, unsubstituted or substituted arylsulfonyl, unsubstituted or substituted heterocyclylsulfonyl;
R²¹ and R²² are identical or different, each having the meaning of R¹⁰, or they are linked together to form a 3- to 8-membered ring, which may contain optionally contains a heteroatom moiety, or one of the radicals R²¹ or R²² is unsubstituted or substituted (C₁-C₁₀)-alkanoyl, unsubstituted or substituted (C₃-C₁₀)-alkenoyl, unsubstituted or substituted (C₃-C₁₀)-alkynoyl, unsubstituted or substituted (C₄-C₁₀)-cycloalkanoyl, unsubstituted or substituted aroyl, unsubstituted or substituted heterocyclylcarbonyl, carbamoyl, unsubstituted or substituted mono- or di-(C₁-C₁₀)-alkylcarbamoyl, where the last-mentioned radical is optionally cyclically attached and optionally contains a heteroatom moiety, unsubstituted or substituted mono- or di-(C₃-C₁₀)-cycloalkylcarbamoyl, unsubstituted or substituted aryl- or unsubstituted or substituted N-aryl-N-(C₁-C₁₀)-alkylcarbamoyl, unsubstituted or substituted (C₁-C₁₀)-alkoxycarbonyl, unsubstituted or substituted (C₃-C₁₀)-alkenyloxycarbonyl, unsubstituted or substituted (C₃-C₁₀)-alkynyloxycarbonyl, unsubstituted or substituted (C₃-C₁₀)-cycloalkoxycarbonyl, unsubstituted or substituted aryloxycarbonyl, unsubstituted or substituted heterocyclyloxycarbonyl, unsubstituted or substituted (C₁-C₁₀)-alkylsulfonyl, unsubstituted or substituted arylsulfonyl, unsubstituted or substituted heterocyclylsulfonyl, unsubstituted or substituted aroyl or unsubstituted or substituted heterocyclylcarbonyl;
R²³ and R²⁴ are identical or different, each having the meaning of R¹³, or they are linked together to form a 3- to 8-membered ring;
R²⁵ is unsubstituted or substituted (C₄-C₁₀)-cycloalkenyl, where in this cycloalkenyl group a CH₂ unit is optionally replaced by a group C═NOR¹³ or a group C═NNR¹⁶R¹⁷;
R²⁶ is unsubstituted or substituted (C₃-C₁₀)-cycloalkenyl or substituted (C₃-C₁₀)-cycloalkyl.

2. An amide of the formula (I) as claimed in claim 1, where the symbols and indices in formula (I) are as defined below:
A is CH;
y is O;
n is 0;
R¹ is (C₁-C₄)-alkyl which is mono- or polysubstituted by F and/or Cl;
R², R³ are hydrogen;
R⁴ is hydrogen or methyl;
R⁵ is G¹SR⁶, G¹S(O)R⁷, G¹S(O)₂R⁸, G¹OR⁹, G¹NR¹⁰R¹¹, G²CR¹²═NOR¹³, G¹ON═CR¹⁴R¹⁵, G²CR¹²═NR¹⁶R¹⁷ or G²CR¹⁰═N⁽⁺⁾(O⁽⁻⁾)R¹²;
G¹ is a straight-chain or branched (C₂-C₄)-alkylene unit, the distance between the amide nitrogen and the second radical on G¹ being at least C₂, and
G² is a straight-chain or branched (C₁-C₄)-alkylene unit.

3. An amide of the formula (I) as claimed in claim 1, where R¹ is CF₃.

4. An amide of the formula (I) as claimed in claim 1, where
G¹ is CH₂—CH₂, CH₂—CH₂—CH₂, CH(CH₃)—CH₂ or CH(C₂H₅)—CH₂ and
G² is one of the groups defined above for G¹, CH₂ or CH(CH₃).

5. An amide of the formula (I) as claimed in claim 1, where substituents which can be present on the groups listed under radicals R⁶ to R²⁶ are selected from the group consisting of:
halogen, cyano, nitro, hydroxyl, thio, amino, (C₁-C₁₀)-alkanoyl, (C₃-C₁₀)-alkenoyl, (C₃-C₁₀)-alkynoyl, (C₄-C₁₀)-cycloalkanoyl, (C₁-C₁₀)-alkoxy, (C₃-C₁₀)-alkenyloxy, (C₃-C₁₀)-alkynyloxy, (C₃-C₁₀)-cycloalkoxy, (C₄-C₁₀)-cycloalkenyloxy, (C₃-C₁₀)-cycloalkyl-(C₁-C₄)-alkoxy, (C₄-C₁₀)-cycloalkenyl-(C₁-C₄)-alkoxy, (C₃-C₁₀)-cycloalkyl-(C₃-C₄)-alkenyloxy, (C₄-C₁₀)-cycloalkenyl-(C₃-C₄)-alkenyloxy, (C₁-C₄)-alkyl-(C₃-C₁₀)-cycloalkoxy, (C₂-C₄)-alkenyl-(C₃-C₁₀)-cycloalkoxy, (C₂-C₄)-alkynyl-(C₃-C₁₀)-cycloalkoxy, (C₁-C₄)-alkyl-(C₄-C₁₀)-cycloalkenyloxy, (C₂-C₄)-alkenyl-(C₄-C₁₀)-cycloalkenyloxy, (C₁-C₄)-alkoxy-(C₁-C₄)-alkoxy, (C₁-C₄)-alkoxy-(C₃-C₄)-alkenyloxy, carbamoyl, mono- and di-(C₁-C₁₀)-alkylcarbamoyl, where the last-mentioned radical is optionally cyclically attached and optionally contains a heteroatom moiety which is oxygen, sulfur, S(O) or S(O)₂, mono- and di-(C₃-C₁₀)-cycloalkylcarbamoyl, (C₁-C₁₀)-alkoxycarbonyl, (C₃-C₁₀)-cycloalkoxycarbonyl, (C₁-C₁₀)-alkanoyloxy, (C₄-C₁₀)-cycloalkanoyloxy, (C₁-C₁₀)-alkanoylamino, (C₃-C₁₀)-alkenoylamino, (C₄-C₁₀)-cycloalkanoylamino, (C₃-C₁₀)-cycloalkyl-(C₁-C₄)-alkanoylamino, the N-(C₁-C₄)-alkylamino analogs of the four last-mentioned radicals, (C₁-C₁₀)-alkylthio, (C₃-C₁₀)-alkenylthio, (C₃-C₁₀)-alkynylthio, (C₃-C₁₀)-cycloalkylthio, (C₄-C₁₀)-cycloalkenylthio, (C₃-C₁₀)-cycloalkyl-(C₁-C₄)-alkylthio, (C₄-C₁₀)-cycloalkenyl-(C₁-C₄)-alkylthio, (C₃-C₁₀)-cycloalkyl-(C₃-C₄)-alkenylthio, (C₄-C₁₀)-cycloalkenyl-(C₃-C₄)-alkenylthio, (C₁-C₄)-alkyl-(C₃-C₁₀)-cycloalkylthio, (C₂-C₄)-alkenyl-(C₃-C₁₀)-cycloalkylthio, (C₂-C₄)-alkynyl-(C₃-C₁₀)-cycloalkylthio, (C₁-C₄)-alkyl-(C₄-C₁₀)-cycloalkenylthio, (C₂-C₄)-alkenyl-(C₄-C₁₀)-cycloalkenylthio, (C₁-C₁₀)-alkylsulfinyl, (C₃-C₁₀)-alkenylsulfinyl, (C₃-C₁₀)-alkynylsulfinyl, (C₃-C₁₀)-cycloalkylsulfinyl, (C₄-C₁₀)-cycloalkenylsulfinyl, (C₃-C₁₀)-cycloalkyl-(C₁-C₄)-alkylsulfinyl, (C₄-C₁₀)-cycloalkenyl-(C₁-C₄)-alkylsulfinyl, (C₃-C₁₀)-cycloalkyl-(C₃-C₄)-alkenylsulfinyl, (C₄-C₁₀)-cycloalkenyl-(C₃-C₄)-alkenylsulfinyl, (C₁-C₄)-alkyl-(C₃-C₁₀)-cycloalkylsulfinyl, (C₂-C₄)-alkenyl-(C₃-C₁₀)-cycloalkylsulfinyl, (C₂-C₄)-alkynyl-(C₃-C₁₀)-cycloalkylsulfinyl, (C₁-C₄)-alkyl-(C₄-C₁₀)-cycloalkenylsulfinyl, (C₂-C₄)-alkenyl-(C₄-C₁₀)-cycloalkenylsulfinyl, (C₂-C₄)-alkynyl-(C₄-C₁₀)-cycloalkenylsulfinyl, (C₁-C₁₀)-alkylsulfonyl, (C₃-C₁₀)-alkenylsulfonyl, (C₃-C₁₀)-alkynylsulfonyl, (C₃-C₁₀)-cycloalkylsulfonyl, (C₄-C₁₀)-cycloalkenylsulfonyl, (C₃-C₁₀)-cycloalkyl-(C₁-C₄)-alkylsulfonyl, (C₄-C₁₀)-cycloalkenyl-(C₁-C₄)-alkylsulfonyl, (C₃-C₁₀)-cycloalkyl-(C₃-C₄)-alkenylsulfonyl, (C₄-C₁₀)-cycloalkenyl-(C₃-C₄)-alkenylsulfonyl, (C₁-C₄)-alkyl-(C₃-C₁₀)-cycloalkylsulfonyl, (C₂-C₄)-alkenyl-(C₃-C₁₀)-cycloalkylsulfonyl, (C₃-C₄)-alkynyl-(C₃-C₁₀)-cycloalkylsulfonyl, (C₁-C₄)-alkyl-(C₄-C₁₀)-cycloalkenylsulfonyl, (C₃-C₄)-alkenyl-(C₄-C₁₀)-cycloalkenylsulfonyl, (C₁-C₁₀)-alkylamino, (C₃-C₁₀)-alkenylamino, (C₃-C₁₀)-alkynylamino, (C₃-C₁₀)-cycloalkylamino, (C₄-C₁₀)-cycloalkenylamino, (C₃-C₁₀)-cycloalkyl-(C₁-C₄)-alkylamino, (C₄-C₁₀)-cycloalkenyl-(C₁-C₄)-alkylamino, (C₃-C₁₀)-cycloalkyl-(C₃-C₄)-alkenylamino, (C₄-C₁₀)-cycloalkenyl-(C₃-C₄)-alkenylamino, (C₁-C₄)-alkyl-(C₃-C₁₀)-cycloalkylamino, (C₂-C₄)-alkenyl-(C₃-C₁₀)-cycloalkylamino, (C₂-C₄)-alkynyl-(C₃-C₁₀)-cycloalkylamino, (C₁-C₄)-alkyl-(C₄-C₁₀)-cycloalkenylamino, (C₂-C₄)-alkenyl-(C₄-C₁₀)-cycloalkenylamino, the N-(C₁-C₄)-alkylamino analogs of the fourteen last-mentioned radicals, (C₁-C₁₀)-trialkylsilyl, aryl, aroyl, heterocyclylcarbonyl, aryloxy, arylthio, arylamino, N-(C₁-C₄)-alkyl-N-arylamino, aryl-(C₁-C₄)-alkoxy, aryl-(C₃-C₄)-alkenyloxy, aryl-(C₁-C₄)-alkylthio, aryl-(C₃-C₄)-alkenylthio, aryl-(C₁-C₄)-alkylamino, N-(C₁-C₄)-alkyl-N-aryl-(C₁-C₄)-alkylamino, aryl-(C₃-C₄)-alkenylamino, N-(C₁-C₄)-alkyl-N-aryl-(C₃-C₄)-alkenylamino, arylcarbamoyl, N-aryl-N-(C₁-C₄)-alkylcarbamoyl, aryl-(C₁-C₈)-dialkylsilyl, diaryl-(C₁-C₈)-alkylsilyl, triarylsilyl and 5- or 6-membered heterocyclyl, where the cyclic moiety of the 20 last-mentioned radicals is unsubstituted or substituted by one or more radicals selected from the group consisting of halogen, cyano, nitro, amino, hydroxyl, thio, (C₁-C₄)-alkyl, (C₁-C₄)-haloalkyl, (C₃-C₈)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, halo-(C₁-C₄)-alkylsulfonyl, (C₁-C₄)-alkylamino, trimethylsilyl and (C₁-C₄)-alkanoyl; where in the substituents mentioned above which can be present on the radicals R⁶ to R²⁶, hydrogen atoms attached to carbon are optionally replaced by up to three halogen atoms, in the case of fluorine also up to the maximum number.

6. An amide of the formula (Ia)-(Io) or a salt thereof 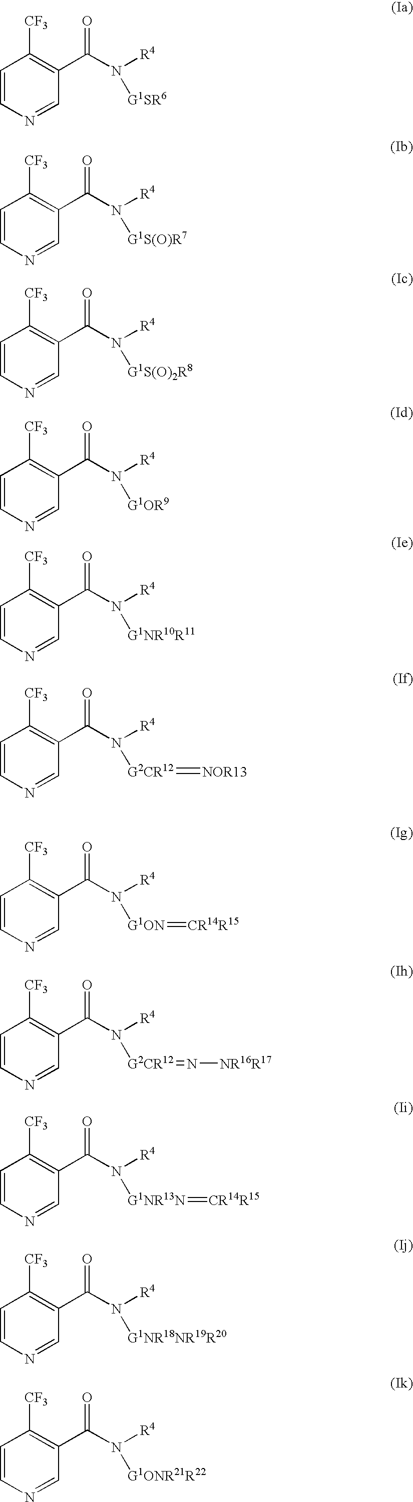 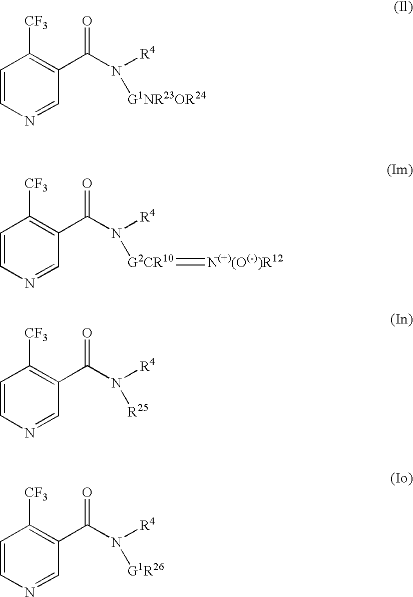
where the symbols and indices are as defined above in claim 1.

7. A process for preparing amides of the formula (I) as claimed in claim 1, where R¹, R², R³, R⁴, R⁵, A and n are as defined in claim 1 and Y is oxygen, comprising reacting a carboxylic acid of the formula (II) 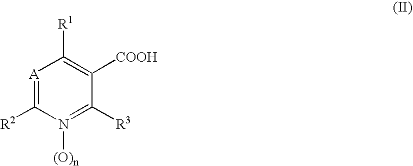
in which A, R¹, R², R³ and n are as defined in claim 1 in the form of an activated derivative of said acid in the presence of a base with a compound of the formula (III)
HNR⁴R⁵ (III)
in which R⁴ and R⁵ are as defined in claim 1;
followed, if desired, by further derivatizing the radicals R⁴ and R⁵.

8. A pesticidal composition comprising a Pesticidally effective amount of at least one compound as claimed in claim 1, and pesticidally acceptable additives or auxiliaries.

9. The pesticidal composition as claimed in claim 8, comprising a pesticidally effective amount of said at least one compound and an effective amount of at least one further active compound, which is selected from the group consisting of insecticides, attractants, sterilants, acaricides, nematicides, fungicides, growth regulants and herbicides and pesticidally acceptable auxiliaries or additives.

10. A pesticidal composition for use in the preservation of wood or as a preservative in sealants, in paints, in cooling lubricants for metal working or in drilling or cutting oils, comprising a pesticidally effective amount of at least one compound as claimed in claim 1, and auxiliaries or additives acceptable for use in those materials.

11. A veterinary medicament comprising a pesticidally effective amount of at least one compound as claimed in claim 1, and veterinarily acceptable auxiliaries or additives.

12. A method for protecting woods preservative, as a preservative in sealants, paints, cooling lubricants for metal working and/or drilling or cutting oils from harmful insects, arachnids, molluscs and/or nematodes, which comprises treating the wood, sealants, paints, cooling lubricants for metal working and/or drilling or cutting oils with a pesticidally effective amount of at least one compound as claimed in claim 1, or with a pesticidal composition comprising a pesticidally effective amount of said at least one compound and auxiliaries or additives acceptable for use in those materials.

13. A method for controlling harmful insects, arachnids, molluscs and/or nematodes in or on an animal which comprises administering to said animal a pesticidally effective amount of at least one compound as claimed in claim 1, or a veterinary medicament comprising a pesticidally effective amount of said at least one compound and veterinarily acceptable auxiliaries or additives.

14. A method for controlling harmful insects, arachnids, molluscs and/or nematodes, which comprises bringing said insects, arachnids, molluscs and/or nematodes into contact with a pesticidally effective amount of one at least one compound as claimed in claim 1 or of a pesticidal composition comprising a pesticidally effective amount of said at least one compound and pesticidally acceptable auxiliaries or additives.

15. A method for controlling harmful insects, arachnids, molluscs and/or nematodes, which comprises applying a pesticidally effective amount of at least one compound claimed in claim 1 or of a pesticidal composition comprising a pesticidally effective amount of said at least one compound and pesticidally acceptable auxiliaries or additives, onto said insects, arachnids, molluscs or nematodes or onto the plants, areas or substrates infested by them.

16. Seed, coated with or comprising a pesticidally effective amount of a compound as claimed in claim 1.

17. An amide of the formula (I) as claimed in claim 2, where R¹ is CF₃.

18. An amide of the formula (I) as claimed in claim 2, where
G¹ is CH₂—CH₂, CH₂—CH₂—CH₂, CH(CH₃)—CH₂ or CH(C₂H₅)—CH₂ and
G² is one of the groups defined above for G¹, CH₂ or CH(CH₃).

19. An amide of the formula (I) as claimed in claim 18, where R¹ is CF₃.

20. An amide of the formula (Ia)-(Io) as claimed in claim 6, where
R⁴ is hydrogen or methyl,
G¹ is CH₂—CH₂, CH₂—CH₂—CH₂, CH(CH₃)—CH₂ or CH(C₂H₅)—CH₂ and
G² is one of the groups defined above for G¹, CH₂ or CH(CH₃).
